SEQUENCE LISTING (22 sequences)

SEQ ID NO: 1 - protein, 44 aa, Artificial Sequence
  Peptide Antigen Conjugate Composition 1
  MISC_FEATURE (43)..(43): Xaa is Citrulline
  MISC_FEATURE (44)..(44): Xaa is Lys(N3-DBCO)-2B3W2
KKKKKKKKKVRGIPVHLELASMTNMELMSSIVHQQVFPTSPVXX

SEQ ID NO: 2 - protein, 41 aa, Artificial Sequence
  Peptide Antigen Conjugate Composition 2
  MISC_FEATURE (40)..(40): Xaa is Citrulline
  MISC_FEATURE (41)..(41): Xaa is Lys(N3-DBCO)-2B3W2
KKKKKKVRGRVLELFRAAQLANDVVLQIMELCGATRSPVXX

SEQ ID NO: 3 - protein, 40 aa, Artificial Sequence
  Peptide Antigen Conjugate Composition 3
  MISC_FEATURE (39)..(39): Xaa is Citrulline
  MISC_FEATURE (40)..(40): Xaa is Lys(N3-DBCO)-2B3W2
KKKKKKKVRKARDETAALLNSAVLGAAPLFVPPADSPVXX

SEQ ID NO: 4 - protein, 42 aa, Artificial Sequence
  Peptide Antigen Conjugate Composition 4
  MISC_FEATURE (41)..(41): Xaa is Citrulline
  MISC_FEATURE (42)..(42): Xaa is Lys(N3-DBCO)-2B3W2
KKKKKKKKKVRDFTGSNGDPSSPYSLHYLSPTGVNEYSPVXX

SEQ ID NO: 5 - protein, 40 aa, Artificial Sequence
  Peptide Antigen Conjugate Composition 5
  MISC_FEATURE (39)..(39): Xaa is Citrulline
  MISC_FEATURE (40)..(40): Xaa is Lys(N3-DBCO)-2B3W2
KKKKKKKVRSKLLSFMAPIDHTTMSDDARTELFRSSPVXX

SEQ ID NO: 6 - protein, 40 aa, Artificial Sequence
  Peptide Antigen Conjugate Composition 6
  MISC_FEATURE (39)..(39): Xaa is Citrulline
  MISC_FEATURE (40)..(40): Xaa is Lys(N3-DBCO)-2B3W2
KKKKKKVRREGVELCPGNKYEMRRHGTTHSLVIHDSPVXX

SEQ ID NO: 7 - protein, 38 aa, Artificial Sequence
  Peptide Antigen Conjugate Composition 7
  MISC_FEATURE (37)..(37): Xaa is Citrulline
  MISC_FEATURE (38)..(38): Xaa is Lys(N3-DBCO)-2B3W2
KKKKVRELINFKRKRVAAFQKNLIEMSELEIKHSPVXX

SEQ ID NO: 8 - protein, 39 aa, Artificial Sequence
  Peptide Antigen Conjugate Composition 8
  MISC_FEATURE (38)..(38): Xaa is Citrulline
  MISC_FEATURE (39)..(39): Xaa is Lys(N3-DBCO)-2B3W2
KKKKKVRDSGSPFPAAVILRDALHMARGLKYLHQSPVXX

SEQ ID NO: 9 - protein, 42 aa, Artificial Sequence
  Peptide Antigen Conjugate Composition 9
  MISC_FEATURE (41)..(41): Xaa is Citrulline
  MISC_FEATURE (42)..(42): Xaa is Lys(N3-DBCO)-2B3W2
KKKKKKKKVRSSPDEVALVEGVQSLGFTYLRLKDNYMSPVXX

SEQ ID NO: 10 - protein, 40 aa, Artificial Sequence
  Peptide Antigen Conjugate Composition 10
  MISC_FEATURE (39)..(39): Xaa is Citrulline
  MISC_FEATURE (40)..(40): Xaa is Lys(N3-DBCO)-2B3W2
KKKKKKVRFVVKAYLPVNESFAFTADLRSNTGGQASPVXX

SEQ ID NO: 11 - protein, 9 aa, Artificial Sequence
  MC-38 peptides (Adpgk)
ASMTNMELM

SEQ ID NO: 12 - protein, 9 aa, Artificial Sequence
  MC-38 peptides (Reps1)
AQLANDVVL

SEQ ID NO: 13 - protein, 9 aa, Artificial Sequence
  MC-38 peptides (Irgq)
AALLNSAVL

SEQ ID NO: 14 - protein, 9 aa, Artificial Sequence
  MC-38 peptides (Cpne1)
SSPYSLHYL

SEQ ID NO: 15 - protein, 9 aa, Artificial Sequence
  MC-38 peptides (Aaltf)
MAPIDHTTM

SEQ ID NO: 16 - protein, 9 aa, Artificial Sequence
  B16.F10 peptides (Obsl1)
LCPGNKYEM

SEQ ID NO: 17 - protein, 10 aa, Artificial Sequence
  B16.F10 peptides (Snx5 (R373Q))
AAFQKNLIEM

SEQ ID NO: 18 - protein, 9 aa, Artificial Sequence
  B16.F10 peptides (Pbk)
AAVILRDAL

SEQ ID NO: 19 - protein, 8 aa, Artificial Sequence
  B16.F10 peptides (Atp11a)
QSLGFTYL

SEQ ID NO: 20 - protein, 15 aa, Artificial Sequence
  B16.F10 peptides (Eef2)
VKAYLPVNESFAFTA

SEQ ID NO: 21 - protein, 8 aa, Artificial Sequence
  Maraba N52-59 peptide
RGYVYQGL

SEQ ID NO: 22 - protein, 9 aa, Artificial Sequence
  FMT N301-309
AVVLMFAQC

(19) United States
(12) Patent Application Publication
(10) Pub. No.: US 2022/0305099 A1
(43) Pub. Date: Sep. 29, 2022

(54) METHODS FOR INDUCING AN IMMUNE RESPONSE AGAINST NEOANTIGENS

(71) Applicant: TURNSTONE BIOLOGICS CORP., New York, NY (US)

(72) Inventors: David STOJDL, Ottawa (CA); Geoffrey Martin LYNN, Baltimore, MD (US); Andrew Scott ISHIZUKA, Washington, DC (US)

(73) Assignee: TURNSTONE BIOLOGICS CORP., New York, NY (US)

(21) Appl. No.: 17/638,576

(22) PCT Filed: Aug. 26, 2020
(86) PCT No.: PCT/US2020/047962

§ 371 Date: Feb. 25, 2022

Related U.S. Application Data
(60) Provisional application No. 62/892,534, filed on Aug. 27, 2019.

(51) Int. Cl. A61K 39/00; A61K 35/766; A61K 35/768

(52) U.S. Cl. CPC A61K 39/0011; A61K 35/766; A61K 35/768; A61K 2039/5256; A61K 2039/55516; A61K 2039/60

(57) ABSTRACT

Provided herein is a method for inducing an immune response to at least one neoantigen, the method comprising administering to a subject a priming composition comprising a peptide antigen conjugate and at least a first boost. The first boost comprises a first oncolytic virus comprising a genome that expresses a first peptide or a second peptide, wherein the first and second peptide are each capable of inducing an immune response to at least one neoantigen. The method further comprises administering the subject a second boost, comprising a second oncolytic virus comprising a genome that expresses a third peptide or a fourth peptide, wherein the third peptide and the fourth peptide are each capable of inducing an immune response to at least one neoantigen, and wherein the second oncolytic virus is immunologically distinct from the first oncolytic virus. The subject may have pre-existing immunity to the at least one neoantigen.

### CROSS-REFERENCE TO RELATED APPLICATIONS

This application claims the benefit of U.S. Provisional Application No. 62/892,534, filed on Aug. 27, 2019, which is incorporated by reference herein in its entirety.

### REFERENCE TO SEQUENCE LISTING SUBMITTED ELECTRONICALLY

This application incorporates by reference a Sequence Listing submitted with this application as a text file in ASCII format entitled “14596-005-228_SEQ_LISTING.txt” created on Aug. 25, 2020 and having a size of 9,091 bytes.

### 1. INTRODUCTION

In one aspect, provided herein is a method for inducing an immune response to at least one neoantigen, the method comprising administering to a subject a priming composition comprising a peptide antigen conjugate and at least a first boost, wherein the first boost comprises a first oncolytic virus comprising a genome that expresses, in the subject, a first peptide, or the first boost comprises a first oncolytic virus and a second peptide, wherein the first peptide and the second peptide are each capable of inducing an immune response to at least one neoantigen. In a specific aspect, the method further comprises administering the subject a second boost, wherein the second boost comprises a second oncolytic virus comprising a genome that expresses, in the subject, a third peptide, or the second boost comprises a second oncolytic virus and a fourth peptide, wherein the third peptide and the fourth peptide are each capable of inducing an immune response to at least one neoantigen, and wherein the second oncolytic virus is immunologically distinct from the first oncolytic virus. The subject may have pre-existing immunity to the at least one neoantigen.

### 2. BACKGROUND

An oncolytic prime:boost strategy based on a single tumour antigen target can achieve robust protection in the prophylactic setting. Yet in the therapeutic setting, tumour-bearing animal model systems demonstrate rapid tumour regression following oncolytic immunotherapy but often fail to achieve long-term cures, with tumours recurring following treatment. Several additional in vivo studies have shown similar outcomes following immunotherapeutic approaches based on a single antigen target. This effect can be the result of antigen loss in response to therapeutic pressure (Rommelfanger et al., Cancer Res. 2012; 72(18):4753-4764; Khong et al., J Immunother. 2004; 27(3):184-190; Mackensen et al., J Clin Oncol. 2006; 24(31):5060-5069; Yee C et al., Proc Natl Acad Sci USA. 2002; 99(25):16168-16173). However, antigen-targeted T cell therapies can still fail to generate durable cures in 80-90% of animals even when tumours continue to robustly express the targeted antigen, and relapsed tumours can regain responsiveness to antigen-targeted therapies following tumour re-transplantation into naive animals (Straetemans et al., Mol Ther. 2015; 23(2):396-406), suggesting a role for immunosuppressive mechanisms in addition to bona fide antigen loss. Since immunotherapies targeted towards more than one tumour antigen typically achieve longer-term control (Rommelfanger et al., Cancer Res. 2012; 72(18):4753-4764; Anurathapan et al., Mol Ther. 2014; 22(3):623-633; Hegde et al., Mol Ther. 2013; 21(11):2087-2101), there is clear therapeutic value in exploring large-scale tumour antigen library targets.

Neoepitopes are peptide epitopes that arise from the genetic aberrations within the tumour. These mutations convert self-epitopes that would otherwise be tolerated by T cells in the periphery into immunogenic foreign epitopes capable of engaging circulating T cells. Importantly, this means that neoantigen-specific CD8+ T cells often show exquisite specificity for mutant (non-self) over wild-type (self) proteins (Nielsen et al., Clin Cancer Res. 2016; 22(9):2226-2236).

Tumours are genetically complex tissues that present with extreme levels of inter- and intra-patient heterogeneity. Multiple clones ranging from 2 to >20 (depending on the cancer indication) can be identified within a single tumour (Andor et al., Nat Med. 2016; 22(1):105-113; Ling et al., Proc Natl Acad Sci USA. 2015; 112(47):E6496-6505). Multi-sample whole exome sequencing analysis demonstrates that a single tumour mass has an extremely high genetic diversity, with more than 1,000,000 mutations in coding regions (Ling et al., Proc Natl Acad Sci USA. 2015; 112(47):E6496-6505). Between 8-78% of neoantigens are located in specific subclonal populations (McGranahan N, Furness A J, Rosenthal R, et al. Clonal neoantigens elicit T cell immunoreactivity and sensitivity to immune checkpoint blockade. Science. 2016; 351(6280): 1463-1469).

In human patients, increased neoantigen load is associated with elevated frequencies of CD8+ T cells at the tumour site (Brown et al., Genome research. 2014; 24(5):743-750), and tumour neoantigen burden correlates with overall survival following checkpoint blockade (McGranahan N, Furness A J, Rosenthal R, et al. Clonal neoantigens elicit T cell immunoreactivity and sensitivity to immune checkpoint blockade. Science. 2016; 351(6280):1463-1469; Brown et al., Genome research. 2014; 24(5):743-750; Strickland et al., Oncotarget. 2016; 7(12):13587-13598; Rizvi et al., Science. 2015; 348(6230):124-128; Giannakis et al., Genomic Correlates of Immune-Cell Infiltrates in Colorectal Carcinoma. Cell Rep. 2016; 15(4):857-865). Thus, there is clear therapeutic value in targeting neoantigens in the oncolytic vaccine setting.

### 3. SUMMARY

In one aspect, provided herein is a method of inducing an immune response to at least one neoantigen in a subject, the method comprising: (a) administering to the subject a dose of a priming composition that is capable of inducing an immune response to the at least one neoantigen, such as described in Section 5.3 of 6, infra; and (b) subsequently administering to the subject a first boost, such as described in Section 5.4 or 6, infra. In certain embodiments, the method further comprises: (c) subsequently administering to the subject a second boost, such as described in Section 5.4 or 6, infra. See, e.g., Sections 5.5 and 6 for methods for inducing an immune response to at least one neoantigen in a subject. In certain embodiments, the priming composition comprises a protein that comprises an antigenic protein, wherein the protein is linked to a hydrophobic molecule or a particle, directly or indirectly. In a specific embodiment, the protein is linked to the hydrophobic molecule or particle via an N-terminal or C-terminal extension. In some embodiments, the protein is linked to the hydrophobic molecule or particle via a linker. In certain embodiments, the protein that comprises an antigenic protein further comprises an amino acid sequence that enhance the solubility of the protein. In some embodiments, the protein that comprises an antigenic protein further comprises an amino acid sequence that enhances intracellular release of the protein. In certain embodiments, the priming composition comprises an adjuvant. In a specific embodiment, the adjuvant is a TLR 7/8 adjuvant. In some embodiments, the adjuvant is linked to the protein in the priming composition. In certain embodiments, the priming composition self-assembles into nanoparticle micelles in aqueous solution. In a specific embodiment, the priming composition comprises an antigen peptide conjugate described in Section 5.2.

The present disclosure is based, in part, on the finding that use of at least two immunologically distinct oncolytic viruses significantly increase neoantigen-specific CD8+ T cell-mediated immune responses when administered in a sequential heterologous boost (“superboost”) treatment regimen. See, e.g., Section 5.4 and 5.5, infra, for boosts and methods for inducing an immune response to a neoantigen using a sequential heterologous boost.

In another aspect, provided herein is a method of inducing an immune response to at least one neoantigen in a subject, the method comprising: (a) administering to the subject a dose of a priming composition that is capable of inducing an immune response to the at least one neoantigen, the priming composition comprising a peptide antigen conjugate that comprises (1) an antigenic protein (A) and (2) either a hydrophobic molecule (H) or a particle (P), wherein the antigenic protein (A) is linked to either the hydrophobic molecule (H) or the particle (P) directly or indirectly via an optional N-terminal extension (B1) that is linked to the N-terminus of the antigenic protein (A) or an optional C-terminal extension (B2) that is linked to the C-terminus of the antigenic protein (A), optionally wherein the hydrophobic molecule (H) or Particle (P) is linked to the extension (B1 or B2) indirectly via a Linker (L); and (b) subsequently administering to the subject a first boost comprising a dose of a first composition, wherein the first composition comprises a first oncolytic virus comprising a genome that comprises a first transgene, wherein the first transgene encodes and expresses a first protein in the subject, and wherein the first protein or a fragment thereof is capable of inducing an immune response to the at least one neoantigen. In certain embodiments, the method further comprises: (c) subsequently administering to the subject a second boost comprising (i) a dose of a second composition, wherein the second composition comprises a second oncolytic virus and a first peptide composition, or (ii) a dose of a third composition and a dose of a fourth composition, wherein the third composition comprises the second oncolytic virus, and the fourth composition comprises the first peptide composition, wherein the first peptide composition is capable of inducing an immune response to the at least one neoantigen, wherein the second oncolytic virus is immunologically distinct from the first oncolytic virus, and wherein the third and fourth compositions are administered concurrently or sequentially to the subject. In some embodiments, the method further comprises: (c) subsequently administering to the subject a second boost comprising a dose of a second composition, wherein the second composition comprises a second oncolytic virus that comprises a genome comprising a second transgene, wherein the second transgene encodes and expresses a second protein in the subject, wherein the second protein or a fragment thereof is capable of inducing an immune response to the at least one neoantigen, and wherein the second oncolytic virus is immunologically distinct from the first oncolytic virus.

In another aspect, provided herein is a method of inducing an immune response to at least one neoantigen in a subject, the method comprising: (a) administering to the subject a dose of a priming composition that is capable of inducing an immune response to the at least one neoantigen, the priming composition comprising a peptide antigen conjugate that comprises (1) an antigenic protein (A) and (2) either a hydrophobic molecule (H) or a particle (P), wherein the antigenic protein (A) is linked to either the hydrophobic molecule (H) or the particle (P) directly or indirectly via an optional N-terminal extension (B1) that is linked to the N-terminus of the antigenic protein (A) or an optional C-terminal extension (B2) that is linked to the C-terminus of the antigenic protein (A), optionally wherein the hydrophobic molecule (H) or Particle (P) is linked to the extension (B1 or B2) indirectly via a Linker (L); and (b) administering to the subject a first boost comprising (i) a dose of a first composition comprising a first oncolytic virus and a first peptide composition, or (ii) a dose of a second composition and a dose of a third composition, wherein the second composition comprises the first oncolytic virus, and the third composition comprises the first peptide composition, wherein the first peptide composition is capable of inducing an immune response to the at least one neoantigen, and wherein the second and third compositions are administered concurrently or sequentially to the subject. In certain embodiments, the method further comprises: (c) subsequently administering to the subject a second boost comprising a dose of a fourth composition, wherein the fourth composition comprises a second oncolytic virus that comprises a genome comprising a first transgene, wherein the first transgene encodes and expresses a first protein in the subject, wherein the first protein or a fragment thereof is capable of inducing an immune response to the at least one neoantigen, and wherein the second oncolytic virus is immunologically distinct from the first oncolytic virus. In some embodiments, the method further comprises: (c) subsequently administering to the subject a second boost comprising (i) a dose of a fourth composition, wherein the fourth composition comprises a second oncolytic virus and a second peptide composition, or (ii) a dose of a fifth composition and a dose of a sixth composition, wherein the fifth composition comprises the second oncolytic virus, and the sixth composition comprises the second peptide composition, wherein the second peptide composition is capable of inducing an immune response to the at least one neoantigen, wherein the second oncolytic virus is immunologically distinct from the first oncolytic virus, and wherein the fifth and sixth compositions are administered concurrently or sequentially to the subject.

In another aspect, provided herein is a method of inducing an immune response to at least one neoantigen in a subject, the method comprising administering to the subject a first boost comprising a dose of a first composition, wherein the first composition comprises a first oncolytic virus comprising a genome that comprises a first transgene, wherein the first transgene encodes and expresses a first protein in the subject, and wherein the first protein or a fragment thereof is capable of inducing an immune response to the at least one neoantigen, wherein the subject was previously administered a dose of a priming composition that is capable of inducing an immune response to the least one neoantigen, the priming composition comprising a peptide antigen conjugate that comprises (1) an antigenic protein (A) and (2) either a hydrophobic molecule (H) or a particle (P), wherein the antigenic protein (A) is linked to either the hydrophobic molecule (H) or the particle (P) directly or indirectly via an optional N-terminal extension (B1) that is linked to the N-terminus of the antigenic protein (A) or an optional C-terminal extension (B2) that is linked to the C-terminus of the antigenic protein (A), optionally wherein the hydrophobic molecule (H) or Particle (P) is linked to the extension (B1 or B2) indirectly via a Linker (L). In certain embodiments, the method further comprises: (c) subsequently administering to the subject a second boost comprising (i) a dose of a second composition, wherein the second composition comprises a second oncolytic virus and a first peptide composition, or (ii) a dose of a third composition and a dose of a fourth composition, wherein the third composition comprises the second oncolytic virus, and the fourth composition comprises the first peptide composition, wherein the first peptide composition is capable of inducing an immune response to the at least one neoantigen, wherein the second oncolytic virus is immunologically distinct from the first oncolytic virus, and wherein the third and fourth compositions are administered concurrently or sequentially to the subject. In some embodiments, the method further comprises: (c) subsequently administering to the subject a second boost comprising a dose of a second composition, wherein the second composition comprises a second oncolytic virus that comprises a genome comprising a second transgene, wherein the second transgene encodes and expresses a second protein in the subject, wherein the second protein or a fragment thereof is capable of inducing an immune response to the at least one neoantigen, and wherein the second oncolytic virus is immunologically distinct from the first oncolytic virus.

In another aspect, provided herein is a method of inducing an immune response to at least one neoantigen in a subject, the method comprising administering to the subject a first boost comprising (i) a dose of a first composition comprising a first oncolytic virus and a first peptide composition, or (ii) a dose of a second composition and a dose of a third composition, wherein the second composition comprises the first oncolytic virus, and the third composition comprises the first peptide composition, wherein the first peptide composition is capable of inducing an immune response to the at least one neoantigen, and wherein the second and third compositions are administered concurrently or sequentially to the subject, wherein the subject was previously administered a dose of a priming composition that is capable of inducing an immune response to the least one neoantigen, the priming composition comprising (1) an antigenic protein (A) and (2) either a hydrophobic molecule (H) or a particle (P), wherein the antigenic protein (A) is linked to either the hydrophobic molecule (H) or the particle (P) directly or indirectly via an optional N-terminal extension (B1) that is linked to the N-terminus of the antigenic protein (A) or an optional C-terminal extension (B2) that is linked to the C-terminus of the antigenic protein (A), optionally wherein the hydrophobic molecule (H) or Particle (P) is linked to the extension (B1 or B2) indirectly via a Linker (L). In certain embodiments, the method further comprises: (c) subsequently administering to the subject a second boost comprising a dose of a fourth composition, wherein the fourth composition comprises a second oncolytic virus that comprises a genome comprising a first transgene, wherein the first transgene encodes and expresses a first protein in the subject, wherein the first protein or a fragment thereof is capable of inducing an immune response to the at least one neoantigen, and wherein the second oncolytic virus is immunologically distinct from the first oncolytic virus. In some embodiments, the method further comprises: (c) subsequently administering to the subject a second boost comprising (i) a dose of a fourth composition, wherein the fourth composition comprises a second oncolytic virus and a second peptide composition, or (ii) a dose of a fifth composition and a dose of a sixth composition, wherein the fifth composition comprises the second oncolytic virus, and the sixth composition comprises the second peptide composition, wherein the second peptide composition is capable of inducing an immune response to the at least one neoantigen, wherein the second oncolytic virus is immunologically distinct from the first oncolytic virus, and wherein the fifth and sixth compositions are administered concurrently or sequentially to the subject.

The proteins and peptide compositions used in the methods described herein may comprise amino acid sequences that are the same or different. In some embodiments, the proteins and peptide compositions comprise amino acid sequences that overlap. In other embodiments, the proteins and peptide compositions comprise amino acid sequences that are identical. In certain embodiments, the proteins and peptide compositions each comprise at least one epitope of a neoantigen in common.

In certain embodiments, the amino acid sequence of the first protein is identical to the amino acid sequence of the second protein. In some embodiments, the first protein and the first peptide composition comprise identical amino acid sequences. In certain embodiments, the first protein and the first peptide composition comprise amino acid sequences that contain the same or overlapping epitopes.

In certain embodiments, the amino acid sequence of the second protein is different than the amino acid sequence of first protein or the first peptide composition. In other embodiments, the amino acid sequence of the second protein is identical to the amino acid sequence of first protein or the first peptide composition. In some embodiments, the amino acid sequence of the second protein includes at least one epitope found in the first protein.

In certain embodiments, the amino acid sequence of the second peptide composition is different from the amino acid sequence of the first protein or the first peptide composition. In other embodiments, the amino acid sequence of the second peptide composition is identical to the amino acid sequence of the first protein or the first peptide composition. In some embodiments, the amino acid sequence of the second peptide composition includes at least one epitope found in the first peptide composition.

In certain embodiments, a dose of a priming composition is administered 7 to 21 days before the first boost. In other embodiments, a dose of a priming composition is administered 2 weeks to 3 months before the first boost.

In certain embodiments, the second boost is administered 7 to 21 days after the first boost. In other embodiments, the second boost is administered 2 weeks to 3 months after the first boost.

In certain embodiments, the first oncolytic virus, the second oncolytic virus or both are attenuated. In a specific embodiment, the first oncolytic virus, the second oncolytic viruses, or both are rhabdoviruses. In another embodiment, the first oncolytic virus or the second oncolytic virus is a vaccinia virus, an adenovirus, a measles virus, or a vesicular stomatitis virus. In a specific embodiment, the vaccinia virus is Copenhagen, Western Reserve, Wyeth, Tian Tan or Lister. In another embodiment, the first or second oncolytic virus is a Maraba virus. In a specific embodiment, the Maraba virus is MG1.

In another embodiment, the first or second oncolytic virus is a Farmington (FMT) virus. In another embodiment, the first oncolytic virus is a Maraba virus and the second oncolytic virus is a Farmington virus. In another embodiment, the first oncolytic virus is a Farmington virus and the second oncolytic virus is a Maraba virus. In another embodiment, the first oncolytic virus is a vaccinia virus and the second oncolytic virus is a Maraba virus. In another embodiment, the first oncolytic virus is a Maraba virus and the second oncolytic virus is a vaccinia virus. In another embodiment, the first oncolytic virus is a vaccinia virus and the second oncolytic virus is a Farmington virus. In another embodiment, the first oncolytic virus is a Farmington virus and the second oncolytic virus is a vaccinia virus.

In a specific embodiment, a boost of an oncolytic virus comprises 10⁷ to 10¹² PFU. In another embodiment, a first boost of an oncolytic virus comprises 10⁷ to 10¹² PFU, and a second boost of an oncolytic virus comprises 10⁷ to 10¹² PFU.

In a specific embodiment, a priming composition is administered to a subject in accordance with the methods described herein intravenously, subcutaneously or intramuscularly. In another embodiment, a boost is administered to a subject in accordance with the methods described herein intravenously, subcutaneously or intramuscularly. In another embodiment, a priming composition and a boosting composition are administered to a subject in accordance with the methods described herein intravenously, subcutaneously or intramuscularly. In another embodiment, the first and second boosts are administered to a subject in accordance with the methods described herein intravenously, subcutaneously or intramuscularly. In another embodiment, a priming composition and the first and second boots are administered to a subject in accordance with the methods described herein intravenously, subcutaneously or intramuscularly.

In certain embodiments, a subject is determined to have pre-existing immunity to a neoantigen. In a specific embodiment, the subject is determined to have pre-existing immunity by measuring the number of antigen-specific interferon gamma-positive CD8+ T cells per ml of peripheral blood from the subject. In a specific embodiment, a subject is a mammal. In another specific embodiment, a subject is a human.

### 4. BRIEF DESCRIPTION OF THE DRAWINGS

FIG. 1. Experimental protocol for the results in FIGS. 2-9. This figure shows that a prime (PBS, Adjuvant+MC38 loose peptides administered subcutaneously (SC), Adjuvant+B16 loose peptides administered subcutaneously (SC), AVT01 MC38 M05 (4 nmol) or AVT01 B16 M05 (4 nmol) administered intramuscularly (IM)) was administered to mice at day 0 and a boost with PBS or 3×10⁸ PFU of MG1-N10 was administered intravenously (IV) to mice at day 14. The peptide concentration in prime formulations is 50 μg per mouse. The adjuvant (Adj.) in prime formulations is composed of 10 μg/mouse of poly I:C and 30 μg/mouse anti-CD40 antibody. The MG1-N10 is MG1 virus engineered to express a total of ten neoantigens (a combination of five MC38 and five B16 neoantigens) as listed in Table 1. Blood samples were taken on days 13 and 20.

TABLE 1
10 Neoantigens Identified in Specific Cancer Cell Models

| | |
|---|---|
| MC38 | Adpgk |
| MC38 | Reps1 |
| MC38 | Iraq |
| MC38 | Cpne1 |
| MC38 | Aatf |
| B16-M27 | Obsl1 |
| B16-M39 | Snx5 (R373Q) |
| B16-M33 | Pbk |
| B16-M21 | Atp11a |
| B16-M05 | Eef2 |

FIG. 2. Cumulative immune response readout as intracellular IFN-γ expression in CD8+ Tcells. Blood was sampled 6 days post boost. This figure shows the pooled sum of numbers of CD8+ T cells in peripheral blood expressing IFNγ in response to ex-vivo stimulation with individual minimal epitopes corresponding to neoantigens (MC38) used for vaccination from (1) naïve control mice primed with PBS and boosted with PBS; (2) naïve mice primed with 50 μl of PBS administered intramuscularly (IM) and boosted with 3×10⁸ PFU of MG1-N10 intravenously(IV); (3) naïve mice primed with Adj+MC38 loose peptides (50 μg of each peptide administered subcutaneously (SC)) and boosted with 3×10⁸ PFU of MG1-N10 intravenously (IV); and (4) naïve mice primed with 4 nmol (2 nmol per injection site (IM)) of AVT01 M05 MC38 intramuscularly (IM) and boosted with 3×10⁸ PFU of MG1-N10 intravenously (IV).

FIG. 3. Individual immune response readout as intracellular IFNγ expression in CD8+ T cells. Blood was sampled 6 days post boost. This figure shows the individual numbers of CD8+ T cells in peripheral blood expressing IFNγ in response to ex-vivo stimulation with individual minimal epitopes corresponding to neoantigens (MC38) used for vaccination from (1) naïve control mice primed with PBS and boosted with PBS; (2) naïve mice primed with 50 μl of PBS intramuscularly (IM) and boosted with 3×10⁸ PFU of MG1-N10 intravenously (IV); (3) naïve mice primed with Adj+MC38 loose peptides (50 μg of each peptide administered subcutaneously (SC)) and boosted with 3×10⁸ PFU of MG1-N10 intravenously (IV); and (4) naïve mice primed with 4 nmol (2 nmol per injection site (IM)) of AVT01 M05 M38 intramuscularly (IM) and boosted with 3×10⁸ PFU of MG1-N10 intravenously (IV).

FIG. 4. Cumulative immune response readout as intracellular IFN-γ expression in CD8+ T cells. Blood was sampled 6 days post boost. This figure shows the pooled sum of numbers of CD8+ T cells in peripheral blood expressing IFNγ in response to ex-vivo stimulation with individual minimal epitopes corresponding to neoantigens (B16) used for vaccination from (1) naïve control mice primed with PBS and boosted with PBS; (2) naïve mice primed with 50 μl of PBS intramuscularly (IM) and boosted with 3×10⁸ PFU of MG1-N10 intravenously (IV); (3) naïve mice primed with Adj+B16 loose peptides (50 μg of each peptide administered subcutaneously (SC)) and boosted with 3×10⁸ PFU of MG1-N10 intravenously (IV); and (4) naïve mice primed with 4 nmol (2 nmol per injection site (IM)) of AVT01 M05 B16 intramuscularly (IM) and boosted with 3×10⁸ PFU of MG1-N10 intravenously (IV).

FIG. 5. Individual immune response readout as intracellular IFN-γ expression in CD8+ T cells. Blood was sampled 6 days post boost. This figure shows the individual numbers of CD8+ T cells in peripheral blood expressing IFNγ in response to ex-vivo stimulation with individual minimal epitopes corresponding to neoantigens (B16) used for vaccination (1) naïve control mice primed with PBS and boosted with PBS; (2) naïve mice primed with 50 μl of PBS intramuscularly (IM) and boosted with 3×10⁸ PFU of MG1-N10 intravenously (IV); (3) naïve mice primed with Adj+B16 loose peptides (50 μg of each peptide administered subcutaneously (SC)) and boosted with 3×10⁸ PFU of MG1-N10 intravenously (IV); and (4) naïve mice primed with 4 nmol (2 nmol per injection site (IM)) of AVT01 M05 B16 intramuscularly and boosted with 3×10⁸ PFU of MG1-N10 intravenously (IV).

FIG. 6. Cumulative immune response readout as intracellular IFN-γ expression in CD8+ T cells. Blood was sampled 30 days post-boost. This figure shows the pooled sum of numbers of CD8+ T cells in peripheral blood 30 days post boost expressing IFNγ in response to ex-vivo stimulation with individual minimal epitopes corresponding to neoantigens (MC38) from (1) naïve control mice primed with PBS and boosted with PBS; (2) naïve mice primed with 50 μl of PBS intramuscularly (IM) and boosted with 3×10⁸ PFU of MG1-N10 intravenously (IV); (3) naïve mice primed with Adj+MC38 loose peptides (50 μg of each peptide administered subcutaneously (SC)) and boosted with 3×10⁸ PFU of MG1-N10 intravenously (IV); and (4) naïve mice primed with 4 nmol (2 nmol per injection site (IM)) of AVT01 M05 MC38 intramuscularly (IM) and boosted with 3×10⁸ PFU of MG1-N10 intravenously (IV).

FIG. 7. Individual immune response readout as intracellular IFNγ expression with CD8+ T cells. Blood was sampled 30 days post-boost. This figure shows the individual numbers of CD8+ T cells in peripheral blood 30 days post boost expressing IFNγ in response to ex-vivo stimulation with individual minimal epitopes corresponding to neoantigens (MC38) from (1) naïve control mice primed with PBS and boosted with PBS; (2) naïve mice primed with 50 μl of PBS intramuscularly (IM) and boosted with 3×10⁸ PFU of MG1-N10 intravenously (IV); (3) naïve mice primed with Adj+MC38 loose peptides (50 μg of each peptide administered subcutaneously (SC)) and boosted with 3×10⁸ PFU of MG1-N10 intravenously (IV); and (4) naïve mice primed with 4 nmol (2 nmol per injection site (IM)) of AVT01 M05 MC38 intramuscularly (IM) and boosted with 3×10⁸ PFU of MG1-N10 intravenously (IV).

FIG. 8. Cumulative immune response readout as intracellular IFN-γ expression in CD8+ T cells. Blood was sampled 30 days post-boost. This figure shows the pooled sum of numbers of CD8+ T cells in peripheral blood 30 days post boost expressing IFNγ in response to ex-vivo stimulation with individual minimal epitopes corresponding to neoantigens (B16) from (1) naïve control mice primed with PBS and boosted with PBS; (2) naïve mice primed with 50 μl of PBS intramuscularly (IM) and boosted with 3×10⁸ PFU of MG1-N10 intravenously (IV); (3) naïve mice primed with Adj+B16 loose peptides (50 μg of each peptide administered subcutaneously (SC)) and boosted with 3×10⁸ PFU of MG1-N10 intravenously (IV); and (4) naïve mice primed with 4 nmol (2 nmol per injection site (IM)) of AVT01 M05 B16 intramuscularly (IM) and boosted with 3×10⁸ PFU of MG1-N10 intravenously (IV).

FIG. 9. Individual immune response readout as intracellular IFN-γ expression with CD8+ T cells. Blood was sampled 30 days post-boost. This figure shows the individual number of CD8+ T cells in peripheral blood 30 days post boost expressing IFNγ in response to ex-vivo stimulation with individual minimal epitopes corresponding to neoantigens (B16) from (1) naïve control mice primed with PBS and boosted with PBS; (2) naïve mice primed with 50 μl of PBS intramuscularly (IM) and boosted with 3×10⁸ PFU of MG1-N10 intravenously (IV); (3) naïve mice primed with Adj+B16 loose peptides (50 μg of each peptide administered subcutaneously (SC)) and boosted with 3×10⁸ PFU of MG1-N10 intravenously (IV); and (4) naïve mice primed with 4 nmol (2 nmol per injection site (IM)) of AVT01 M05 B16 intramuscularly and boosted with 3×10⁸ PFU of MG1-N10 intravenously (IV).

FIG. 10. Experimental protocol for the results in FIGS. 11-12. This figure shows that a prime (PBS or AVT01 M10 (2 nmol)) was administered intramuscularly (IM) to mice at day 0 and a boost with 3×10⁸ PFU of MG1-N10 or 3×10⁸ PFU MG1-nr plus 10 peptides representing minimal epitopes corresponding to neo-antigens encoded in MG1-N10 (50 μg per peptide) was administered intravenously (IV) to mice at day 14. The MG1-N10 is MG1 virus engineered to express a total of ten neoantigens (a combination of 5 MC38 and 5 B16 peptides as listed in Table 1).

FIG. 11. Cumulative immune response readout as intracellular IFNγ expression in CD8+ T cells following ex-vivo stimulation with individual minimal epitopes corresponding to neo-antigens (MC38 and B16) used for vaccination. Blood was sampled 6 days post boost. This figure shows the pooled sum of numbers of CD8+ T cells in peripheral blood expressing IFNγ in response to ex-vivo stimulations with individual minimal epitopes corresponding to neo-antigens (MC38 and B16) used for vaccination from mice primed with AVT01-M10 (2 nmol (1 nmol per injection site (IM)) and boosted with 3×10⁸ PFU of MG1nr intravenously (IV) plus N10 (50 μg per peptide per mouse); mice primed with AVT01-M10 (2 nmol (1 nmol per injection site (IM)) and boosted with 3×10⁸ PFU of MG1-N10 intravenously (IV); and a naïve control group (received PBS as prime and boost).

FIG. 12. Individual immune response readout as intracellular IFNγ expression within CD8+ T cells compartment. Blood was sampled 6 days post boost. This figure shows the individual numbers of CD8+ T cells in peripheral blood expressing IFNγ in response to ex-vivo stimulations with individual minimal epitopes corresponding to neo-antigens (MC38 and B16) used for vaccination from mice primed with AVT01-M10 (2 nmol (1 nmol per injection site (IM)) and boosted with 3×10⁸ PFU of MG1nr intravenously (IV) plus N10 (50 μg of each peptide per mouse); mice primed with AVT01-M10 (2 nmol (1 nmol per injection site (IM)) and boosted with 3×10⁸ PFU of MG1-N10 intravenously (IV); and a naïve control group.

FIG. 13. Experimental protocol for the results in FIG. 14. Each treatment group received a prime of 8 nmol of AVT01 individual neoantigen (one of the N10 antigens, each group received different antigens (AVT01 M10)) administered intramuscularly (IM) to mice at day 0. All mice received a first boost with np or 3×10⁸ PFU of MG1-N10 administered intravenously (IV) at day 14, and a second boost of 3×10⁸ PFU of FMT-N10 administered intravenously (IV) at day 67. The FMT-N10 and MG1-N10 viruses were engineered to express a total of ten neoantigens (a combination of MC38 and B16 peptides as listed in Table 1). Blood samples were taken at day 20 (6 days post boost 1) and day 74 (7 days post boost 2).

FIG. 14. Superboost with FMT-N10 increases the magnitude of immune response to MG1-N10 vaccinated mice after priming with AVT01 M10. This figure shows numbers of CD8+ T cells expressing IFNγ in response to ex vivo stimulation with minimal epitopes corresponding to neo-antigens (MC38 and B16) used for vaccination in peripheral blood after boost 1 (MG1-N10 bars labelled “1”) and after boost 2 (FMT-N10 bars labelled “2”).

FIG. 15. Peptide Antigen Conjugate Cartoon of formula C-B1-A-B2-L-H, wherein C is a charged molecule; B1 and B2 are N- and C-terminal extensions; A is an antigenic protein; L is a linker; and, H is a hydrophobic block (sometimes referred to as a “hydrophobic molecule”).

FIGS. 16A-16B. Cumulative immune response readout as intracellular IFN-γ expression in CD8+ T cells. FIG. 16A provides the experimental protocol for the results in FIG. 16B. FIG. 16B shows the pooled sum of numbers of CD8+ T cells in peripheral blood expressing IFNγ in response to ex-vivo stimulation with individual minimal epitopes corresponding to neoantigens (MC38) used for vaccination. Mice were primed on day 1 with 10 nmol AVT01 MC38 M05 either short (˜9mer) or long (˜25mer) peptides. Mice were boosted on day 15 intravenously (IV) with 1×10⁸ PFU of either SKV or FMT supplemented with either 10, 50 or 100 nmol AVT01 M05 MC38 either short (˜9mer) or long (˜25mer) peptides. Mice were boosted again on day 29 intravenously (IV) with 1×10⁸ PFU of MG1 supplemented with either 10, 50 or 100 nmol AVT01 M05 MC38 either short (˜9mer) or long (˜25mer) peptides. Blood was sampled on days 21 and 35. SKV refers to SKV refers to CopMD5p3p with a B8R gene deletion.

FIGS. 17A-17B. Cumulative immune response readout as intracellular IFN-γ expression in CD8+ T cells. FIG. 17A provides the experimental protocol for the results in FIG. 17B. FIG. 17B. shows the pooled sum of numbers of CD8+ T cells in peripheral blood expressing IFNγ in response to ex-vivo stimulation with individual minimal epitopes corresponding to neoantigens (MC38) used for vaccination. Mice were primed on day 1 with 10 nmol AVT01 M05 MC38 either short (˜9mer) or long (˜25mer) peptides. Mice were boosted on day 15 intravenously (IV) with 1×10⁸ PFU of either SKV or FMT supplemented with 50 nmol non-adjuvanted (no imidazoquinoline-based Toll-like receptor-7 and -8 agonist) AVT01 M05 MC38 either short (˜9mer) or long (˜25mer) peptides. Mice were boosted again on day 29 intravenously (IV) with 1×10⁸ PFU of MG1 supplemented with 50 nmol non-adjuvanted AVT01 M05 MC38 either short (˜9mer) or long (˜25mer) peptides. Blood was sampled on days 14, 22, 36 and 59.

FIGS. 18A-18B. Immune response readout of the Adpgk1 neo-antigen as intracellular IFN-γ expression in CD8+ T cells. FIG. 18A provides the experimental protocol for the results in FIG. 18B. FIG. 18B shows numbers of CD8+ T cells in peripheral blood expressing IFNγ in response to ex-vivo stimulation with Adpgk1 minimal epitope. Mice were either primed or not on days 1, 8 and 15 with AVT01 M05 MC38 long (˜25mer) peptides 10 nmol intramuscularly. Mice were boosted on day 29 intravenously (IV) with 1×10⁸ PFU of FMT-N10. Blood was sampled on days 28 and 35.

FIGS. 19A-19B. Immune response readout of the Adpgk1 and Cpne1 neo-antigens as intracellular IFN-γ expression in CD8+ T cells. FIG. 19A provides the experimental protocol for the results in FIG. 19B. FIG. 19B shows numbers of CD8+ T cells in peripheral blood expressing IFNγ in response to ex-vivo stimulation with either Adpgk1 or Cpne 1 minimal epitope. Mice were primed on day 1 with AVT01 M05 MC38 long (˜25mer) peptides at either 2.5 nmol, 10 nmol, 25 nmol, 50 nmol or 0 nmol (no prime). Mice were boosted on day 15 intravenously (IV) with 1×10⁸ PFU of FMT-N10. Blood was sampled on days 14 and 21.

FIGS. 20A-20B. Immune response readout of the Adpgk1 and Cpne1 neo-antigens as intracellular IFN-γ expression in CD8+ T cells. FIG. 20A provides the experimental protocol for the results in FIG. 20B. FIG. 20B shows numbers of CD8+ T cells in peripheral blood expressing IFNγ in response to ex-vivo stimulation with either Adpgk1 or Cpne 1 minimal epitope. Mice were primed on day 1 with 10 nmol intramuscularly of either M5 (AVT01 MC38 Adpgk, Irgq, Reps1, Cpne1 and Aatf), M2 (AVT01 MC38 Cpne1 and Aatf) and M3 (AVT01 MC38 Adpgk, Irgq and Reps1), M1 (AVT01 MC38 Adpgk) or M1 (AVT01 MC38 Adpgk) and AH1 (AVT01 AH1) long (˜25mer) peptides. Mice were boosted on day 15 intravenously (IV) with 1×10⁸ PFU of FMT-N10. Blood was sampled on days 14 and 21.

### 5. DETAILED DESCRIPTION

### 5.1 Neoantigens

In one aspect, provided herein are methods for inducing an immune response to one or more neoantigens. In a specific embodiment, neoantigens are mutated, non-self products that arise from some tumor accumulated genetic alterations. The inherent genetic instability of cancers can lead to mutations in DNA, RNA splice variants and changes in post-translational modification, which result in these de novo mutated, non-self protein products. These mutated protein products may be processed, presented by human leukocyte antigen (HLA) molecules and elicit T-cell responses to these tumor-specific somatic mutations. The mutated protein products are specific to tumor cells and are often but not always unique to an individual subject.

Generally, cancer patients have a tumor with a unique combination of neoantigens (sometimes referred to herein as “private neoantigens”). The term “mutanome” may be used herein to refer to the collective of a subject's tumor-specific mutations, which encode a set of neoantigens that are specific to the subject. See, e.g., Tureci et al., Clin Cancer Res. 2016; 22(8):1885-1896. The mutanome can readily be determined for a given tumor, e.g., by next generation sequencing.

In specific embodiments, a neoantigen is a tumor-associated antigen that is subject-specific, and is sometimes referred to herein as a “private neoantigen.” In other embodiments, a neoantigen appears across a patient population, and is sometimes referred to herein as a “public neoantigen.” For example, mutations that alter protein function to promote oncogenesis, so-called driver mutations, can systematically reappear across patients. See, e.g., Kiebanoff and Wolchok, 2017, J Exp. Med., 215(1):5-7. Non-limiting examples of public neoantigens include mutated KRAS, such as KRAS G12D (see, e.g., Tran et al., 2016, N. Engl. J. Med. 375: 225-2262) and KRAS G12V (see, e.g., Veatech et al., 2019, Cancer Immunol. Res. 7: 910-922), mutated p53, such as p53 p.R175H (see, e.g., Lo et al., 2019, Cancer Immunol. Res. 7: 534-543), and mutated histone, such as histone variant H3.3 (H3.3K27M) (see, e.g., Mackay et al., Cancer Cell 32: 520-537), and mutated calreticulin (see, e.g,. Bozkus et al., 2019, Cancer Discov. 9: 1-6). Public neoantigens may be used to develop targeted immunotherapy approaches applicable to significant patient populations in contrast to private neoantigens which generally require next generation sequencing and complex algorithms.

Neoantigens may arise from DNA mutations including, e.g., nonsynonymous missense mutations, nonsense mutations, insertions, deletions, chromosomal inversions and chromosomal translocations. Neoantigens may arise from RNA splice site changes or missense mutations that can introduce amino acids permissive to post-translational modifications (e.g., phosphorylation). In certain embodiments, neoantigens may be created by one, two, three or more of the following or a combination thereof: (1) nucleotide polymorphisms that result in non-conservative amino acid changes; (2) insertions and/or deletions, which can result in peptide antigens containing an insertion or deletion or a frameshift mutation; (3) the introduction of a stop codon that in its new context is not recognized by the stop codon machinery, resulting in the ribosome skipping the codon and generating a peptide that contains a single amino acid deletion; (4) mutations at splice sites, which result in incorrectly spliced mRNA transcripts; and (5) inversions and/or chromosomal translocations that result in fusion peptides.

In some embodiments, a process is used to select the one or more neoantigens to which to induce an immune response. Neoantigens may be prioritized according to their MHC binding affinity and RNA expression levels within tumor cells. For example, neoantigens may be prioritized according to their predicted MHC class I binding, their MHC class II binding, or both. See, e.g., Kreiter et al., 2015, Nature 520: 692-696 and Yadav et al., 2014, Nature 515: 572-578 for methods for predicting MHC binding of neoantigens. In some embodiments, additional criteria are applied, such as, e.g., predicted immunogenicity or predicted capacity of the neoantigen to lead to T cells that react with other self-antigens, which may lead to auto-immunity. In some embodiments, neoantigens that are predicted to result in T cell or antibody responses that react with self-antigens found on healthy cells are not selected for use in the methods described herein.

In a specific embodiment, a peptide or protein that is capable of inducing an immune response to a neoantigen is selected for use in a method described herein. The terms peptide or polypeptide may be used interchangeably herein to refer to a natural or non-natural amino acid sequence. The peptide or polypeptide may or may not contain post-translational modifications, such as, e.g., glycosylation, phosphorylation or both. As used herein, a peptide or protein that is capable of inducing an immune response to a neoantigen of interest may be referred to as an “antigenic protein,” whether in the context of a prime or a boost.

In some embodiments, a process is used to select a peptide or protein that is capable of inducing an immune response to one or more neoantigens. For example, the peptide or protein may be assessed for its MHC binding affinity, its structural similarity to a neoantigen, or both. In some embodiments, a peptide or protein is selected that is at least about 70% identical, at least about 80% identical, at least about 90% identical, at least about 95% identical to a particular neoantigen. In some embodiments, a peptide or protein is selected that is identical to a particular neoantigen. In some embodiments, a peptide or protein is selected that is structurally or conformationally similar to a particular neoantigen as assessed using a method to known to one of skill in the art, such as, e.g., NMR, X-ray crystaollographic methods, or secondary structure prediction methods, such as, e.g., circular dichroism. In a particular embodiment, a peptide or protein with the highest predicted MHC class I binding, MHC class II binding, or both may be selected to induce an immune response to one or more neoantigens. See, e.g., Kreiter et al., 2015, Nature 520: 692-696 and Yadav et al., 2014, Nature 515: 572-578 for methods for predicting MEW binding. In certain embodiments, a peptide or protein is selected for use in a method of inducing an immune response that is predicted to elicit a CD4 T cell response, a CD8 T cell response, or both. In some embodiments, a peptide or protein is selected for use in a method of inducing an immune response that contains a CD4 epitope. In certain embodiments, a peptide or protein is selected for use in a method of inducing an immune response that contains a CD8 epitope. In some embodiments, additional criteria are applied in the selection of a peptide or protein that is capable of inducing an immune response to a neoantigen, such as, e.g., predicted immunogenicity or predicted capacity of the peptide or protein to lead to T cells that react with other self-antigens, which may lead to auto-immunity. In some embodiments, peptides or proteins that are predicted to result in T cell or antibody responses that react with self-antigens found on healthy cells are not selected for use in the methods described herein.

The term “about,” as used herein refers to plus or minus 10% of a reference, e.g., a reference amount, time, length, or activity. In instances where integers are required or expected, it is understood that the scope of this term includes rounding up to the next integer and rounding down to the next integer. In instances where the reference is measured in terms of days, the scope of this term also includes plus or minus 1, 2, 3, or 4 days. For clarity, use herein of phrases such as “about X,” and “at least about X,” are understood to encompass and particularly recite “X.”

The determination of percent identity between two amino acid sequences may be accomplished using a mathematical algorithm. A non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the) (BLAST program of Altschul et al, 1990, J. Mol. Biol. 215:403. BLAST protein searches may be performed with the)(BLAST program parameters set, e.g., to score 50, word length=3 to obtain amino acid sequences homologous to a protein molecule described herein. To obtain gapped alignments for comparison purposes, Gapped BLAST may be utilized as described in Altschul et al, 1997, Nucleic Acids Res. 25:3389 3402. Alternatively, PSI BLAST may be used to perform an iterated search which detects distant relationships between molecules (Id.). When utilizing XBLAST, the default parameters of the program may be used (see, e.g., National Center for Biotechnology Information (NCBI), ncbi.nlm.nih.gov). Another non limiting example of a mathematical algorithm utilized for the comparison of sequences is the algorithm of Myers and Miller, 1988, CABIOS 4: 11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 may be used. The percent identity between two sequences may be determined using techniques similar to those described above, with or without allowing gaps. In calculating percent identity, typically only exact matches are counted.

In a specific embodiment, an antigenic protein that is identical to a neoantigen or a fragment thereof (e.g., a portion of the neoantigen that contains an epitope) is selected for use in the methods described herein. In one embodiment, the fragment of the neoantigen is at least 8 amino acids in length, and in some embodiments, the fragment is about 8 to about 15 amino acids in length, about 12 to about 15 amino acids in length, about 15 to about 25 amino acids in length, about 25 to 30 amino acids in length, about 25 to about 50 amino acids in length, about 25 to about 75 amino acids in length, or about 50 to about 75 amino acids in length. In some embodiments, the fragment of the neoantigen is about 50 to about 100 amino acids in length, about 75 to about 100 amino acids in length, about 75 to about 125 amino acids in length, about 100 to about 125 amino acids in length, about 125 to about 150 amino acids in length, about 100 to about 150 amino acids in length, about 150 to about 200 amino acids in length, about 8 to about 250 amino acids in length, or about 150 to about 300 amino acids in length. The antigenic protein that is used in the methods described herein may contain a CD4 epitope, a CD8 epitope, or both.

In certain embodiments, at least one antigenic protein of a composition (e.g., a priming composition, boosting composition, or both) containing one or more antigenic proteins ranges in length from about 8 to about 500 amino acids. For example, at least one antigenic protein may be at least about 8, at least about 10, at least about 20, at least about 25, at least about 30, at least about 40, at least about 50, at least about 100, at least about 200, at least about 250, at least about 300, or at least about 400 amino acids in length to about 500 amino acids in length. In other examples, at least one antigenic protein may be less than about 400, less than about 300, less than about 200, less than about 150, less than about 125, less than about 100, less than about 75, less than about 50, less than about 40, or less than about 30 amino acids to about 8 amino acids in length. Any combination of the stated upper and lower limits is also envisaged. In certain embodiments, at least one antigenic protein may be about 8, about 10, about 20, about 25, about 30, about 40, about 50, about 75, about 100, about 125, about 150, about 175, about 200, about 250, about 300, about 400, or about 500 amino acids in length. In some embodiments, one or more of the antigenic proteins may be synthetic proteins. In certain embodiments, one or more antigenic proteins may be recombinant proteins.

In certain embodiments, an antigenic protein is about 8 to about 500 amino acids in length, about 25 to about 500 amino acids in length, about 25 to about 400 amino acids in length, about 25 to about 300 amino acids in length, about 25 to about 200 amino acids in length, or about 25 to about 100 amino acids in length, and contains at least a fragment (e.g., an epitope) of at least one neoantigen of interest. In some embodiments, an antigenic protein is about 25 to about 250 amino acids in length, about 25 to about 75 amino acids in length, or about 25 to about 50 amino acids in length, and contains at least a fragment (e.g., an epitope) of at least one neoantigen of interest. In some embodiments, an antigenic protein about 250 to about 1000 amino acids in length, about 250 to about 750 amino acids in length, or about 250 to about 500 amino acids in length, and contains at least a fragment (e.g., an epitope) of at least one neoantigen of interest. Any combination of the stated upper and lower limits is also envisaged.

In certain embodiments, an antigenic protein that is used in a method of inducing an immune response described herein contains at least a fragment (e.g., an epitope) of one or more neoantigens of interest. Thus, in some embodiments, an antigenic protein that is used in a method of inducing an immune response described herein contains at least a fragment (e.g., an epitope) of 2, 3, 4, 5, 6, 7, 8, 9, 10 or more neoantigens of interest. In certain embodiments, an antigenic protein that is used in a method of inducing an immune response described herein contains at least a fragment (e.g., an epitope) of 2 to 20, 2 to 15, 2 to 10, 5 to 10, 15 to 20, or 2 to 5 neoantigens of interest. In some embodiments, the antigenic protein that is used in a method of inducing an immune response described herein contains at least two neoantigens or a fragment of each of the at least two neoantigens. In certain embodiments, the at least two neoantigens are public neoantigens. The appropriate combination of public neoantigens to be administered may be determined by a simple diagnostic test, such as, e.g., RT-PCR or through an ELISA immunoassay. In other embodiments, the at least two neoantigens are private neoantigens. In some embodiments, one of the least two neoantigens in a private neoantigen and the other of the least neoantigens is a public neoantigen. In other words, in some embodiments, an antigenic protein may comprises a mix of both public and private neoantigens.

In a specific embodiment, an antigenic protein is a fusion protein comprising 2 or more neoantigens or fragments (e.g., an epitope) of each of the 2 or more neoantigens. In certain embodiments, the fusion protein includes spacers, (e.g., proteosomal cleavage sites, such as, e.g., described in Section 6), or both. See, e.g., Schubert and Kohlbacher, 2016, Genome Medicine 8: 9 for techniques for designing antigenic proteins with optimal spacers.

In certain embodiments, an antigenic protein is a fusion protein comprising two or more neoantigens or fragments thereof, and the two neoantigens or fragments thereof are randomly ordered in the fusion protein. In some embodiments, an antigenic protein is a fusion protein comprising two or more neoantigens or fragments thereof, and the two neoantigens or fragments thereof are ordered 5′ to 3′ in the fusion protein on the basis of the predicted MHC binding affinity of the two or more neoantigens or fragments thereof. In certain embodiments, the neoantigen or fragment thereof with the highest predicted MHC binding affinity is first in the fusion protein. In other embodiments, the neoantigen or fragment thereof with the lowest predicted MHC binding affinity is last in the fusion protein. In a specific embodiment, a technique as described in Section 6, infra, is used to optimize the order of two or more neoantigens or fragments thereof in a fusion protein.

In a specific embodiment, an antigenic protein described herein is used to produce a peptide antigen conjugate. See, e.g., Section 5.2 for a description of peptide antigen conjugataes.

### 5.2 Peptide Antigen Conjugates

In one aspect, provided herein are particles comprising a peptide antigen conjugate that further comprises an antigenic protein (A) linked to a Particle (P) or hydrophobic molecule (H). Peptide antigen conjugate refers to the compound that results from linking, e.g., covalently joining or otherwise, the antigenic protein (A) to the Particle (P) or hydrophobic molecule (H). The hydrophobic molecule (H) or Particle (P) induces the antigenic protein conjugate to assemble into particles that leads to an unexpected improvement in immune responses directed against the antigenic protein (A). The peptide antigen conjugate may additionally comprise an optional N-terminal extension (B1) and/or C-terminal extension (B2) linked to the N- and C-termini of the antigenic protein (A), respectively that provide unexpected improvements in manufacturing and biological activity; an optional charged molecule (C) that provides unexpected improvements in the stability of particles formed by peptide antigen conjugates, thereby leading to improved manufacturing and improved biological activity; and an optional Linker (L) that results from the reaction of linker precursor XI linked to the antigenic protein (A) with the linker precursor X2 provided on the hydrophobic molecule (H) or Particle (P), thereby joining the antigenic protein (A) and hydrophobic molecule (H) and Particle (P) in an efficient process that leads to unexpected improvements in manufacturing efficiency of peptide antigen conjugates. The components comprising the peptide antigen conjugate may be linked through any suitable means and are described in greater detail in International Patent Application Publication No. WO 2018/187515 and U.S. Patent Application Publication No. 2020/0054741, each of which is incorporated by reference herein in its entirety. In a specific embodiment, an antigen peptide conjugate is one described in International Patent Application Publication No. WO 2018/187515 and U.S. Patent Application Publication No. 2020/0054741, each of which is incorporated by reference herein in its entirety.

The peptide antigen conjugate may comprise an antigenic protein (A), optional N- and/or C-terminal extensions (B1 and/or B2), optional Linker (L), Particle (P) or hydrophobic molecule (H) and optional charged molecule(s) (C). Each of these components are described below and in greater detail in International Patent Application Publication No. WO 2018/187515 and U.S. Patent Application Publication No. 2020/0054741, each of which is incorporated by reference herein in its entirety.

In the present disclosure, the term “hydrophobic molecule” (H) is used as a general term to describe a molecule with limited water solubility, or amphiphilic characteristics, that can be linked to antigenic proteins resulting in a peptide antigen conjugate that forms particles in aqueous conditions. The hydrophobic molecule (H) in this context promotes particle assembly due to its poor solubility, or tendency to assemble into particles, in aqueous conditions over certain temperatures and pH ranges.

Hydrophobic molecules (H) as described herein are inclusive of amphiphilic molecules that may form supramolecular structures, such as micelles or bilayer-forming lamellar or multi-lamellar structures (e.g., liposomes or polymer somes), as well as compounds that are completely insoluble and form aggregates alone. The hydrophobic characteristics of the molecule may be temperature- and/or pH-responsive. In some embodiments, the hydrophobic molecule (H) is a polymer that is water soluble at low temperatures but is insoluble, or micelle-forming, at temperatures above, for example, 20° C., such as 20, 21, 22, 23, 24, 25, 26, 27, 28, 29, 30, 31, 32, 33, 34, 35, 36, 37, 38, 39 or 40° C. In other embodiments, the hydrophobic molecule (H) is a polymer that is water soluble at low pH, for example, at a pH below 6.5 but insoluble, for example, at a pH above 6.5. Examples of hydrophobic molecules (H) include but are not limited to fatty acids, cholesterol and its derivatives, long chain aliphatics, lipids and various polymers, such as polystyrene, poly(lactic-co-glycolic acid) (PLGA), as well as poly(amino acids) comprised of predominantly hydrophobic amino acids. In some embodiments, the hydrophobic molecule (H) is a hydrophilic polymer with multiple hydrophobic ligands attached. A variety of hydrophobic molecules useful for the practice of the present disclosure are disclosed herein.

Charged molecule (C): A charged molecule (C) refers to any molecule that has one or more functional groups that are positively or negatively charged. The functional groups comprising the charged molecule may be partial or full integer values of charge. A charged molecule may be a molecule with a single charged functional group or multiple charged functional groups. Functional groups may be permanently charged or the functional groups comprising the charged molecule may have charge depending on the pH. The charged molecule may be comprised of positively charged functional groups, negatively charged functional groups or both positive and negatively charged functional groups. The net charge of the charged molecule may be positive, negative or neutral. The charge of a molecule can be readily estimated based on a molecule's Lewis structure and accepted methods known to those skilled in the art. Charge may result from inductive effects, e.g., atoms bonded together with differences in electron affinity may result in a polar covalent bond resulting in a partially negatively charged atom and a partially positively charged atom. For example, nitrogen bonded to hydrogen results in partial negative charge on nitrogen and a partial positive charge on the hydrogen atom. Alternatively, an atom may be considered to have a full integer value of charge when the number of electrons assigned to that atom is less than or equal to the atomic number of the atom. The charge of a functional group is determined by summing the charge of each atom comprising the functional group. The net charge of the charged molecule (C) is determined by summing the charge of each atom comprising the molecule. Those skilled in the art are familiar with the process of estimating charge of a molecule, or individual functional groups, by summing the formal charge of each atom in a molecule or functional group, respectively.

Linkers (L) are specific subsets of linkers that result from the reaction of the linker precursor XI with the linker precursor X2 and function specifically to join the antigenic protein (A) to a hydrophobic molecule (H) or Particle (P) either directly or indirectly through an extension (B 1 or B2) or charged molecule (C). Linkers perform the specific function of site-selectively coupling, i.e. joining or linking together the antigenic protein (A) with a hydrophobic molecule (H) or a Particle (P). A linker precursor XI may be linked to an antigenic protein directly or indirectly through an extension (B1 or B2) typically during solid-phase peptide synthesis. Note that the linker precursor XI linked directly to the N- or C-terminus of the antigenic protein (A) are not considered extensions as they do not specifically function to modulate the rate of degradation of the antigenic protein. While the linker precursor XI may have some impact on the rate of the degradation of the antigenic protein (A), the linker precursor XI is not selected to modulate the rate of degradation of the antigenic protein (A) or its release from other molecules and instead functions specifically to join the antigenic protein (A) to the hydrophobic molecule (H) or particle (P).

In some embodiments, a linker precursor XI can be linked to a antigenic protein (A) during solid phase peptide synthesis; the linkage can be direct, or indirect via an extension (B1 or B2), including a degradable peptide linker. Typically, the linker precursor XI linked directly or indirectly to the antigenic protein (A) is selected to promote a bio-orthogonal reaction with a linker precursor X2 provided on a hydrophobic molecule (H) or Particle (P). Bio-orthogonal reactions permit site-selective linkage of the antigenic protein (A) to the hydrophobic molecule (H) or Particle (P) without resulting in the modification of any amino acids comprising the antigenic protein (A). Preferred linker precursors XI that permit bio-orthogonal reactions include those bearing azides or alkynes. Additional linker precursors XI that permit site-selective reactivity, depending on the composition of the antigen, include thiols, hydrazines, ketones and aldehydes. In several embodiments, the linker precursor has an azide functional group. In some embodiments, the linker precursor XI is a non-natural amino acid bearing an azide, for example, azido-lysine Lys(N₃). In such embodiments, a antigenic protein (A) linked to the linker precursor XI bearing an azide functionality may react with an alkyne bearing linker precursor X2 provided on a hydrophobic molecule (H) resulting in the formation of a triazole Linker that joins the antigenic protein (A) and the hydrophobic molecule (H). Various linker precursors (XI and X2) and Linkers are described throughout in International Patent Application Publication Nos. WO 2018/187515 and WO 2019/226828, and U.S. Patent Application Publication No. 2020/0054741, each of which is incorporated by reference herein in its entirety.

Herein, the Linker and the linker precursor XI may both be referred to as a Tag (T), though, the context of the Tag (T) is used to discern whether the Tag (T) is a Linker or linker precursor (XI). A Tag (T) that is linked to a antigenic protein (A) either directly or indirectly through either the optional extension (B 1 or B2) or the optional charged molecule (C) but is not linked to a hydrophobic molecule (H) or Particle (P), may also be referred to as a linker precursor XI. A Tag (T) that links the antigenic protein (A) to a hydrophobic molecule (H) or Particle (P) may also be referred to as a Linker (L). The linker precursor X2 reacts with the linker precursor XI to form a Linker. The linker precursor XI may sometimes be referred to as a Tag and the linker precursor X2 may be referred to as a tag reactive moiety or tag reactive molecule comprising a functional group that is specific or reactive towards the Tag. Net charge: The sum of electrostatic charges carried by a molecule or, if specified, a section of a molecule.

Particle: A nano- or micro-sized supramolecular structure comprised of an assembly of molecules. Peptide antigen conjugates of the present disclosure comprise either antigenic proteins (A) linked to pre-formed Particles (P) or hydrophobic molecules (H) that assemble into micelles or other supramolecular structures. Particles comprising peptide antigen conjugates can be taken up into cells (e.g., immune cells, such as antigen-presenting cells). In some embodiments, the peptide antigen conjugate forms a particle in aqueous solution. In some embodiments, particle formation by the peptide antigen conjugate is dependent on pH or temperature. In some embodiments, the nanoparticles comprised of peptide antigen conjugates have an average diameter between 5 nanometers (nm) to 500 nm. In some embodiments, the nanoparticles comprised of peptide antigen conjugates may be larger than 100 nm. In some embodiments, the nanoparticles comprised of peptide antigen conjugates are included in larger particle structures that are too large for uptake by immune cells (e.g., particles larger than about 5000 nm) and slowly release the smaller nanoparticles comprising the peptide antigen conjugate

In some embodiments, the peptide antigen conjugates comprising a hydrophobic molecule (H) form nanoparticles. The nanoparticles form by association of peptide antigen conjugates through hydrophobic interactions and may therefore be considered a supramolecular assembly. In some embodiments, the nanoparticle is a micelle. In preferred embodiments, the nanoparticle micelles are between about 5 to 50 nm in diameter. In some embodiments, the peptide antigen conjugate forms micelles and the micelle formation is temperature-, pH- or both temperature- and pH-dependent. In some embodiments, the disclosed nanoparticles comprise peptide antigen conjugates that are comprised of antigenic proteins (A) linked to a hydrophobic molecule (H) comprised of polymers linked to a Ligand with adjuvant properties, e.g. a PRR agonist; linking the antigenic protein together with the PRR agonist in the nanoparticles prevents the PRR agonist from dispersing freely following administration to a subject thereby preventing systemic toxicity.

The particle may be formed by an assembly of individual molecules comprising the peptide antigen conjugates, or in the case of a peptide antigen conjugate comprised of a antigenic protein (A) linked to a pre-formed Particle (P), the particle may be cross-linked through covalent or non-covalent interactions.

Pre-formed Particle (P)/Particle (P) of a formula: The pre-formed Particle (P) or simply ‘Particle’ (P) describes a Particle that is already formed prior to linkage to a antigenic protein (A). Thus, Particle (P) is used to describe the Particle of a formula and is distinct from the particles formed by assembly of two or more peptide antigen conjugates comprising a hydrophobic molecule (H). For clarity, the particles formed by the assembly of peptide antigen conjugates are distinct from pre-formed Particles (P) or Particles (P) of a formula. In some embodiments, a antigenic protein (A) can be linked directly or indirectly to a Particle (P) to form a peptide antigen conjugate, and the peptide antigen conjugate can be a particle in aqueous conditions.

To delineate between particles formed by peptide antigen conjugates and pre-formed Particles (P), the letter p is always capitalized in ‘Particle’ followed by a parenthetical capital ‘P’, i.e., “Particle (P),” when referring to a pre-formed Particle or Particle (P) of a formula. In some embodiments, the Particle (P) may be a PLGA Particle (P) that is formed in aqueous conditions and then linked to a antigenic protein (A) to form a peptide antigen conjugate that remains as particles in aqueous conditions. In some embodiments, the Particle (P) may be comprised of lipids, such as a liposomal Particle (P), that is formed in aqueous conditions and then linked to antigenic proteins (A) to form a peptide antigen conjugate that remain as particles in aqueous conditions.

In some embodiments, the antigenic protein (A) is linked directly to a hydrophobic molecule (H) or Particle (P) to form a peptide antigen conjugate of the formula A-H or A-P. In other embodiments, the antigenic protein (A) is linked to a hydrophobic molecule (H) or Particle (P) through a Linker (L) to form a peptide antigen conjugate of the formula A-L-H or A-L-P. In still other embodiments, the antigenic protein (A) is linked to an extension (B1 or B2) that is linked either directly or through a Linker (L) to a hydrophobic molecule (H) or Particle (P) to form a peptide antigen conjugate of any one of the formulas, A-B2-H, A-B2-L-H, H-B1-A, H-L-B1-A, A-B2-P, A-B2-L-P, P-B1-A or P-L-B1-P. In some embodiments, the antigenic protein (A) is linked directly or through an extension (B1 and B2) to a linker precursor XI to form a antigenic protein fragment of the formula, A-X1, A-B2-X1, X1-A or X1-B1-A, that reacts with a linker precursor X2 on a hydrophobic molecule (H) or Particle (P), i.e. X2-H or X2-P, to form a Linker (L) that joins the antigenic protein (A) to the hydrophobic molecule (H) or Particle (P), resulting in a peptide antigen conjugate of any one of the formulas, i.e. A-L-H, A-L-P, A-B2-L-H, A-B2-L-P, H-L-A, P-L-A, H-B1-A, or P-B1-A. In the present disclosure, such embodiments are shown to form particles in aqueous conditions that are shown to be useful for inducing an immune response in a subject.

In some embodiments, the antigenic protein (A) is linked to both extensions (B1 and B2). Such embodiments include peptide antigen conjugates of the formula B1-A-B2-H, B1-A-B2-L-H, B1-A-B2-P, B1-A-B2-L-P, H-B1-A-B2, H-L-B1-A-B2, P-B 1-A-B2, or P-L-B1-A-B2. In the present disclosure, such embodiments are shown to form particles in aqueous conditions that are demonstrated to be useful for inducing an immune response in a subject.

In some embodiments, molecules that contain functional groups that impart electrostatic charge, i.e. charged molecules (C), are linked directly or indirectly through optional extensions (B1 and/or B2), the optional Linker (L) or the hydrophobic molecule (H) or Particle (P) to the antigenic protein (A). The charge imparted on the peptide antigen conjugate by the charged molecule stabilizes the supramolecular structures formed in aqueous conditions. Non-limiting examples of peptide antigen conjugates comprising charged molecules (C) include C-A-H, C-B1-A-H, C-A-B2-H, C-B1-A-B2-H, A-H(C), A-B2-H(C), B1-A-H(C), B1-A-B2-H(C), C1-A-H(C2), C1-A-B2-H(C2), C1-B1-A-H(C2), C1-B1-A-B2-H(C2), H-A-C, H-B 1-A-C, H-A-B2-C, H-B1-A-B2-C, H(C)-A, H(C)-B1-A, H(C)-A-B2, H(C)-B1-A-B2, H(C1)-A-C2, H(C1)-B1-A-C2, H(C1)-A-B2-C2, H(C1)-B1-A-B2-C2, C-A-L-H, C-B1-A-L-H, C-A-B2-L-H, C-B1-A-B2-L-H, A-L-H(C), A-B2-L-H(C), B1-A-L-H(C), B1-A-B2-L-H(C), C1-A-L-H(C2), C1-A-B2-L-H(C2), C1-B1-A-L-H(C2), C1-B1-A-B2-L-H(C2), H-L-A-C, H-L-B1-A-C, H-L-A-B2-C, H-L-B1-A-B2-C, H(C)-L-A, H(C)-L-B1-A, H(C)-L-A-B2, H(C)-L-B1-A-B2, H(C1)-L-A-C2, H(C1)-L-B1-A-C2, H(C1)-L-A-B2-C2, H(C1)-L-B1-A-B2-C2, C-A-P, C-B1-A-P, C-A-B2-P, C-B1-A-B2-P, A-P(C), A-B2-P(C), B1-A-P(C), B1-A-B2-P(C), C1-A-P(C2), C1-A-B2-P(C2), C1-B1-A-P(C2), C1-B1-A-B2-P(C2), P-A-C, P-B1-A-C, P-A-B2-C, P-B1-A-B2-C, P(C)-A, P(C)-B1-A, P(C)-A-B2, P(C)-B1-A-B2, P(C1)-A-C2, P(C1)-B1-A-C2, P(C1)-A-B2-C2, P(C1)-B1-A-B2-C2, C-A-L-P, C-B1-A-L-P, C-A-B2-L-P, C-B1-A-B2-L-P, A-L-P(C), A-B2-L-P(C), B l-A-L-P(C), B1-A-B2-L-P(C), C1-A-L-P(C2), C1-A-B2-L-P(C2), C1-B1-A-L-P(C2), C1-B 1-A-B2-L-P(C2), P-L-A-C, P-L-B1-A-C, P-L-A-B2-C, P-L-B 1-A-B2-C, P(C)-L-A, P(C)-L-B 1-A, P(C)-L-A-B2, P(C)-L-B1-A-B2, P(C1)-L-A-C2, P(C1)-L-B1-A-C2, P(C1)-L-A-B2-C2 or P(C1)-L-B1-A-B2-C2.

The charged molecule (C) stabilizes the particles formed by peptide antigen conjugates. The charged molecule (C) may be linked directly to the peptide antigen conjugate. Alternatively, the charged molecule (C) may be provided on a separate molecule that associates with the particles formed by peptide antigen conjugates. In some embodiments, the charged molecule (C) is linked to a hydrophobic molecule (H) to form a charged molecule conjugate of the formula C-H, or C-A′-H (wherein A′ is a conserved antigen), that is mixed with a peptide antigen conjugate of the formula [C]-[B 1]-A-[B2]-[L]-H, where [ ] denotes that the group is optional, in aqueous conditions and the resulting particles comprise C-H, or C-A′-H and the peptide antigen conjugate.

The hydrophobic molecule (H) may comprise any suitable molecule that induces the peptide antigen conjugate to assemble into particles in aqueous conditions. In some embodiments, the hydrophobic molecule (H) comprising a peptide antigen conjugate is a polymer with limited water solubility. In some embodiments, the hydrophobic molecule (H) is a temperature- or pH-responsive polymer that has limited water solubility at particular temperatures or pH values. In other embodiments, the hydrophobic molecule (H) is a lipid, fatty acid or cholesterol. Many hydrophobic molecules (H) are useful for the present disclosure and are described in greater detail throughout.

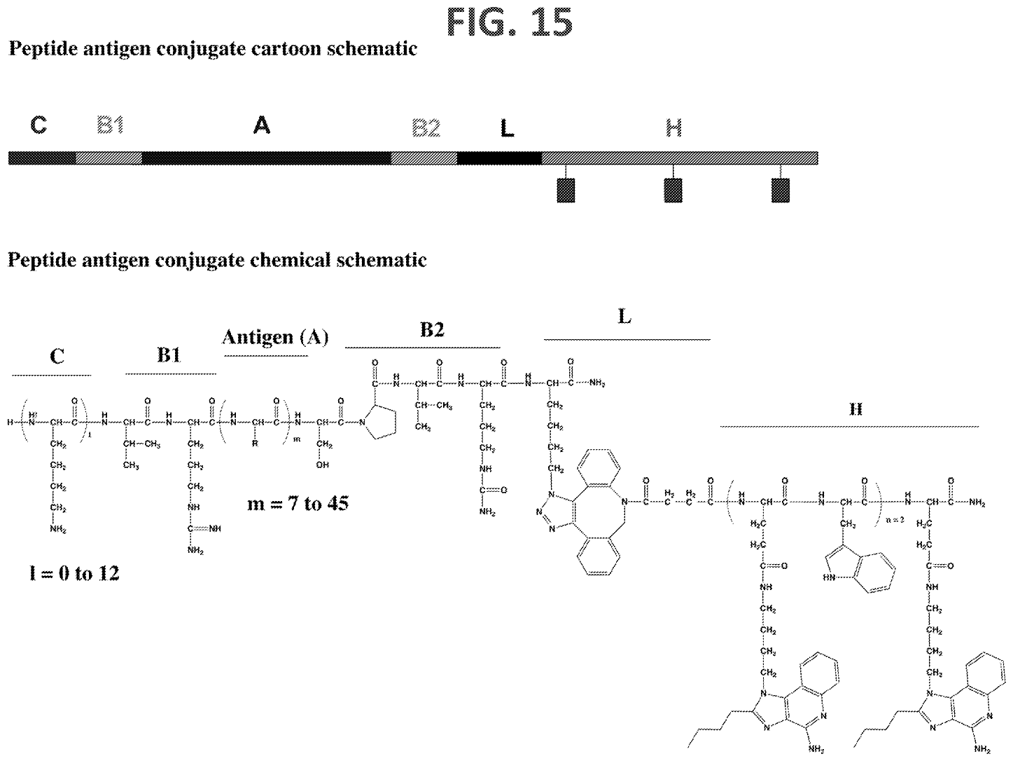

In some embodiments, a peptide antigen conjugate has the formula C-B1-A-B2-L-H (FIG. 15), wherein C is a charged molecule (sometimes referred to as a “charged moiety” or “charge modifying group”) consisting of multiple lysine residues that are positively charged at physiologic pH; B1 and B2 are N- and C-terminal extensions consisting of cathepsin degradable peptides, i.e. Val-Arg and Ser-Pro-Val-Cit, respectively; A is an antigenic protein; L is a linker, Lys(N3-DBCO), consisting of azido-lysine (Lys(N3), CAS #159610-92-1) linked to a dibenzylcyclooctyne (DBCO; CAS #: 1353016-70-2) through a triazole bond; and, H is a hydrophobic block (sometimes referred to as a “hydrophobic molecule”) consisting of an oligopeptide, Glu(2B)-Trp-Glu(2B)-Trp-Glu(2B)-NH2, wherein each Glutamic acid residue (Glu) is linked to an imidazoquinoline-based Toll-like receptor-7 and -8 agonist (TLR-7/8a). In a specific embodiment, the antigenic protein is one described herein (e.g., in Section 5.1). In another specific embodiment, the antigenic protein consists of between 7 to 45 amino acids comprising one or more minimal CD4 epitopes, CD8 T cell epitopes, or both. In another specific embodiment, the antigenic protein consists of between 9 to 35 amino acids comprising one or more minimal CD4 epitopes, CD8 T cell epitopes, or both.

Peptide antigen conjugates may be produced as described in International Patent Application Publication No. WO 2018/187515, U.S. Patent Application Publication No. 2020/0054741 or International Patent Application Publication No. WO 2019/226828, each of which is incorporated by reference herein in its entirety. In a specific embodiment, a peptide antigen conjugate is one described in International Patent Application Publication No. WO 2018/187515, U.S. Patent Application Publication No. 2020/0054741 or International Patent Application Publication No. WO 2019/226828, each of which is incorporated by reference herein in its entirety. In a specific embodiment, a peptide antigen conjugate is one described in Section 6, below.

### 5.3 Priming Compositions

In one aspect, provided herein are compositions for use as a prime in the methods presented herein. In a specific embodiment, provided herein are priming compositions that may be used in the methods presented herein. In a specific embodiment, a priming composition is capable of and is used to induce an immune response to one or more neoantigens in a subject. In certain embodiments, a priming composition is used to induce an immune response to 2 to about 20 neoantigens. In some embodiments, a priming composition is used to induce an immune response to 2, 3, 4, 5, 6, 7, 8, 9, or 10 neoantigens in a subject. In certain embodiments, a priming composition is used to induce an immune response to 1 to 3, 1 to 5, 2 to 4, 2 to 5, 2 to 6, 2 to 8, 5 to 8, 5 to 10, or 8 to 10 neoantigens in a subject. Any combination of the stated upper and lower limits is also envisaged. In a specific embodiment, a priming composition is one described in Section 6, infra, to prime a subject. In another embodiment, a priming composition comprises a peptide antigen conjugate described in Section 5.2, supra. In certain embodiments, in addition to a peptide antigen conjugate, a priming virus is used to prime a subject. The priming virus may be administered in the same or a different composition.

In one embodiment, a priming virus comprises a genome comprising a transgene, wherein the transgene encodes and expresses a protein in the subject, wherein the protein or a fragment thereof is capable of inducing an immune response to at least one neoantigen, and wherein the priming virus is immunologically distinct from an oncolytic virus used in a first boost of a method presented herein. In some embodiments, the priming virus is immunologically distinct from an oncolytic virus used in a first boost and a second boost of a method presented herein.

In certain embodiments, a priming virus is immunologically distinct from the oncolytic virus utilized in at least the first post-prime boost in a heterologous method described herein. In some embodiments, a priming virus is immunologically distinct from the oncolytic viruses utilized in each of the boosts in a heterologous boost method described herein.

In general, two viruses, e.g., two oncolytic viruses, are immunologically distinct when the two viruses do not induce neutralizing antibodies against each other to such a degree that the viruses may no longer deliver antigen to the immune system. In certain embodiments, two viruses, e.g., oncolytic viruses, are immunologically distinct when the viruses do not induce antibodies that substantially inhibit replication of the other as assessed by a virus neutralization assay, such as described in Tesfay et al., 2014, J. Virol. 88: 6148. In a specific embodiment, two viruses are immunologically distinct when one virus induces antibodies that inhibit the replication of the other virus in a virus neutralization assay, e.g., a virus neutralization assay described in Tesfay et al., 2014, J. Virol. 88: 6148, by less than about 0.5 logs, less than about 1 log, less than about 1.5 logs, or less than about 2 logs. Non-limiting examples of viruses that are immunologically distinct from each other include non-pseudotyped Farmington virus and Maraba virus (e.g., Maraba MG1 virus). Non-limiting examples of viruses wherein each is immunologically distinct from the other also include non-pseudotyped adenovirus, Farmington virus, Maraba virus (e.g., Maraba MG1 virus), vaccinia virus, and measles virus. Non-limiting examples of viruses wherein each is immunologically distinct from the other also include non-pseudotyped adenovirus, Farmington virus, vesicular stomatitis virus, vaccinia virus, and measles virus.

In some embodiments, a priming virus is an adenovirus. In certain embodiments, a priming virus is an oncolytic virus. See, e.g., Section 5.4 and 6, infra, for examples of oncolytic viruses. In some embodiments, the priming virus may be attenuated. For example, in certain embodiments, the priming virus may have reduced virulence, but still be viable or “live.” In specific embodiments, the primig virus is attenuated but replication-competent. In certain embodiments, the priming virus is replication-defective. In certain embodiments, a priming virus is inactivated, (e.g., UV inactivated).

In certain embodiments, a priming virus comprises a genome that comprises a transgene, wherein the transgene comprises a nucleic acid sequence that encodes an antigenic protein such that it is expressed in the subject. The transgene may also include additional sequences, such as, e.g., viral regulatory signals (e.g., gene end, intergenic, and/or gene start sequences) and Kozak sequences. Generally, the total length of a transgene is limited only by the nucleic acid carrying capacity of the particular virus, that is, the amount of nucleic acid that can be inserted into the genome of the virus without preventing a sufficient amount of the protein encoded by the transgene to be produced. In specific embodiments, a sufficient amount of the protein encoded by the transgene is enough to induce an immune response to a neoantigen. In certain embodiments, the total length of a transgene is limited only by the nucleic acid carrying capacity of the particular virus, that is, the amount of nucleic acid that can be inserted into the genome of the virus without significantly inhibiting the pre-insertion replication capability of the virus. In some embodiments, the amount of nucleic acid inserted into the genome of a virus does not significantly inhibit the pre-insertion replication capability of the virus if it does not reduce the replication by more than about 0.5 log, about 1 log, about 1.5 log, about 2 logs, about 2.5 logs, or about 3 logs in a particular cell line relative the replication of the virus absent the insert in the same cell line. In particular embodiments, for example, in instances where the virus is a Farmington virus or a Maraba virus, for example an MG1 virus, a transgene of about 3-5 kb, e.g., about 3 kb, about 3.5 kb, about 4 kb, about 4.5 kb, or about 5 kb, may be inserted into the virus genome. Techniques known in the art may be used to insert a transgene into the genome of a virus.

In certain embodiments where a priming composition comprises a priming virus that comprises a transgene, wherein the transgene encodes and expresses one or more antigenic proteins in a subject, at least one antigenic protein may range in length from about 8 to about 500 amino acids. In particular embodiments, at least one antigenic protein may be at least about 8, at least about 10, at least about 20, at least about 30, at least about 40, at least about 50, at least about 100, at least about 200, at least about 250, at least about 300, or at least about 400 amino acids in length to about 500 amino acids in length. In other examples, at least one antigenic protein may be less than about 400, less than about 300, less than about 200, less than about 150, less than about 125, less than about 100, less than about 75, less than about 50, less than about 40, or less than about 30 amino acids to about 8 amino acids in length. Any combination of the stated upper and lower limits is also envisaged. In certain embodiments, at least one antigenic protein may be about 8, about 10, about 20, about 25, about 30, about 40, about 50, about 75, about 100, about 125, about 150, about 175, about 200, about 250, about 300, about 400, or about 500 amino acids in length. In certain embodiments, each of the one or more antigenic proteins fall within these length parameters. In some embodiments, the transgene comprises a codon-optimized nucleotide sequence encoding an antigenic protein.

In instances where a transgene encodes and expresses one or more antigenic proteins in a subject, in certain embodiments, the transgene can express the more than one antigenic proteins as a single, longer protein. In instances wherein two or more antigenic proteins are expressed as part of a single, longer protein, in certain embodiments, the portion(s) of the longer protein corresponding to at least one individual antigenic protein fall(s) within these length parameters. In other embodiments, the portions of the longer protein corresponding to each of the individual antigenic proteins fall within these length parameters.

In certain embodiments where a transgene encodes and expresses an antigenic protein in a subject, the antigenic protein may comprise the entire amino acid sequence of the neoantigen of interest. In such embodiments, the antigenic protein may be as long or longer than the neoantigen of interest.

In some embodiments, a priming virus comprises a genome that comprises transgene or nucleic acid sequences, wherein the transgene or nucleic acid sequences express×number of antigenic proteins, the virus may comprise a nucleic acid for each of the antigenic proteins, that is, a first nucleic acid that expresses the first antigenic protein, a second nucleic acid that expresses the second antigenic protein, etc., up to and including an xth nucleic acid that encodes the xth antigenic protein. In particular embodiments, the first antigenic protein is capable of inducing an immune response to a first neoantigen, the second antigenic protein is capable of inducing an immune response to a second neoantigen, etc., up to and including the xth antigenic protein being capable of inducing an immune response to an xth neoantigen. In certain embodiments, the transgene or nucleic acid sequences that express x number of antigenic proteins does not prevent a sufficient amount of the protein encoded by the transgene to be produced. In specific embodiments, a sufficient amount of the protein encoded by the transgene is enough to induce an immune response to the xth neoantigen. In a specific embodiment, the transgene or nucleic acid sequences that express x number of antigenic proteins does not significantly inhibit the pre-insertion replication capability of the virus if the transgene or nucleic acid sequence inserted into the viral genome does not reduce the replication of the virus by more than about 0.5 log, about 1 log, about 1.5 log, about 2 logs, about 2.5 logs, or about 3 logs in a particular cell line relative the replication of the virus absent the insert in the same cell line.

Within the virus, a nucleic acid sequence that expresses a particular antigenic protein may be contiguous to or separate from a nucleic acid sequence that expresses a different antigenic protein. In certain embodiments, each of the nucleic acid sequences expressing the antigenic protein may be present in the virus as a transgene. In some embodiments, each of the nucleic acid sequences expressing antigenic proteins is a fusion protein. As noted above, generally, the total length or lengths of such nucleic acid or nucleic acid sequences within the virus need only be limited by the nucleic acid carrying capacity of the virus. In certain embodiments, the nucleic acid sequences may express antigenic proteins as individual proteins. In certain embodiments, nucleic acid sequences may express antigenic proteins together as part of a longer protein. In certain embodiments, nucleic acid sequences may express certain of antigenic proteins as individual proteins and certain of antigenic proteins together as part of a longer protein. In instances where two or more antigenic proteins are expressed as part of a longer protein, the antigenic proteins may be adjacent to each other, with no intervening amino acids between them, or may be separated by an amino acid spacer. In certain embodiments involving a longer protein, some of antigenic proteins may be adjacent to each other and others may be separated by an amino acid spacer. In certain embodiments, the longer protein comprises one or more cleavage sites, for example, one or more proteasomal cleavage sites. In particular embodiments, the protein comprises one or more amino acid spacers that comprise one or more cleavage sites, for example, one or more proteasomal cleavage sites. See, e.g., Section 6, infra, for examples of nucleic acid sequences encoding one or more antigenic proteins.

In some embodiments, a priming composition comprises a peptide antigen conjugate containing one or more peptides capable of inducing an immune response to a first subset of the neoantigens of interest, and a priming virus that comprises a genome comprising a transgene(s) or nucleic acid sequence(s), wherein the transgene(s) or nucleic acid sequence(s) express one or more proteins capable of inducing an immune response to a second subset of the neoantigens of interest. In some embodiments, the first subset includes public neoantigens of interest and the second subset includes private neoantigens, or vice versa. In some embodiments, the first subset and second subset each include private neoantigens or public neoantigens. In certain embodiments, the first subset and the second subset of neoantigens of interest are overlapping subsets. In other embodiments, the first subset and the second subset of neoantigens of interest do not overlap. In some embodiments, the peptide antigen conjugate and the priming virus are administered in the same composition. In other embodiments, the peptide antigen conjugate and the priming virus are administered in different compositions. The different compositions may be formulated for administration by the same or a different route of administration.

In some embodiments, a priming virus does not comprise a genome that comprises a nucleic acid sequence or transgene that expresses an antigenic protein. A virus that does not comprise a genome that comprises nucleic acid sequence or transgene that expresses the antigenic protein refers to a virus that does not produce the antigenic protein and does not cause a cell infected by the virus to produce the protein. For example, the priming virus may lack a nucleic acid sequence that encodes the amino acid sequence of the antigenic protein, or lack nucleic acid sequences necessary for the transcription and/or translation required for the virus to express the antigenic protein or to cause a cell infected by the virus to express the antigenic protein. In another example, the priming virus may lack a nucleic acid sequence that encodes the amino acid sequence of the antigenic protein, and lack nucleic acid sequences necessary for the transcription and/or translation required for the virus to express the antigenic protein or to cause a cell infected by the virus to express the antigenic protein. In one embodiment, a priming virus that does not comprise a genome that comprises a transgene or a nucleic acid sequence that expresses the antigenic protein is an adenovirus (e.g., an adenovirus of serotype 5). For example, in one embodiment, an adenovirus is a recombinant replication-incompetent human Adenovirus serotype 5.

In certain embodiments, a priming virus that does not comprise a genome that comprises a transgene or nucleic acid sequence that expresses an antigenic protein may be attenuated. For example, in certain embodiments, the virus of the prime may have reduced virulence, but still be viable or “live.” In certain embodiments, a priming virus that does not comprise a genome that comprises a transgene or nucleic acid sequence that expresses an antigenic protein is replication-defective. In some embodiments, a priming virus that does not comprise a genome that comprises a transgene or nucleic acid sequence that expresses an antigenic protein is inactivated, (e.g., UV inactivated).

In a particular embodiment, a priming virus is not engineered to (i) contain a transgene or nucleic acid sequence that encodes the amino acid sequence of the antigenic protein, or (ii) contain nucleic acid sequences necessary for the transcription and/or translation required for the virus to express the antigenic protein or to cause a cell infected by the virus to express the antigenic protein. In another embodiment, a priming virus is not engineered to (i) contain a transgene or nucleic acid sequence that encodes the amino acid sequence of the antigenic protein, and (ii) contain nucleic acid sequences necessary for the transcription and/or translation required for the virus to express the antigenic protein or to cause a cell infected by the virus to express the antigenic protein.

In certain embodiments, a priming composition described herein further comprises an adjuvant. In certain embodiments, the adjuvant can potentiate an immune response to an antigen or modulate it toward a desired immune response. In some embodiments, the adjuvant can potentiate an immune response to an antigen and modulate it toward a desired immune response. In one embodiment, the adjuvant is polyI:C.

In one embodiment, a priming composition is formulated for intravenous, intramuscular, subcutaneous, intraperitoneal or intratumoral administration. When a priming composition is to be administered in parts, different parts of the priming composition may be formulated for the same or different routes of administration. For example, when a priming composition comprises a first composition and a second composition, wherein the first composition comprises a priming virus, and the second composition comprises an peptide antigen conjugate, the first composition may be administered by the same or a different route than the second composition. In a particular embodiment, a priming composition is formulated for intravenous administration. In another embodiment, a priming composition is formulated for subcutaneous or intramuscular administration.

In certain embodiments, a priming composition comprises 1×10⁷ to 5×10¹² PFU of a priming virus. For example, in some embodiments, a priming composition comprises 1×10⁷ to 1×10¹² PFU of a priming virus. In certain embodiments, a priming composition comprises about 1×10¹¹ PFU, about 2×10¹¹ PFU, or a dose described in Section 6. In some embodiments, a priming composition comprises about 10 μg to about 1000 μg one or more antigenic proteins. In certain embodiments, a priming composition comprises about 10 μg to about 1000 μg one or more nucleic acid sequences encoding one or more antigenic proteins.

In certain embodiments, a priming composition further comprises an immune-potentiating compound such as cyclophosphamide (CPA).

### 5.4 Boost Compositions

In one aspect, provided herein are boost compositions or compositions for a boost that may be used in the methods presented herein. In a specific embodiment, a boost composition is used to induce an immune response to one or more neoantigens in a subject. In certain embodiments, a boost composition is used to induce an immune response to 2 to about 20 neoantigens. In some embodiments, a boost composition is used to induce an immune response to 2, 3, 4, 5, 6, 7, 8, 9, or 10 neoantigens in a subject. In certain embodiments, a boost composition is used to induce an immune response to 1 to 3, 1 to 5, 2 to 4, 2 to 5, 2 to 6, 2 to 8, 5 to 8, 5 to 10, or 8 to 10 neoantigens in a subject. Any combination of the stated upper and lower limits is also envisaged. In a specific embodiment, a boost composition is one described in Section 6, infra, or compositions similar to those described in Section 6, infra, with different neoantigens.

Generally, the methods presented herein utilize one or more boosts that comprise an oncolytic virus. Without wishing to be bound by theory or mechanism, an oncolytic virus may act as an adjuvant in a boost composition. By “oncolytic virus” is meant any one of a number of viruses that have been shown, when active, to specifically replicate and kill tumour cells in vitro or in vivo. These viruses may naturally be oncolytic viruses, or the viruses may have been modified to produce or improve oncolytic activity. In certain embodiments the term may encompass attenuated, replication defective, inactivated, engineered, or otherwise modified forms of an oncolytic virus suited to purpose.

In certain aspects, the methods presented herein utilize boosts that comprise a virus that is replication-competent and exhibits local replication in a subject, that is, replicates in only a subset of cell types in the subject, wherein the replication does not put the subject at risk. For example, the virus may replicate in immune organs (e.g., one or more lymph nodes, spleen or both), tumour cells, or both immune organs and tumor cells. While for ease of description, the methods and boost compositions presented herein generally refer to oncolytic viruses, it is understood that such methods and compositions can utilize and comprise such a virus.

In one embodiment, the oncolytic virus is attenuated. In one embodiment, the oncolytic virus exhibits reduced virulence relative to wild-type virus, but is still replication-competent. In one embodiment, the oncolytic virus is replication defective. In one embodiment, the oncolytic virus is inactivated (e.g., is UV inactivated).

In one embodiment, an oncolytic virus is a Rhabdovirus. “Rhabdovirus” include, inter alia, one or more of the following viruses or variants thereof: Carajas virus, Chandipura virus, Cocal virus, Isfahan virus, Piry virus, Vesicular stomatitis Alagoas virus, BeAn 157575 virus, Boteke virus, Calchaqui virus, Eel virus American, Gray Lodge virus, Jurona virus, Klamath virus, Kwatta virus, La Joya virus, Malpais Spring virus, Mount Elgon bat virus, Perinet virus, Tupaia virus, Farmington, Bahia Grande virus, Muir Springs virus, Reed Ranch virus, Hart Park virus, Flanders virus, Kamese virus, Mosqueiro virus, Mossuril virus, Barur virus, Fukuoka virus, Kern Canyon virus, Nkolbisson virus, Le Dantec virus, Keuraliba virus, Connecticut virus, New Minto virus, Sawgrass virus, Chaco virus, Sena Madureira virus, Timbo virus, Almpiwar virus, Aruac virus, Bangoran virus, Bimbo virus, Bivens Arm virus, Blue crab virus, Charleville virus, Coastal Plains virus, DakArK 7292 virus, Entamoeba virus, Garba virus, Gossas virus, Humpty Doo virus, Joinjakaka virus, Kannamangalam virus, Kolongo virus, Koolpinyah virus, Kotonkon virus, Landjia virus, Maraba virus, Manitoba virus, Marco virus, Nasoule virus, Navarro virus, Ngaingan virus, Oak-Vale virus, Obodhiang virus, Oita virus, Ouango virus, Parry Creek virus, Rio Grande cichlid virus, Sandjimba virus, Sigma virus, Sripur virus, Sweetwater Branch virus, Tibrogargan virus, Xiburema virus, Yata virus, Rhode Island, Adelaide River virus, Berrimah virus, Kimberley virus, or Bovine ephemeral fever virus. In certain aspects, a Rhabdovirus may refer to the supergroup of Dimarhabdovirus (defined as rhabdovirus capable of infecting both insect and mammalian cells).

In a particular embodiment, the Rhabdovirus is a Farmington virus or an engineered variant thereof. For exemplary, non-limiting examples of nucleotide sequences of the Farmington virus genome see GenBank Accession Nos. KC602379.1 (Farmington virus strain CT114); and HM627182.1. As is well-known, rhabdoviruses are negative-strand RNA viruses. As such, it is understood that nucleotide sequences of their genomes can include RNA and reverse complement versions of these representative sequences.

In another particular embodiment, the Rhabdovirus is a Maraba virus or an engineered variant thereof. In one embodiment, for example, the oncolytic virus is an attenuated Maraba virus comprising a Maraba G protein in which amino acid 242 is mutated, and a Maraba M protein in which amino acid 123 is mutated. In one embodiment, amino acid 242 of the G protein is arginine (Q242R), and the amino acid 123 of the M protein is tryptophan (L123W). An example of the Maraba M protein is described in PCT Application No. PCT/M2010/003396 and U.S Patent Application Publication No. US2015/0275185, which are incorporated herein by reference, wherein it is referred to as SEQ ID NO: 4. An example of the Maraba G protein is described in PCT Application No. PCT/M2010/003396 and U.S Patent Application Publication No. US2015/0275185, wherein it is referred to as SEQ ID NO: 5. In one embodiment, the oncolytic virus is the Maraba double mutant (“Maraba DM”) described in PCT Application No. PCT/M2010/003396 and U.S Patent Application Publication No. US2015/0275185. In one embodiment, the oncolytic virus is the “Maraba MG1” described in PCT Application No. PCT/CA2014/050118; U.S. patent Ser. No. 10/363,293; and U.S Patent Application Publication No. US2019/0240301, which are incorporated herein by reference. As used herein, Maraba MG1 may be referred to as “MG1 virus.”

In another particular embodiment, the Rhabdovirus is a Farmington virus or an engineered variant thereof. In one embodiment, the oncolytic virus is a Farmington virus described in PCT Application No. PCT/CA2012/050385, U.S. Patent Application Publication No. US2016/028796514 and PCT/CA2019/050433.

In one embodiment, the oncolytic virus is a vaccinia virus, measles virus, or a vesicular stomatitis virus.

In certain embodiments, the oncolytic virus is a vaccinia virus, e.g., a Copenhagen (see, e.g., GenBank M35027.1), Western Reserve, Wyeth, Lister (see, e.g., GenBank KX061501.1; DQ121394.1), EM63, ACAM2000, LC16m8, CV-1, modified vaccinia Ankara (MV A), Dairen I, GLV-1h68, IE1D-J, L-IVP, LC16m8, LC16mO, Tashkent, Tian Tan (see, e.g., AF095689.1), or WAU86/88-1 virus (for representative, non-limiting examples of nucleotide sequences, see the GenBank Accession Nos. provided in parentheses). In one embodiment, the vaccinia virus is a vaccinia virus with one or more beneficial mutations and/or one or more gene deletions or gene inactivations. For example, in certain embodiments, the vaccinia virus is a CopMD5p, CopMD3p, or CopMD5p3p vaccinia virus as described in WO 2019/134049, which is incorporated herein by reference in its entirety, and in particular for its description of these vaccinia viruses. In some embodiments, the vaccinia virus is SKV, which is CopMD5p3p vaccinia virus with a B8R gene deletion.

In one embodiment, the virus is an oncolytic adenovirus, e.g., an adenovirus comprising a deletion in E1 and E3, which renders the adenovirus susceptible to p53 inactivation. Because many tumours lack p53, such a modification effectively renders the virus tumour-specific, and hence oncolytic. In one embodiment, the adenovirus is of serotype 5.

In one embodiment, a boost comprises an oncolytic virus that comprises a genome comprising a transgene, wherein the transgene encodes and expresses a protein in a subject, wherein the protein or a fragment thereof is capable of inducing an immune response to at least one neoantigen, and wherein the oncolytic virus is immunologically distinct from an oncolytic virus used in a subsequent boost of a method presented herein. In some embodiments, the oncolytic virus is immunologically distinct from an oncolytic used in a boost and a subsequent boost of a method presented herein.

In certain embodiments, an oncolytic virus is immunologically distinct from the oncolytic virus utilized in at least the first post-prime boost in a heterologous method described herein. In some embodiments, an oncolytic virus is immunologically distinct from the oncolytic viruses utilized in each of the boosts in a heterologous boost method described herein.

In another embodiment, a boost comprises an oncolytic virus and a peptide, wherein the peptide or fragment thereof is capable of inducing an immune response to at least one neoantigen, that is an antigenic protein, and wherein the oncolytic virus is immunologically distinct from an oncolytic virus used in at least the immediately subsequent boost. The oncolytic virus and peptide may be formulated in one composition or different compositions. A composition comprising the oncolytic virus and a composition comprising the peptide may be formulated for the same route or different routes of administration to a subject. In some embodiments, the oncolytic virus is immunologically distinct from an oncolytic used in each of the boosts of a method presented herein. In certain embodiments, the oncolytic virus comprises a genome that comprises a transgene or a nucleic acid sequence that expresses an antigenic protein.

In another embodiment, a boost comprises a first composition and a second composition, wherein the first composition comprises an oncolytic virus, and the second composition comprises a peptide, wherein the peptide or fragment thereof is capable of inducing an immune response to at least one neoantigen, that is an antigenic protein, and wherein the oncolytic virus is immunologically distinct from an oncolytic virus used in at least the immediately subsequent boost. The first composition and second composition may be formulated for the same or a different route of administration to a subject. In some embodiments, the oncolytic virus is immunologically distinct from an oncolytic used in each of the boosts of a method presented herein. In certain embodiments, the oncolytic virus comprises a genome that comprises a transgene or a nucleic acid sequence that expresses an antigenic protein.

In some embodiments, a boost may comprise (i) one or more peptides capable of inducing an immune response to the one or more neoantigens of interest, that is, may comprise one or more antigenic proteins, and (ii) an oncolytic virus that comprises a genome comprising a transgene(s) or nucleic acid sequence(s), wherein the transgene(s) or nucleic acid sequence(s) express one or more proteins capable of inducing an immune response to the one or more neoantigens of interest, that is, express one or more antigenic proteins. In particular embodiments, a boost comprises one or more peptides capable of inducing an immune response to a first subset of the neoantigens of interest, and an oncolytic virus that comprises a genome comprising a transgene(s) or nucleic acid sequence(s), wherein the transgene(s) or nucleic acid sequence(s) express one or more proteins capable of inducing an immune response to a second subset of the neoantigens of interest. In certain embodiments, the first subset and the second subset of neoantigens of interest are overlapping subsets. In other embodiments, the first subset and the second subset of neoantigens of interest do not overlap. In certain embodiments, the first subset of neoantigens are public neoantigens and the second subset are private neoantigens, or vice versa. In some embodiments, the first and second subsets are private or public neoantigens. In certain embodiments, a boost comprises (i) one or more peptides capable of inducing an immune response to the neoantigens of interest, and (ii) an oncolytic virus comprises a genome that comprises a transgene(s) or nucleic acid sequence(s), wherein the transgene(s) or nucleic acid sequence(s) expresses one or more proteins capable of inducing an immune response to the neoantigens of interest. In some embodiments, the one or more peptides and the oncolytic virus are administered in the same composition. In other embodiments, the one or more peptides and the oncolytic virus are administered in different compositions. The different compositions may be formulated for administration by the same or a different route of administration.

In some embodiments, a boost may comprise (i) one or more peptides capable of inducing an immune response to the one or more neoantigens of interest, that is, may comprise one or more antigenic proteins, and (ii) an oncolytic virus that does not comprise a genome that comprises a nucleic acid sequence or transgene that expresses an antigenic protein. A virus that does not comprise a genome that comprises nucleic acid sequence or transgene that expresses the antigenic protein refers to a virus that does not produce the antigenic protein and does not cause a cell infected by the virus to produce the protein. For example, the oncolytic virus may lack a nucleic acid sequence that encodes the amino acid sequence of the antigenic protein, or lack nucleic acid sequences necessary for the transcription and/or translation required for the virus to express the antigenic protein or to cause a cell infected by the virus to express the antigenic protein. In another example, the oncolytic virus may lack a nucleic acid sequence that encodes the amino acid sequence of the antigenic protein, and lack nucleic acid sequences necessary for the transcription and/or translation required for the virus to express the antigenic protein or to cause a cell infected by the virus to express the antigenic protein.

In a particular embodiment, an oncolytic virus is not engineered to (i) contain a transgene or nucleic acid sequence that encodes the amino acid sequence of the antigenic protein, or (ii) contain nucleic acid sequences necessary for the transcription and/or translation required for the virus to express the antigenic protein or to cause a cell infected by the virus to express the antigenic protein. In another embodiment, an oncolytic virus is not engineered to (i) contain a transgene or nucleic acid sequence that encodes the amino acid sequence of the antigenic protein, and (ii) contain nucleic acid sequences necessary for the transcription and/or translation required for the virus to express the antigenic protein or to cause a cell infected by the virus to express the antigenic protein.

In certain embodiments, an oncolytic virus that does not comprise a transgene or nucleic acid sequence that expresses the antigenic protein, the antigenic protein is not physically associated with and/or connected to the virus. For example, in certain embodiments, the antigenic protein (i) is not attached to, conjugated to or otherwise covalent bonded to the oncolytic virus, (ii) does not become attached to, conjugated to or otherwise covalenty bonded to the oncolytic virus, (iii) does not non-covalently interact with the oncolytic virus, or (iv) does not form non-covalent interactions with the oncolytic virus. In some embodiments, two, three or all of the following apply to the antigenic protein: (i) the antigenic protein is not attached to, conjugated to or otherwise covalent bonded to the oncolytic virus, (ii) the antigenic protein does not become attached to, conjugated to or otherwise covalenty bonded to the oncolytic virus, (iii) the antigenic protein does not non-covalently interact with the oncolytic virus, and (iv) the antigenic protein does not form non-covalent interactions with the oncolytic virus. In other particular embodiments, the antigenic protein is may be physically associated with and/or connected to the virus. For example, in particular embodiments, the antigenic protein (i) may be attached to, conjugated to or otherwise covalent bonded to the virus, (ii) may become attached to, conjugated to or otherwise covalenty bonded to the virus, (iii) may non-covalently interact with the virus, or (iv) form non-covalent interactions with the virus. In some embodiments, one, two, three or all of the following apply to the antigenic protein: (i) may be attached to, conjugated to or otherwise covalent bonded to the virus, (ii) may become attached to, conjugated to or otherwise covalenty bonded to the virus, (iii) may non-covalently interact with the virus, and (iv) form non-covalent interactions with the virus.

In certain embodiments, a boost comprises one or more antigenic proteins. In some embodiments, a boost comprises one or more antigenic proteins, wherein at least one antigenic protein ranges in length from about 8 to about 500 amino acids. For example, at least one antigenic protein may be at least about 8, at least about 10, at least about 20, at least about 25, at least about 30, at least about 40, at least about 50, at least about 100, at least about 200, at least about 250, at least about 300, or at least about 400 amino acids in length to about 500 amino acids in length. In other examples, at least one antigenic protein may be less than about 400, less than about 300, less than about 200, less than about 150, less than about 125, less than about 100, less than about 75, less than about 50, less than about 40, or less than about 30 amino acids to about 8 amino acids in length. Any combination of the stated upper and lower limits is also envisaged. In certain embodiments, at least one antigenic protein may be about 8, about 10, about 20, about 25, about 30, about 40, about 50, about 75, about 100, about 125, about 150, about 175, about 200, about 250, about 300, about 400, or about 500 amino acids in length. In certain embodiments, one or more of the antigenic proteins of a boost may be synthetic proteins. In some embodiments, one or more antigenic proteins of a boost may be recombinant proteins.

In certain embodiments, a boost comprises an antigenic protein, wherein the antigenic protein may comprise the entire amino acid sequence of the neoantigen of interest. In such embodiments, the antigenic protein may be as long or longer than the neoantigen of interest. In some embodiments, a boost comprises an antigenic protein, wherein antigenic protein may comprise an amino acid sequence shorter than the neoantigen of interest, but a minimum of about 8 amino acid residues, about 9 amino acid residues, about 10 amino acid residues, about 11 amino acid residues, or about 12 amino acid residues in length.

In certain embodiments in which a boost comprises an oncolytic virus that comprises a genome comprising a transgene, the transgene comprises a nucleic acid sequence that encodes an antigenic protein such that it is expressed in the subject. The transgene may also include additional sequences, such as, e.g., viral regulatory signals (e.g., gene end, intergenic, and/or gene start sequences) and Kozak sequences. Generally, the total length of a transgene is limited only by the nucleic acid carrying capacity of the particular virus, that is, the amount of nucleic acid that can be inserted into the genome of the virus without preventing a sufficient amount of the protein encoded by the transgene to be produced. In specific embodiments, a sufficient amount of the protein encoded by the transgene is enough to induce an immune response to a neoantigen. In certain embodiments, the total length of a transgene is limited only by the nucleic acid carrying capacity of the particular virus, that is, the amount of nucleic acid that can be inserted into the genome of the virus without significantly inhibiting the pre-insertion replication capability of the virus. In some embodiments, the amount of nucleic acid inserted into the genome of a virus does not significantly inhibit the pre-insertion replication capability of the virus if it does not reduce the replication by more than about 0.5 log, about 1 log, about 1.5 log, about 2 logs, about 2.5 logs, or about 3 logs in a particular cell line relative the replication of the virus absent the insert in the same cell line. In particular embodiments, for example, in instances where the virus is a Farmington virus or a Maraba virus, for example an MG1 virus, a transgene of about 3-5 kb, e.g., about 3 kb, about 3.5 kb, about 4 kb, about 4.5 kb, or about 5 kb, may be inserted into the virus genome. In the case of Maraba virus, e.g., MG1 virus, the nucleic acids expressing the antigenic proteins may, for example, be inserted into the Maraba genome between the G and L gene sequences. In the case of Farmington virus, e.g., FMT virus, the nucleic acids expressing the antigenic proteins may, for example, be inserted into the Farmington genome between the N and P gene sequences. Techniques known in the art may be used to insert a transgene into the genome of a virus.

In certain embodiments where a boost comprises an oncolytic virus that comprises a genome that comprises transgene, wherein the transgene that encodes and expresses one or more antigenic proteins in a subject, at least one antigenic protein may range in length from about 8 to about 500 amino acids. In particular embodiments, at least one antigenic protein may be at least about 8, at least about 10, at least about 20, at least about 30, at least about 40, at least about 50, at least about 100, at least about 200, at least about 250, at least about 300, or at least about 400 amino acids in length to about 500 amino acids in length. In other examples, at least one antigenic protein may be less than about 400, less than about 300, less than about 200, less than about 150, less than about 125, less than about 100, less than about 75, less than about 50, less than about 40, or less than about 30 amino acids to about 8 amino acids in length. Any combination of the stated upper and lower limits is also envisaged. In certain embodiments, at least one antigenic protein may be about 8, about 10, about 20, about 25, about 30, about 40, about 50, about 75, about 100, about 125, about 150, about 175, about 200, about 250, about 300, about 400, or about 500 amino acids in length. In certain embodiments, each of the one or more antigenic proteins fall within these length parameters.

In instances where a transgene encodes and expresses one or more antigenic proteins in a subject, in certain embodiments, the transgene can express the more than one antigenic protein as a single, longer protein. In instances wherein two or more antigenic proteins are expressed as part of a single, longer protein, in certain embodiments, the portion(s) of the longer protein corresponding to at least one individual antigenic protein fall(s) within these length parameters. In other embodiments, the portions of the longer protein corresponding to each of the individual antigenic proteins fall within these length parameters.

In certain embodiments where a transgene encodes and expresses an antigenic protein in a subject, the antigenic protein may comprise the entire amino acid sequence of the neoantigen of interest. In such embodiments, the antigenic protein may be as long or longer than the neoantigen of interest.

In some embodiments, an oncolytic virus comprises a genome that comprises transgene or nucleic acid sequences, wherein the transgene or nucleic acid sequences express x number of antigenic proteins, the virus may comprise a nucleic acid for each of the antigenic proteins, that is, a first nucleic acid that expresses the first antigenic protein, a second nucleic acid that expresses the second antigenic protein, etc., up to and including an xth nucleic acid that encodes the xth antigenic protein. In particular embodiments, the first antigenic protein is capable of inducing an immune response to a first neoantigen, the second antigenic protein is capable of inducing an immune response to a second neoantigen, etc., up to and including the xth antigenic protein being capable of inducing an immune response to an xth neoantigen. In certain embodiments, the transgene or nucleic acid sequences that express x number of antigenic proteins does not prevent a sufficient amount of the protein encoded by the transgene to be produced. In specific embodiments, a sufficient amount of the protein encoded by the transgene is enough to induce an immune response to the xth neoantigen. In a specific embodiment, the transgene or nucleic acid sequences that express x number of antigenic proteins does not significantly inhibit the pre-insertion replication capability of the virus if the transgene or nucleic acid sequence inserted into the viral genome does not reduce the replication of the virus by more than about 0.5 log, about 1 log, about 1.5 log, about 2 logs, about 2.5 logs, or about 3 logs in a particular cell line relative the replication of the virus absent the insert in the same cell line

Within the virus, a nucleic acid sequence that expresses a particular antigenic protein may be contiguous to or separate from a nucleic acid sequence that expresses a different antigenic protein. In certain embodiments, each of the nucleic acid sequences expressing the antigenic protein may be present in the virus as a transgene. In some embodiments, each of the nucleic acid sequences expressing antigenic proteins may be present in the virus as a fusion protein. As noted above, generally, the total length or lengths of such nucleic acid or nucleic acid sequences within the virus need only be limited by the nucleic acid carrying capacity of the virus. In certain embodiments, the nucleic acid sequences may express antigenic proteins as individual proteins. In certain embodiments, nucleic acid sequences may express antigenic proteins together as part of a longer protein. In certain embodiments, nucleic acid sequences may express certain of antigenic proteins as individual proteins and certain of antigenic proteins together as part of a longer protein. In instances where two or more antigenic proteins are expressed as part of a longer protein, the antigenic proteins may be adjacent to each other, with no intervening amino acids between them, or may be separated by an amino acid spacer. In certain embodiments involving a longer protein, some of antigenic proteins may be adjacent to each other and others may be separated by an amino acid spacer. In certain embodiments, the longer protein comprises one or more cleavage sites, for example, one or more proteasomal cleavage sites. In particular embodiments, the protein comprises one or more amino acid spacers that comprise one or more cleavage sites, for example, one or more proteasomal cleavage sites. See, e.g., Section 6, infra, for examples of nucleic acid sequences encoding one or more antigenic proteins.

In some embodiments, an antigenic protein, a nucleic acid sequence expressing an antigenic protein, or a priming virus, is not encapsulated in a delivery vehicle such as a liposomal preparation or nanoparticle. In a specific embodiment, an antigenic protein is not encapsulated in a delivery vehicle such as a liposomal preparation or nanoparticle. In another embodiment, a nucleic acid sequence expressing an antigenic protein is not encapsulated in a delivery vehicle such as a liposomal preparation or nanoparticle. In another embodiment, a priming virus is not encapsulated in a delivery vehicle such as a liposomal preparation or nanoparticle.

In certain embodiments, a boost described herein further comprises an adjuvant. In certain embodiments, the adjuvant can potentiate an immune response to an antigen or modulate it toward a desired immune response. In some embodiments, the adjuvant can potentiate an immune response to an antigen and modulate it toward a desired immune response. In one embodiment, the adjuvant is polyI:C.

In some embodiments, an antigenic protein or an oncolytic virus is not encapsulated in a delivery vehicle such as a liposomal preparation or nanoparticle. In a specific embodiment, an antigenic protein is not encapsulated in a delivery vehicle such as a liposomal preparation or nanoparticle. In another embodiment, an oncolytic virus is not encapsulated in a delivery vehicle such as a liposomal preparation or nanoparticle. In another embodiment, an oncolytic virus and antigenic protein is not encapsulated in a delivery vehicle such as a liposomal preparation or nanoparticle.

In some embodiments, a boost described herein further comprises a liposome(s) or a nanoparticle(s). In a specific embodiment, liposomes (such as, e.g., N-[1-(2,3-dioleoloxy)propyl]-N,N,N-trimethyl ammonium chloride 1(DOTAP)) or nanoparticles may be used to wrap or encapsulate an antigenic protein or an oncolytic virus, or both. See, e.g., Sahin et al. (2014), mRNA-based therapeutics developing a new class of drugs. NATURE REVIEWS DRUG DISCOVERY, 13(10):759-780; Su et al. (2011) In vitro and in vivo mRNA delivery using lipid-enveloped pH-responsive polymer nanoparticles, MOLECULAR PHARMACEUTICALS, 8(3):-774-778; Phua et al., (2014) Messenger RNA (mRNA) nanoparticle tumour vaccination, NANOSCALE, 6(14):7715-7729; Bockzkowski et al., Dendritic cells pulsed with RNA are potent antigen-presenting cells in vitro and in vivo, JOURNAL OF EXPERIMENTAL MEDICINE, 184(2):465-472.

In some embodiments, a boost described herein further comprises a liposome(s) or a nanoparticle(s) and an adjuvant. In a specific embodiment, liposomes (such as, e.g., N-[1-(2,3-dioleoloxy)propyl]-N,N,N-trimethyl ammonium chloride 1(DOTAP)) or nanoparticles may be used to wrap or encapsulate (1) an antigenic protein, (2) an oncolytic virus and (3) an adjuvant. In another specific embodiment, liposomes (such as, e.g., N-[1-(2,3-dioleoloxy)propyl]-N,N,N-trimethyl ammonium chloride 1(DOTAP)) or nanoparticles may be used to wrap or encapsulate (1) an antigenic protein or an oncolytic virus and (2) an adjuvant.

In some embodiments, a boost comprises a peptide antigen conjugate as well as an oncolytic virus. The peptide antigen conjugate and oncolytic virus may be administered in the same or different compositions. See Section 5.2 above for peptide antigen conjugates that may be used.

In one embodiment, a boost is formulated for intravenous, intramuscular, subcutaneous, intraperitoneal or intratumoral administration. When a boost is to be administered in parts, different parts of the boost may be formulated for the same or different routes of administration. For example, when a boost comprises a first composition and a second composition, wherein the first composition comprises an oncolytic virus, and the second composition comprises an antigenic protein, the first composition may be administered by the same or a different route than the second composition. In a particular embodiment, a boost is formulated for intravenous administration. In another embodiment, a boost is formulated for subcutaneous or intramuscular administration.

In certain embodiments, a boosting composition comprises 1×10′ to 5×10¹² PFU of an oncolytic virus. For example, in some embodiments, a boosting composition comprises 1×10′ to 1×10¹² PFU of an oncolytic virus. In certain embodiments, a boosting composition comprises about 1×10¹¹ PFU, about 2×10¹¹ PFU, or a dose described in Section 6. In some embodiments, a boosting composition comprises about 10 μg to about 1000 μg one or more antigenic proteins.

In certain embodiments, a boost further comprises an immune-potentiating compound such as cyclophosphamide (CPA).

### 5.5 Methods of Inducing an Immune Response to Neoantigens

In one aspect, provided herein are methods for inducing an immune response to one or more neoantigens in a subject, comprising administering a dose of a priming composition and subsequently administering at least one boost. In a specific embodiment, provided herein are methods of inducing an immune response to one or more neoantigens in a subject, comprising administering a prime and one or more boosts. For example, in certain embodiments, such a methods induce an immune response to 2 to about 20 neoantigens, e.g., 2 to about 10 neoantigens, 2-5 neoantigens, for example 2, 3, 4 or 5 neoantigens. The priming composition may be one described in Section 5.3 or 6. The boost may comprise at least one boosting composition described in Section 5.4 or 6. In some embodiments, the methods involve administering multiple doses of a priming composition. In certain embodiments, the methods involve administering two sequential heterologous boosts. For example, the methods involve administering a priming composition described in Section 5.3 and two boosting compositions described in Section 5.4.

The term “subject,” as used herein, refers to a mammal, for example, a non-human mammal, a primate, e.g., a non-human primate, or a human. In one embodiment, a subject is a human subject. In certain embodiments, a subject has a pre-existing immunity to a neoantigen of interest. In certain embodiments, a subject is naïve with respect to immunity to a neoantigen of interest. In specific embodiments, a subject has cancer or has been diagnosed as having cancer.

In another aspect, provided herein are sequential heterologouos boost methods designed to induce an immune response to one or more neoantigens of interest. For example, in certain embodiments, such sequential heterologous boost methods induce an immune response to 2 to about 20 neoantigens, e.g., 2 to about 10 neoantigens, 2-5 neoantigens, for example 2, 3, 4 or 5 neoantigens. The sequential heterologous boost methods presented herein utilize oncolytic virus-comprising boosts wherein any two consecutive boosts utilize oncolytic viruses that are immunologically distinct from each other. Boosts that utilize oncolytic viruses that are immunologically distinct from each other may be referred to herein as heterologous boosts. The sequential heterologous boost methods presented herein may, for example, utilize any of the antigenic proteins, priming compositions and/or boost compositions described herein.

In certain embodiments, a sequential heterologous boost method as presented herein is a method of inducing an immune response to one or more neoantigens of interest in a subject, wherein the subject has a pre-existing immunity to the one or more neoantigens of interest. In certain embodiments, a sequential heterologous boost method as presented herein is a method of inducing an immune response to one or more neoantigens of interest in a subject, wherein the subject is naïve with respect to immunity to the one or more neoantigens of interest.

In particular embodiments, a sequential heterologous boost method as presented herein is a method of inducing an immune response to one or more neoantigens of interest in a subject, wherein the subject has been identified as having a pre-existing immunity to the one or more neoantigens of interest, and wherein the method comprises administering to the subject at least one consecutive heterologous boost, such that an immune reaction to the one or more neoantigens of interest. In certain embodiments, the method comprises administering to the subject a dose of a priming composition prior to boosting.

In other particular embodiments, a sequential heterologous boost method as presented herein is a method of inducing an immune response to one or more neoantigens in a subject, wherein the method comprises determining whether a subject has a pre-existing immunity to the one or more neoantigens of interest, and subsequently administering to the subject at least one sequential heterologous boost, such that an immune response to the one or more neoantigens is induced. For example, determining whether a subject has a pre-existing immunity to the one or more neoantigens of interest may comprise determining whether the subject contains CD8+ T cells specific for the one or more neoantigens of interest, e.g., determining whether peripheral blood from the subject contains antigen-specific interferon gamma positive CD8+ T cells. In embodiments where a subject is determined to have a preexisting immunity, the method further comprises administering to the subject at least one consecutive heterologous boost, such that an immune reaction to the one or more neoantigens of interest is induced, and may, in certain embodiments, comprise administering to the subject a dose of a priming composition prior to boosting.

In certain embodiments, a sequential heterologous boost method as presented herein is a method of inducing an immune response to one or more neoantigens of interest in a subject, wherein the subject is naïve with respect to immunity to the one or more neoantigens of interest. In certain embodiments, a sequential heterologous boost method as presented herein is a method of inducing an immune response to one or more neoantigens of interest, in a subject, wherein the subject is one that has been identified as naïve with respect to immunity to the one or more neoantigens of interest, and wherein the method comprises administering to the subject a dose of a priming composition and, subsequently, at least one pair of consecutive heterologous boosts such that an immune response to the neoantigen or neoantigens is induced.

In certain embodiments, a sequential heterologous boost method as presented herein is a method of inducing an immune response to one or more neoantigens of interest in a subject, wherein the method comprises determining whether a subject is naïve with respect to immunity to the one or more neoantigens of interest, and subsequently administering to the subject a dose of a priming composition that induces an immune response to the neoantigen or neoantigens, and subsequent to the administration of the priming composition, administering to the subject at least one pair of consecutive heterologous boosts such that an immune response to the neoantigen or neoantigens is induced. For example, determining whether a subject is naïve with respect to immunity to the one or more neoantigens of interest may comprise determining whether the subject contains CD8+ T cells specific for the one or more neoantigens of interest, e.g., determining whether peripheral blood from the subject contains antigen-specific interferon gamma positive CD8+ T cells.

With respect to inducing an immune response to at least one neoantigen, it will be appreciated that the at least one antigenic protein of the priming composition and the at least one antigenic protein of the boost(s) (or antigenic protein(s) expressed by a nucleic acid(s) of the oncolytic viruses of any of the boost(s), as appropriate) need not be exactly the same in order to accomplish this. Likewise, it will be appreciated that the at least one antigenic protein of any of the boosts (or the antigenic protein(s) expressed by a nucleic acid(s) of the oncolytic viruses of any of the boost(s), as appropriate) need not be exactly the same in order to accomplish this. For example, the proteins may comprise sequences that partially overlap, with the overlapping segment(s) comprising a sequence corresponding to a sequence of the neoantigen, or a sequence designed to induce an immune reaction to the neoantigen, thereby allowing an effective prime and boosts to the neoantigen to be achieved. For instance, the proteins may comprise sequences that partially overlap, with the overlapping segment(s) comprising a sequence corresponding to a sequence of the neoantigen, or a sequence designed to induce an immune reaction to the neoantigen, thereby allowing an effective prime and boosts to the neoantigen to be achieved. For example, the proteins may both share a sequence that comprises at least one epitope of the neoantigen. In another example, the proteins may comprise sequences that partially overlap, with the overlapping segment(s) comprising a sequence corresponding to the sequence of the neoantigen.

For a particular neoantigen, for example, in one embodiment the sequence of the antigenic protein of the priming composition and the sequence of the antigenic protein of any of the boosts (or the protein expressed by a nucleic acid of an oncolytic virus of any of the boosts) are at least about 70% identical, at least about 80% identical, at least about 90% identical, at least about 95% identical, or are identical. In another embodiment, the sequence of the antigenic protein of the priming composition and the sequence of the antigenic protein of each of the boosts (or the antigenic protein expressed by a nucleic acid of an oncolytic virus of each of the boosts) are at least about 70% identical, at least about 80% identical, at least about 90% identical, at least about 95% identical, or are identical.

For a particular neoantigen, in one embodiment, for example, the sequence of the antigenic protein of each of the boosts (or the antigenic protein expressed by a nucleic acid of an oncolytic virus of each of the boosts) are identical. In another such embodiment, for example, the sequence of the antigenic protein of the priming composition (or the antigenic protein expressed by a nucleic acid of a virus contained in the priming composition), and the sequence of the antigenic protein of each of the boosts (or the protein expressed by a nucleic acid of an oncolytic virus of each of the boosts) are identical.

In additional embodiments, for a particular neoantigen, the sequence of the antigenic protein of the priming composition and the sequence of the protein of each of the boosts (or the antigenic protein expressed by a nucleic acid of an oncolytic virus of each of the boosts) are at least about 70% identical, at least about 80% identical, at least about 90% identical, at least about 95% identical, or are identical, and the sequence of the antigenic protein of each of the boosts (or the antigenic protein expressed by a nucleic acid of an oncolytic virus of each of the boosts) are at least about 70% identical, at least about 80% identical, at least about 90% identical, at least about 95% identical, or are identical to each other. In another embodiment, the sequence of the antigenic protein of the priming composition and the sequence of the antigenic protein of each of the boosts (or the antigenic protein expressed by a nucleic acid of an oncolytic virus of each of the boosts) are at least about 70% identical, at least about 80% identical, at least about 90% identical, at least about 95% identical, or are identical, and the sequence of the antigenic protein of any of the boosts (or the antigenic protein expressed by a nucleic acid of an oncolytic virus of any of the boosts) are at least about 70% identical, at least about 80% identical, at least about 90% identical, at least about 95% identical, or are identical to each other.

In further embodiments, for a particular neoantigen, the sequence of the protein of the priming composition and the sequence of the antigenic protein of any of the boosts (or the antigenic protein expressed by a nucleic acid of an oncolytic virus of any of the boosts) are at least about 70% identical, at least about 80% identical, at least about 90% identical, at least about 95% identical, or are identical, and the sequence of the protein of each of the boosts (or the antigenic protein expressed by a nucleic acid of an oncolytic virus of each of the boosts) are at least about 70% identical, at least about 80% identical, at least about 90% identical, at least about 95% identical, or are identical to each other. In another embodiment, the sequence of the antigenic protein of the priming composition and the sequence of the antigenic protein of any of the boosts (or the antigenic protein expressed by a nucleic acid of an oncolytic virus of any of the boosts) are at least about 70% identical, at least about 80% identical, at least about 90% identical, at least about 95% identical, or are identical, and the sequence of the antigenic protein of any of the boosts (or the antigenic protein expressed by a nucleic acid of an oncolytic virus of any of the boosts) are at least about 70% identical, at least about 80% identical, at least about 90% identical, at least about 95% identical, or are identical to each other.

In specific embodiments, for a particular neoantigen, for example, in one embodiment the sequence of the antigenic protein of the priming composition and the sequence of the antigenic protein of any of the boosts (or the protein expressed by a nucleic acid of an oncolytic virus of any of the boosts) are identical over a contiguous stretch of about 70%, about 80%, about 90% or 95% of either protein. In another embodiment, the sequence of the antigenic protein of the priming composition and the sequence of the antigenic protein of each of the boosts (or the antigenic protein expressed by a nucleic acid of an oncolytic virus of each of the boosts) are identical over a contiguous stretch of about 70%, about 80%, about 90% or 95% of either protein.

In additional specific embodiments, for a particular neoantigen, the sequence of the antigenic protein of the priming composition and the sequence of the antigenic protein of each of the boosts (or the antigenic protein expressed by a nucleic acid of an oncolytic virus of each of the boosts) are identical over a contiguous stretch of about 70%, about 80%, about 90% or 95% of either protein, and the sequence of the antigenic protein of each of the boosts (or the antigenic protein expressed by a nucleic acid of an oncolytic virus of each of the boosts) are identical over a contiguous stretch of about 70%, about 80%, about 90% or 95% of each other. In another embodiment, the sequence of the antigenic protein of the priming composition and the sequence of the antigenic protein of each of the boosts (or the antigenic protein expressed by a nucleic acid of an oncolytic virus of each of the boosts) are identical over a contiguous stretch of about 70%, about 80%, about 90% or 95% of either antigenic protein, and the sequence of the antigenic protein of any of the boosts (or the antigenic protein expressed by a nucleic acid of an oncolytic virus of any of the boosts) are identical over a contiguous stretch of about 70%, about 80%, about 90% or 95% of each other.

In further specific embodiments, for a particular neoantigen, the sequence of the antigenic protein of the priming composition and the sequence of the antigenic protein of any of the boosts (or the antigenic protein expressed by a nucleic acid of an oncolytic virus of any of the boosts) are identical over a contiguous stretch of about 70%, about 80%, about 90% or 95% of either protein, and the sequence of the antigenic protein of each of the boosts (or the antigenic protein expressed by a nucleic acid of an oncolytic virus of each of the boosts) are identical over a contiguous stretch of about 70%, about 80%, about 90% or 95% of each other. In another embodiment, the sequence of the antigenic protein of the priming composition and the sequence of the antigenic protein of any of the boosts (or the antigenic protein expressed by a nucleic acid of an oncolytic virus of any of the boosts) are identical over a contiguous stretch of about 70%, about 80%, about 90% or 95% of either antigenic protein, and the sequence of the antigenic protein of any of the boosts (or the antigenic protein expressed by a nucleic acid of an oncolytic virus of any of the boosts) are identical over a contiguous stretch of about 70%, about 80%, about 90% or 95% of each other.

The population of at least two antigenic proteins from the prime and the population of at least two antigenic proteins from the boost may have complete, partial or no overlap in identity. In various embodiments, the at least two antigenic proteins of the prime and the boost are identical. In various embodiments, none of the at least two antigenic proteins of the prime and the boost are identical. In various embodiments, at least one of the at least two antigenic proteins from the first administration are identical to at least one of the at least two antigenic proteins from the second administration.

Utilization of one or more heterologous boosts may impart a substantially beneficial effect on the magnitude and/or duration of the resulting immune response, e.g., the CD8+ T cell response. The immune response may, for example, be measured by determining the absolute number of neoantigen-specific CD8+ T cells, for example, the number of antigen-specific interferon gamma (IFN-γ)-positive CD8+ T cells per ml of peripheral blood from the subject. See, e.g., Section 6, infra, and and Pol et al. “Maraba virus as a potent oncolytic vaccine vector.” Molecular therapy: the journal of the American Society of Gene Therapy vol. 22, 2 (2014): 420-429. doi:10.1038/mt.2013.249 for an example of a method for assessing the immune response induced by one or more heterologous boosts.

In certain embodiments of a sequential heterologous boost method presented herein, for a pair of consecutive heterologous boosts, e.g., the first and second consecutive heterologous boosts of the method, the peak immune response to a neoantigen of interest that is induced in a subject after administration of the second boost of the pair is equal to or higher than the peak immune response to the neoantigen induced by administration of the first boost in the pair. For example, in certain embodiments of a sequential heterologous boost method presented herein, for a pair of consecutive heterologous boosts, e.g., the first and second consecutive boosts of the method, the peak immune response to a neoantigen of interest that is induced in a subject after administration of the second boost of the pair comprises a peak immune response to the neoantigen that is at least about 0.1 log, about 0.2 log, about 0.3 log, about 0.4 log, about 0.5 log, about 0.75 log, about 1.0 log, about 1.2 log, about 1.5 log, or about 2.0 log higher than the peak immune response to the neoantigen induced by administration of first boost in the pair. The immune response may, for example, be measured by determining the absolute number of antigen-specific CD8+ T cells, for example, the number of antigen-specific interferon gamma (IFN-γ)-positive CD8+ T cells per ml of peripheral blood from the subject. See, e.g., Section 6, infra, for an example of a method for assessing the immune response induced by one or more heterologous boosts. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect my be observed with respect to the aggregate immune response to the neoantigens of interest.

In certain embodiments of a sequential heterologous boost method presented herein, for a pair of consecutive heterologous boosts, e.g., the first and second consecutive heterologous boosts of the method, with respect to the immune response to a neoantigen of interest induced in a subject by administration of the second boost of the pair, for at least one week, two weeks, three weeks, four weeks, one month, two months or three months after administration of the second boost the immune response attained to the neoantigen remains equal to or higher than the peak immune response to the antigen induced with administration of first boost in the pair. The immune response may, for example, be measured by determining the percentage of neoantigen-specific CD8+ T cells (for example, the number of neoantigen-specific interferon gamma (IFN-γ)-positive CD8+ T cells) of total CD8+ T cells per ml of peripheral blood from the subject. See, e.g., Section 6, infra, for an example of a method for assessing the immune response induced by one or more heterologous boosts. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect my be observed with respect to the aggregate immune response to the neoantigens of interest.

In certain embodiments of a sequential heterologous boost method presented herein, for a pair of consecutive heterologous boosts, e.g., the first and second consecutive heterologous boosts of the method, 1) the peak immune response to a neoantigen of interest that is induced in a subject after administration of the second boost of the pair is equal to or higher than the peak immune response to the neoantigen induced by administration of the first boost in the pair; and 2) with respect to the immune response to a neoantigen of interest induced in a subject by administration of the second boost of the pair, for at least one week, two weeks, three weeks, four weeks, one month, two months or three months after administration of the second boost the immune response attained to the neoantigen remains equal to or higher than the peak immune response to the antigen induced with administration of first boost in the pair. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect my be observed with respect to the aggregate immune response to the neoantigens of interest.

In certain embodiments of a sequential heterologous boost method presented herein, for a pair of consecutive heterologous boosts, e.g., the first and second consecutive boosts of the method, 1) the peak immune response to a neoantigen of interest that is induced in a subject after administration of the second boost of the pair comprises a peak immune response to the neoantigen that is at least about 0.1 log, about 0.2 log, about 0.3 log, about 0.4 log, about 0.5 log, about 0.75 log, about 1.0 log, about 1.2 log, about 1.5 log, or about 2.0 log higher than the peak immune response to the neoantigen induced by administration of first boost in the pair; and 2) with respect to the immune response to a neoantigen of interest induced in a subject by administration of the second boost of the pair, for at least one week, two weeks, three weeks, 4 weeks, one month, two months or three months after administration of the second boost the immune response attained to the neoantigen remains equal to or higher than the peak immune response to the antigen induced with administration of first boost in the pair. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect my be observed with respect to the aggregate immune response to the neoantigens of interest.

In certain embodiments of a sequential heterologous boost method presented herein, for a pair of consecutive heterologous boosts, e.g., the first and second consecutive boosts of the method, 1) the peak immune response to a neoantigen of interest that is induced in a subject after administration of the second boost of the pair comprises a peak immune response to the neoantigen that is at least about 0.1 log, about 0.2 log, about 0.3 log, about 0.4 log, about 0.5 log higher than the peak immune response to the antigen induced by administration of first boost in the pair; and 2) with respect to the immune response to a neoantigen of interest induced in a subject by administration of the second boost of the pair, for at least one month after administration of the second boost the immune response attained to the antigen remains equal to or higher than the peak immune response to the neoantigen induced with administration of first boost in the pair. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect my be observed with respect to the aggregate immune response to the neoantigens of interest.

In certain embodiments of a sequential heterologous boost method presented herein, for a pair of consecutive heterologous boosts, e.g., the first and second consecutive boosts of the method, increase the immune response to each neoantigen of interest is increased following the second boost. In instances where the sequential heterologous boost method is a method that induces an immune response to at least two neoantigens of interest in a subject, such an effect may be observed with respect to the immune response induced to at least one neoantigen of interest. In other instances where the sequential heterologous boost method is a method of inducing an immune response to at least two neoantigens of interest in a subject, such an effect my be observed with respect to the aggregate immune response to the neoantigens of interest.

In certain embodiments of a sequential heterologous boost method presented herein, for a pair of consecutive heterologous boosts, e.g., the first and second consecutive boosts of the method, the antigen-specific CD8+ T cells in peripheral blood following the latter boost comprises T effector cells (T_{eff} cells) and T effector memory cells (Tₑₘ cells), and the majority of such cells do not exhibit an “exhausted” T cell phenotype. For example, in particular embodiments, less than about 15%, less than about 20%, less than about 30%, less than about 40% or less than about 50% of antigen-specific T_{eff} cells and/or Tₑₘ cells are positive for PD-1, CTLA-4, and LAG-3. In other particular embodiments, less than about 15%, less than about 20%, less than about 30%, less than about 40% or less than about 50% of antigen-specific T_{eff} cells and Tₑₘ cells are positive for PD-1, CTLA-4, and LAG-3. In yet other particular embodiments, less than about 15%, less than about 20%, less than about 30%, less than about 40% or less than about 50% of antigen-specific T_{eff} cells and/or Tₑₘ cells are positive for PD-1, CTLA-4 or LAG-3. In still other particular embodiments, less than about 15%, less than about 20%, less than about 30%, less than about 40% or less than about 50% of antigen-specific T_{eff} cells and Tₑₘ cells are positive for PD-1, CTLA-4, or LAG-3. In instances where the sequential heterologous boost method is a method of inducing an immune response to at least two neoantigens of interest in a subject, such an effect may be observed with respect to the immune response induced to least one or the antigens of interest. In other instances where the sequential heterologous boost method is a method of inducing an immune response to at least two neoantigens of interest in a subject, such an effect may be observed with respect to the aggregate immune response to the antigens of interest.

The sequential heterologous boost methods described herein utilize consecutive heterologous boosts, which are consecutive boosts wherein one of the boosts comprising a first oncolytic virus and the other boost comprising a second oncolytic virus that is immunologically distinct from the first oncolytic virus. In certain embodiments, the sequential heterologous boost methods described herein comprise two boosts, a first boost that comprises a first oncolytic virus, and a second, consecutive, heterologous boost comprising a second oncolytic virus that is immunologically distinct from the first oncolytic virus. In certain embodiments, the sequential heterologous boost methods described herein comprise more than two boosts, e.g., comprise 3, 4, 5 or more boosts, wherein any consecutive pair of boosts utilizes heterologous boosts.

For example, in certain embodiments, the sequential heterologous boost methods described herein comprise three boosts wherein the oncolytic virus of the first boost is immunologically distinct from the oncolytic virus of the second boost, and the oncolytic virus of the second boost is immunologically distinct from the oncolytic virus of the third boost. Such methods may comprise two or three oncolytic viruses, wherein the oncolytic viruses are distributed in the boosts in a manner that results in heterologous boost administration.

In another non-limiting example, the sequential heterologous boost methods described herein comprise four boosts wherein the oncolytic virus of the first boost is immunologically distinct from the oncolytic virus of the second boost, the oncolytic virus of the second boost is immunologically distinct from the oncolytic virus of the third boost, and the oncolytic virus of the third boost is immunologically distinct from the oncolytic virus of the fourth boost. Such methods may comprise two, three or four oncolytic viruses, wherein the oncolytic viruses are distributed in the boosts in a manner that results in heterologous boost administration.

In yet another non-limiting example, the sequential heterologous boost methods described herein comprise five boosts wherein the oncolytic virus of the first boost is immunologically distinct from the oncolytic virus of the second boost, the oncolytic virus of the second boost is immunologically distinct from the oncolytic virus of the third boost, the oncolytic virus of the third boost is immunologically distinct from the oncolytic virus of the fourth boost, and the oncolytic virus of the fourth boost is immunologically distinct from the oncolytic virus of the fifth boost. Such methods may comprise two, three, four or five oncolytic viruses, wherein the oncolytic viruses are distributed in the boosts in a manner that results in heterologous boost administration.

In one embodiment, a sequential heterologous boost method of inducing an immune response to a neoantigen in a subject comprises: (a) administering to the subject a dose of a priming composition that is capable of inducing an immune response to the neoantigen; (b) subsequently administering to the subject a dose of a first boost, wherein the first boost comprises a first oncolytic virus, wherein the first oncolytic virus comprises a genome that comprises a transgene or a nucleic acid sequence that encodes and expresses, in the subject, a protein that is capable of inducing an immune response to the neoantigen; and (c) subsequently administering to the subject a dose of a second, heterologous boost, wherein the heterologous boost comprises a second oncolytic virus, wherein the second oncolytic virus comprises a transgene or a nucleic acid sequence that encodes and expresses, in the subject, a protein that is capable of inducing an immune response to the neoantigen, and wherein the second oncolytic virus is immunologically distinct from the first oncolytic virus, such that an immune response to the neoantigen is induced in the subject.

In another embodiment, a sequential heterologous boost method of inducing an immune response to a neoantigen in a subject comprises: (a) administering to the subject a dose of a priming composition that is capable of inducing an immune response to the neoantigen; (b) subsequently administering to the subject a dose of a first boost, wherein the first boost comprises a first oncolytic virus, wherein the first oncolytic virus comprises a genome that comprises a transgene or a nucleic acid sequence that encodes and expresses, in the subject, a protein that is capable of inducing an immune response to the neoantigen; and (c) subsequently administering to the subject a dose of a second, heterologous boost, wherein the heterologous boost comprises a second oncolytic virus, wherein the second oncolytic virus comprises a transgene or a nucleic acid sequence that encodes and expresses, in the subject, a protein that is capable of inducing an immune response to the neoantigen, and wherein the second oncolytic virus is immunologically distinct from the first oncolytic virus; and (d) subsequently administering to the subject a dose of a third boost, wherein the third boost comprises an oncolytic virus that is immunologically distinct from the oncolytic virus of the second boost and that comprises a transgene or a nucleic acid sequence that expresses, in the subject, a protein that is capable of inducing an immune response to the neoantigen, such that an immune response to the neoantigen is induced in the subject. In particular embodiments, the oncolytic virus of the third boost is the first oncolytic virus, present in the first boost. In one non-limiting example, step (d) is performed at least about 60 days after step (b). In other non-limiting example, step (d) is performed at least about 120 days after step (b).

In certain embodiments, such a sequential heterologous boost method further comprises, subsequently to (d) a step (e) administering to the subject a dose of a fourth boost, wherein the fourth boost comprises an oncolytic virus that is immunologically distinct from the oncolytic virus of the third boost and that comprises a transgene or a nucleic acid sequence that encodes and expresses, in the subject, a protein that is capable of inducing an immune response to the neoantigen. In particular embodiments, the oncolytic virus of the fourth boost is the second oncolytic virus, present in the second boost. In one non-limiting example, step (e) is performed at least about 60 days after step (c). In other non-limiting example, step (e) is performed at least about 120 days after step (c).

In certain embodiments, such a sequential heterologous boost method further comprises, subsequently to (e) step (f) administering to the subject a dose of a fifth boost, wherein the fifth boost comprises an oncolytic virus that is immunologically distinct from the oncolytic virus of the fourth boost and that comprises a transgene or a nucleic acid sequence that encodes and expresses, in the subject, a protein that is capable of inducing an immune response to the neoantigen. In particular embodiments, the oncolytic virus of the fifth boost is the first oncolytic virus, present in the first boost. In other particular embodiments, the oncolytic virus of the fifth boost is the oncolytic virus present in the third boost. In one non-limiting example, step f) is performed at least about 60 days after step (d). In other non-limiting example, step (f) is performed at least about 120 days after step (d).

In certain aspects, the sequential heterologous boost methods presented herein are methods of inducing an immune response to one or more neoantigens of interest in a subject, wherein the boosts are heterologous boosts and at least one of the boosts comprises (a) one or more proteins capable of inducing an immune response to the neoantigen, that is, comprises one or more antigenic proteins, and (b) an oncolytic virus that does not comprise a transgene or a nucleic acid sequence that encodes and expresses the one or more antigenic proteins. In certain other aspects, the sequential heterologous boost methods presented herein are methods of inducing an immune response to one or more neoantigens of interest in a subject, wherein the boosts are heterologous boosts and at least one of the boosts comprises (a) one or more proteins capable of inducing an immune response to the one neoantigen(s) of interest, that is, comprises one or more antigenic proteins, and (b) an oncolytic virus that comprises a transgene or a nucleic acid sequence that encodes and expresses, in the subject, one or more proteins capable of inducing an immune response to the one or more neoantigen(s) of interest, that is, expresses one or more antigenic proteins.

In yet other aspects, the sequential heterologous boost methods presented herein are methods of inducing an immune response to one or more neoantigens of interest in a subject, wherein the boosts are heterologous boosts and 1) at least one of the boosts comprises a) one or more proteins capable of inducing an immune response to the one or more neoantigens, that is, comprises one or more antigenic proteins, and b) an oncolytic virus that does not comprise a transgene or a nucleic acid sequence that encodes and expresses the antigenic proteins; and 2) at least one of the boosts comprises a) one or more proteins capable of inducing an immune response to the one or more neoantigens of interest, that is, comprises one or more antigenic proteins, and b) an oncolytic virus that comprises a transgene or a nucleic acid sequence that encodes and expresses, in the subject, one or more proteins capable of inducing an immune response to the one or more neoantigens of interest, that is, expresses one or more antigenic proteins.

For example, in certain embodiments, a sequential heterologous boost method of inducing an immune response to a neoantigen in a subject presented herein, comprises a) administering to the subject a dose a priming composition; b) subsequently administering to the subject a dose of a first boost, wherein the first boost comprises a protein that is capable of inducing an immune response to the neoantigen, and a first oncolytic virus that does not comprise a transgene or a nucleic acid sequence that expresses the protein, wherein the protein and the first oncolytic virus are administered to the subject together or separately; and c) subsequently administering to the subject a dose of a second, heterologous boost, wherein the heterologous boost comprises a protein that is capable of inducing an immune response to the neoantigen, and a second oncolytic virus that does not comprise a transgene or a nucleic acid sequence that encodes and expresses the protein, wherein the protein and the second oncolytic virus are administered to the subject together or separately, and wherein the second oncolytic virus is immunologically distinct from the first oncolytic virus, such that an immune response to the neoantigen is induced in the subject. In particular embodiments, such sequential heterologous boost methods may comprise additional heterologous boosts, for example a third, fourth or fifth heterologous boost.

In one embodiment of the sequential heterologous boost methods described herein, at least one of the oncolytic viruses is a rhabdovirus. In a particular embodiment, the rhabdovirus is a Farmington virus. In another particular embodiment, the rhabdovirus is a Maraba virus, e.g., is an MG1 virus. In another embodiment, the first oncolytic virus and the second oncolytic virus are rhabdoviruses. In a particular embodiment, at least one of the rhabdoviruses is a Farmington virus. In another particular embodiment, at least one of the rhabdoviruses is a Maraba virus, e.g., is an MG1 virus. In yet another embodiment, one of the rhabdoviruses is a Farmington virus and one of the rhabdoviruses is a Maraba virus, e.g., an MG1 virus. In a specific embodiment, the first oncolytic virus is a Farmington virus and the second oncolytic virus is a Maraba virus, e.g., an MG1 virus. In another specific embodiment, the first oncolytic virus is a Maraba virus, e.g., an MG1 virus, and the second oncolytic virus is a Farmington virus.

In one embodiment of the sequential heterologous boost methods described herein, at least one of the oncolytic viruses is an adenovirus, a vaccinia virus, a measles virus, or a vesicular stomatitis virus. In another embodiment, the first and the second oncolytic virus are an adenovirus, a vaccinia virus, a measles virus, or a vesicular stomatitis virus. In a particular embodiment, either the first or the second oncolytic virus is a rhabdovirus and the other oncolytic virus is a vaccinia virus. In a specific embodiment, the first oncolytic virus is a rhabdovirus and the second oncolytic virus is a vaccinia virus. In another specific embodiment, first oncolytic virus is a vaccinia virus and the second oncolytic virus is a rhabdovirus. In a non-limiting example of such sequential heterologous boost methods, the rhabdovirus is a Farmington virus. In another such non-limiting example, the rhabdovirus is a Maraba virus, e.g., an MG-1 virus. In yet another such non-limiting example, the vaccinia virus is a CopMD5p, CopMD3p, CopMD5p3p, or SKV vaccinia virus.

In another embodiment, at least one of the oncolytic viruses is a rhabdovirus and at least one of the oncolytic viruses is a vaccinia virus, e.g., a CopMD5p, CopMD3p, CopMD5p3p or SKV vaccinia virus. In another example of such sequential heterologous boost methods, the oncolytic viruses comprise at least one Farmington virus and at least one vaccinia virus, e.g., a CopMD5p, CopMD3p, CopMD5p3p, or SKV vaccinia virus. In another example, the oncolytic viruses comprise at least one Maraba virus, e.g., an MG-1 virus and at least one vaccinia virus, e.g., a CopMD5p, CopMD3p, CopMD5p3p, or SKV vaccinia virus. In yet another example the oncolytic viruses comprise at least one Farmington virus, at least one Maraba virus, e.g., an MG-1 virus, and at least one vaccinia virus, e.g., a CopMD5p, CopMD3p, CopMD5p3p, or SKV vaccinia virus.

As used herein throughout, when two or more elements, may be administered together or separately, such elements may, e.g., be administered as a single composition or as part of more than one composition, and may be administered concurrently (whether as part of a single composition or as part of more than one composition), or sequentially.

In another embodiment, a sequential heterologous boost method of inducing an immune response to a plurality of neoantigens of interest in a subject comprises (a) administering to the subject a dose of a priming composition, wherein the priming composition induces an immune response to the plurality of neoantigens; (b) subsequently administering to the subject a dose of a first boost, wherein the first boost comprises a protein composition that is capable of inducing an immune response to the plurality of neoantigens of interest, and a first oncolytic virus that does not comprise a transgene or nucleic acid sequence that expresses, in the subject, a protein composition that is capable of inducing an immune response to any of the plurality of neoantigens of interest; and (c) subsequently administering to the subject a dose of a second, heterologous boost, wherein the heterologous boost comprises a protein composition that is capable of inducing an immune response to the plurality of neoantigens of interest, and a second oncolytic virus that does not comprise a transgene or nucleic acid sequence that expresses, in the subject, a protein composition that is capable of inducing an immune response to any of the plurality of neoantigens of interest, and wherein the second oncolytic virus is immunologically distinct from the first oncolytic virus, such that an immune response to plurality of neoantigens is induced in the subject. In particular embodiments, such sequential heterologous boost methods may comprise additional heterologous boosts, for example a third, fourth or fifth heterologous boost. In certain such embodiments, the protein composition in b) that is capable of inducing an immune response to the plurality of neoantigens of interest, and protein composition in c) that is capable of inducing an immune response to the plurality of neoantigens of interest may comprise one or more antigenic proteins. In particular embodiments, the protein composition in b) and the protein composition in c) are not identical. In certain such embodiments, a plurality of antigens of interest may be 2 to about 20 antigens, e.g., 2 to about 10 antigens, 2-5 antigens, for example 2, 3, 4 or 5 antigens.

In another embodiment, a sequential heterologous boost method of inducing an immune response to a plurality of neoantigens of interest in a subject comprises a) administering to the subject a dose of a priming composition, wherein the priming composition induces an immune response to the plurality of neoantigens; b) subsequently administering to the subject a dose of a first boost, wherein the first boost comprises a first protein composition that is capable of inducing an immune response to at least one of the plurality of neoantigens of interest, and a first oncolytic virus that comprises one or more transgenes or nucleic acid sequences that express, in the subject, a second protein composition that is capable of inducing an immune response to at least one of the plurality of neoantigens of interest, such that, as a whole the first protein composition and the second protein composition are capable of inducing an immune response to the plurality of neoantigens of interest; and c) subsequently administering to the subject a dose of a second, heterologous boost, wherein the heterologous boost comprises a third protein composition that is capable of inducing an immune response to at least one of the plurality of neoantigens of interest, and a second oncolytic virus that comprises one or more transgenes or nucleic acid sequences that express, in the subject, a fourth protein composition that is capable of inducing an immune response to at least one of the plurality of neoantigens of interest such that, as a whole the first protein composition and the second protein composition are capable of inducing an immune response to the plurality of neoantigens of interest, and wherein the second oncolytic virus is immunologically distinct from the first oncolytic virus, such that an immune response to plurality of neoantigens is induced in the subject.

In particular embodiments, such sequential heterologous boost methods may comprise additional heterologous boosts, for example a third, fourth or fifth heterologous boost. In certain such embodiments, the first, second, third, and fourth protein composition may comprise one or more antigenic proteins. In particular embodiments, the first, second, third, and/or fourth protein compositions are not identical. In certain such embodiments, a plurality of antigens of interest may be 2 to about 20 antigens, e.g., 2 to about 10 antigens, 2-5 antigens, for example 2, 3, 4 or 5 antigens.

For example, in one embodiment, a sequential heterologous boost method of inducing an immune response to at least two antigens in a subject comprises a) administering to the subject a dose of a priming composition, wherein the priming composition induces an immune response to at least a first and a second neoantigen; b) subsequently administering to the subject a dose of a first boost, wherein the first boost comprises a first oncolytic virus that comprises a transgene or nucleic acid sequene that expresses, in the subject, a protein that is capable of inducing an immune response to at least the first neoantigen and a nucleic acid that expresses, in the subject, a protein that is capable of inducing an immune response to at least the second neoantigen; and c) subsequently administering to the subject a dose of a second, heterologous boost, wherein the heterologous boost comprises a second oncolytic virus that comprises a genome comprising a nucleic acid sequence that expresses, in the subject, a protein that is capable of inducing an immune response to at least the first neoantigen and a nucleic acid sequence that expresses, in the subject, a protein that is capable of inducing an immune response to at least the second neoantigen, and wherein the second oncolytic virus is immunologically distinct from the first oncolytic virus, such that an immune response to at least the first and the second neoantigens is induced in the subject. In particular embodiments, such sequential heterologous boost methods may comprise additional heterologous boosts, for example a third, fourth or fifth heterologous boost.

In certain embodiments of any of the sequential heterologous boost methods described herein, a dose of a priming composition that induces an immune response against greater than one antigen of interest may, for example, involve the administration of a single composition to a subject, or may involve the administration of more than one composition to the subject. For example, in instances where the priming composition is designed to induce an immune response to at least two neoantigens of interest, the prime dose may, in alternative embodiments, comprise a composition that comprise a composition that induces an immune response to at least the first and the second neoantigens, or, may comprise a first composition and a second composition, wherein the first composition induces an immune response to at least the first neoantigen, and the second composition induces an immune response to at least the second neoantigen. In embodiments where the prime dose comprises more than one composition, the compositions may be administered together or separately.

A dose e.g., a prime dose, a dose of a first boost, a dose of a second boost, a dose of a third boost and the like, as used herein, refers to an amount sufficient to achieve a recited or intended goal. In certain embodiments, a dose may be administered as a single composition. In other embodiments, a dose may be administered in parts. When administered in parts, e.g., 2, 3, or 4 parts, the parts may be administered concurrently or sequentially.

In certain embodiments of the sequential heterologous boost methods presented herein, the prime dose comprises a virus. In such embodiments, a prime dose may, for example, comprise about 1×10⁷ particle forming units (PFU) to about 5×10¹² PFU of virus. In certain embodiments, the prime dose comprises about 1×10¹¹ PFU, or 2×10¹¹ PFU of virus. In particular embodiments, the virus comprises a genome that comprises a transgene or a nucleic acid sequence that expresses, in a subject, antigenic protein, as described herein. In other particular embodiments, the virus is a virus that does not comprise a nucleic acid that expresses the antigenic protein, as described herein. In certain embodiments, the virus is an adenovirus, for example, a serotype 5 adenovirus, e.g., a recombinant replication-incompetent human Adenovirus serotype 5.

In certain embodiments wherein a prime dose comprises one or more proteins capable of inducing an immune response to one or more neoantigens of interest, that is, comprises one or more antigenic proteins, the dose of such a prime may comprise about 10 μg to about 1000 μg of the one or more antigenic proteins. In particular embodiments, these amounts refer to the amount of antigenic protein present in a prime dose in the aggregate. In other particular embodiments, these amounts refer to the amount of each antigenic protein present in the prime dose.

In certain embodiments of the sequential heterologous boost methods presented herein, a dose of a priming composition is administered to a subject about 7 to about 90 days immediately prior to the administration of a first boost dose to the subject. In particular embodiments, a dose of a priming composition is administered to a subject about 7 to 21 days, about 7 to 28 days, about 14 to about 60 days, about 14 to about 28 days, about 28 to about 60 days, about 14 days, about 15 days, about 21 days, about 28 days, about 29 days, about 30 days, about 50 days or about 60 days immediately prior to the administration of a first boost dose to the subject. For example, in certain embodiments of the sequential heterologous boost methods presented herein, a dose of a priming composition is administered to a subject about 7 to about 90 days immediately prior to the administration of a first boost dose to the subject. In particular embodiments, a dose of a priming composition is administered to a subject about 7 to about days, 14 to about 60 days, about 14 to about 28 days, about 28 to about 60 days, about 14 days, about 15 days, about 21 days, about 28 days, about 29 days, about 30 days, about 50 or about 60 days immediately prior to the administration of a first boost dose to the subject. In particular embodiments, a second, heterologous boost dose is administered to the subject about 2 weeks to about 3 months after the first boost dose is administered to the subject.

In particular embodiments, the first boost dose is administered to the subject about 7 to 21 days, about 7 to 28 days, about 14 to about 60 days, about 14 to about 28 days, about 28 to about 60 days, about 14 days, about 15 days, about 21 days, about 28 days, about 29 days, about 30 days, about 50 days or about 60 days after the dose of the priming composition is administered to the subject. In particular embodiments, the first boost dose is administered to the subject about 2 weeks to about 4 weeks, about 2 weeks to about 8 weeks, about 2 weeks to about 12 weeks, about 2 weeks, about 3 weeks, or about 4 weeks after the dose of the priming composition is administered to the subject. In particular embodiments, the first boost dose is administered to the subject about 2 weeks to about 3 months after the dose of the priming composition is administered to the subject.

In certain embodiments, a prime dose may be administered as a single composition. In other embodiments, a prime dose may be administered in parts. When a prime dose is administered in parts, e.g., 2, 3, or 4 parts, the parts may be administered concurrently or sequentially. Administration of a prime dose is complete prior to the initiation of the administration of the first boost dose.

In certain embodiments, administration of prime dose is performed intravenously, intramuscularly, intraperitonealy, or subcutaneously. In a particular embodiment, administration of a prime does is performed intravenously. In instances where a prime dose is administered in parts, the parts may be administered by the same or different routes of administration.

In certain embodiments of the sequential heterologous boost methods presented herein, the dose of one or more of the boosts comprises about 1×10⁷ particle forming units (PFU) to about 5×10¹² PFU of oncolytic virus. In certain embodiments, the dose of the first boost comprises an about 10-fold to an about 100-fold higher amount of oncolytic virus than the dose of the subsequent boost(s). In particular embodiments, the oncolytic virus comprises a nucleic acid that expresses, in a subject, antigenic protein, as described herein. In other particular embodiments, the oncolytic virus is an oncolytic virus that does not comprise a nucleic acid that expresses the antigenic protein, as described herein.

In certain embodiments wherein a boost dose comprises one or more proteins capable of inducing an immune response to one or more neoantigens of interest, that is, comprises one or more antigenic proteins, the dose of such a boost dose may comprise about 10 μg to about 1000 μg of the one or more antigenic proteins. In particular embodiments, these amounts refer to the amount of antigenic protein present in a boost dose in the aggregate. In other particular embodiments, these amounts refer to the amount of each antigenic protein present in the boost dose.

In certain embodiments, one or more boost doses may be administered as a single composition. In other embodiments, each of the boost doses may be administered as a single composition. In certain embodiments, any of the boost doses may be administered in parts. In other embodiments, each of the boost doses may be administered in parts. In still other embodiments, a first boost dose may be administered in parts, and subsequent boost doses are administered as a single composition. When a boost dose is administered in parts, e.g., 2, 3, or 4 parts, the parts may be administered concurrently or sequentially. Administration of a boost dose is complete prior to the initiation of the administration of the next consecutive boost, if any.

In instances where a prime dose is administered in parts, the timing of the administration of the first dose may be measured from the administration of any of the parts of the prime dose. For example, in instances where the prime dose is administered in parts and the parts are administered sequentially, the timing of the administration of the first boost dose may be measured from the administration of the first part of the prime dose or, e.g., from the administration of the final part of the prime dose. In instances where a first boost dose is administered in parts, generally the timing of administration of the first boost dose is measured from the initiation of the first boost, that is, from the administration of the first part of the boost dose.

In certain embodiments of the sequential heterologous boost methods presented herein, a boost dose is administered to a subject about 7 to about 90 days after the immediately prior boost dose is administered to a subject. In particular embodiments, a boost dose is administered to the subject about 7 to 21 days, about 7 to 28 days, about 14 to about 60 days, about 14 to about 28 days, about 28 to about 60 days, about 14 days, about 15 days, about 21 days, about 28 days, about 29 days, about 30 days, about 50 days or about 60 days after an immediately prior dose is administered to the subject. For example, in certain embodiments of the sequential heterologous boost methods presented herein, a second, heterologous boost dose is administered to a subject about 7 to about 90 days after the first boost dose is administered to a subject. In particular embodiments, a second, heterologous boost dose is administered to the subject about 7 to about days, 14 to about 60 days, about 14 to about 28 days, about 28 to about 60 days, about 14 days, about 15 days, about 21 days, about 28 days, about 29 days, about 30 days, about 50 or about 60 days after the first boost dose is administered to the subject. In particular embodiments, a second, heterologous boost dose is administered to the subject about 2 weeks to about 3 months after the first boost dose is administered to the subject.

In other particular embodiments, boosts are administered using a cycle that leaves about 28 days, 30 days, or 60 days between boosts. In one such embodiment, the cycle alternates use of a boost comprising a first oncolytic virus followed by a second oncolytic virus and leaves about 28 days, 30 days, or 60 days between boosts. In one example of such a cycle, one boost comprises a Farmington virus and the other boost comprises a Maraba virus, e.g., an MG1 virus. In another example of such a cycle, one boost comprises a Farmington virus and the other boost comprises a vaccinia virus, e.g., a CopMD5p, CopMD3p, CopMD5p3p or SKV vaccinia virus. In yet another example of such a cycle, one boost comprises a Maraba virus, e.g., an MG1 virus, and the other boost comprises a vaccinia virus, e.g., a CopMD5p, CopMD3p, CopMD5p3p or SKV vaccinia virus.

In certain embodiments of the sequential heterologous boost methods presented herein, a boost dose is administered to a subject about 2 weeks to about 8 weeks after the immediately prior boost dose is administered to a subject. In particular embodiments, a boost dose is administered to the subject about 2 weeks to about 4 weeks, about 2 weeks to about 8 weeks, about 2 weeks to about 12 weeks, about 2 weeks, about 3 weeks, or about 4 weeks after the immediately prior boost dose is administered to the subject. For example, in certain embodiments of the sequential heterologous boost methods presented herein, a second, heterologous boost dose is administered to a subject about 2 weeks to about 8 weeks after the first boost dose is administered to a subject. In particular embodiments, a second, heterologous boost dose is administered to the subject about 2 weeks to about 4 weeks, about 2 weeks to about 8 weeks, about 2 weeks to about 12 weeks, about 2 weeks, about 3 weeks, or about 4 weeks after the first boost dose is administered to the subject.

In instances where an immediately prior boost is administered in parts, the timing of the administration of the immediately prior boost dose may be measured from the administration of any of the parts of the immediately prior boost dose. For example, in instances where the immediately prior boost dose is administered in parts and the parts are administered sequentially, the timing of the administration of the immediately prior boost dose may be measured from the administration of the first part of the immediately prior dose or, e.g., from the administration of the final part of the immediately prior dose. In instances involving the timing between two consecutive boosts wherein at least the later of the two consecutive boosts is administered in parts, generally the timing of the administration of the later of the two consecutive boost doses is measured from the initiation of the later boost, that is, from the administration of the first part of the later boost dose.

In certain embodiments, administration of at least one boost dose is performed intravenously, intramuscularly, intraperitonealy, or subcutaneously. In a particular embodiment, at least one boost dose is performed intravenously. In particular embodiments, each of the boost doses is performed intravenously. In instances where a boost dose is administered in parts, the parts may be administered by the same or different routes of administration.

In a specific embodiment, the methods of inducing an immune response to one or more neoantigens described herein treat the subject's cancer. In some embodiments, a method of inducing an immune response to one or more neoantigens described herein results in one, two, three or more of the following effects: complete response, partial response, objective response, increase in overall survival, increase in disease free survival, increase in objective response rate, increase in time to progression, stable disease, increase in progression-free survival, increase in time-to-treatment failure, and improvement or elimination of one or more symptoms of cancer. In a specific embodiment, a method of inducing an immune response to one or more neoantigens described herein results in an increase in overall survival of the subject. In another specific embodiment, a method of inducing an immune response to one or more neoantigens described herein results in an increase in progression-free survival of the subject. In another specific embodiment, a method of inducing an immune response to one or more neoantigens described herein results an increase in overall survival of the subject and an increase in progression-free survival. In a specific embodiment, the methods of inducing an immune response to one or more neoantigens described herein may result in a decrease in tumor burden from baseline (e.g., 10%, 15%, 20%, 25%, 30%, 35%, 40%, 45%, 50%, 55% or more, or 10% to 25%, 25% to 50%, or 25% to 75% decrease in tumor burden from baseline).

In specific embodiments, a subject treated in accordance with the methods described herein has metastatic cancer. In another specific embodiment, a subject treated in accordance with the methods described herein has unresectable cancer. In another specific embodiment, a subject treated in accordance with the methods described herein has metastatic, unresectable cancer. In another specific embodiment, a subject treated in accordance with the methods described herein has recurrent cancer. The recurrent cancer may be metastatic and unresectable.

Cancers that may be treated in accordance with the methods described herein include colorectal cancer, breast cancer, ovarian cancer, cervical cancer, uterine cancer, salivary gland cancer, liver cancer bone cancer, brain cancer, pancreatic cancer, thyroid cancer, skin cancer, and lung cancer. Specific examples of cancers that may be treated in accordance with the methods described herein include renal cell carcinoma, melanoma, squamous cell carcinoma, mesothelioma, non-Hodgkin's disease, sarcoma, Hodgkin's disease, endometrial carcinoma, esophageal cancer, glioblastoma multiforme, hepatocellular carcinoma, or head and neck squamous cell carcinoma. The cancer may be advanced (e.g., locally advanced), metastatic, unresectable, recurrent, or a combination of any of the foregoing. In some embodiments, the cancer is refractory or resistant to standard therapy (e.g., chemotherapy).

### 5.6 Kits

In one aspect, provided herein is a pharmaceutical pack or kit comprising one or more components necessary to practice a method described herein. In one embodiment, provided herein is a pharmaceutical pack or kit comprising a priming composition(s) and a composition(s) for a first boost, wherein the compositions or the components of each of the compositions may be in a separate container. In one embodiment, provided herein is a pharmaceutical pack or kit comprising a composition(s) for first boost composition and a composition(s) for a second boost, wherein the compositions or the components of each composition for each boost may be in a separate container. In another embodiment, provided herein is a pharmaceutical pack or kit comprising compositions for two or more boosts described herein, wherein the compositions or the components of each composition for each boost may be in a separate container. In another embodiment, provided herein is a pharmaceutical pack or kit comprising a priming composition and compositions for two or more boosts described herein, wherein the compositions or the components of each composition for each boost and priming composition may be in a separate container. In a specific embodiment, the pack or kit further comprises instructions for each of the compositions in the heterologous boost method described herein. In some embodiments, the pack or kit further one or more components: (1) to determine if the subject has pre-existing immunity to a neoantigen, (2) to assess the immune response induced following one or steps of a heterologous boost method described herein, or (3) both (1) and (2).

### 6. EXAMPLES

### 6.1 Example 1

### 6.1.1 Materials and Methods

Mouse Models. All animal procedures were performed in accordance with the institutional guidelines of the University of Ottawa committee on the Use of Live Animals in Teaching and Research in accordance with guidelines established by the Canadian Council on Animal Care.

Six- to eight-week old C57BL/6 female mice were purchased from Charles River Canada (Constant, QC, Canada) and allowed to acclimatize for at least one week prior to the study start date. No special diet was used for any study. Mice were kept in sterile isolation cages and maintained on a 12-hr dark-light cycle.

Naïve Mice. 7-10 weeks old female C57BL/6 mice were primed at day 0 with either one or more peptides at 50 μg subcutaneously (SC) with adjuvant: 30 μg of anti-CD40 antibody (BioXCell) and 10 μg of poly I:C (manufacturer unknown) or AVT01 M05 MC38 (4 nmol), or AVT01 M05 B16 (4 nmol)) or AVT01 M10 MC38 and B16 (2 nmol) or AVT01 individual neoantigens (8 nmol). Mice were boosted intravenously with 3×10⁸ PFU FMT or MG1 virus expressing M5 MC-38-derived (Adpgk, Reps 1, Irgq, Cpne1, Aatf) plus M5 B16.F10-derived (Obs11, Snx5, Pbk, Atp11a, Eef2) neoantigens, as listed in Table 1, in a conventional random order (FMT-N10 or MG1-N10). Alternatively mice were boosted intravenously with MG1 plus N10 peptides (50 μg each peptide subcutaneously (SC). Non-terminal peripheral blood samples were collected at specific days following the first boost and the second boost and in some cases at later time points for quantification of antigen-specific T cells by ex vivo peptide stimulation and intracellular cytokine staining (ICS) assay.

Rhabdovirus Titration. Rhabdoviruses were titred on Vero cells seeded into 6-well plates (5×10⁵ cells per well). The next day 100 μl of serial viral dilutions were prepared and added for 1 hour to Vero cells. After viral adsorption, 2 ml of agarose overlay was added (1:1, 1% agarose:2× Dulbecco's modified Eagle's medium and 20% FCS). Plaques were counted the following day. Where applicable, diameters were measured and plaque area calculated using the following formula Area=πr².

Rhabdovirus Booster Vaccines. Rhabdoviruses were diluted in order to deliver 3×10⁸ PFU per mouse in 100 μL DPBS. Mice were placed in a restrainer, and the tail was immersed in warm water or under a heat lamp until the vein is visible. 70% ethanol was used to swab the tail, and mice were then injected with 100 μL of virus (corresponding to a dose of 3×10⁸ PFU) IV via the tail vein.

For experiments involving MG1 nr boosts in the presence of loose peptides, loose peptides were administered at 50 μg per peptide in 100-200 μL IV (mixed with virus).

Flow Cytometry Antibodies. The following antibodies used for flow cytometry were purchased from BD Biosciences: anti-CD8α (clone 53-6.7); anti-IFN-γ (clone XMG1.2); anti-TNF-α (clone MP6-XT22); anti-IL-2 (clone JES6-5H4). Fixable viability dye (eFluor 780 or eFluor 450) was purchased from eBioscience. Results from stained samples were acquired using a LSR (BD Biosciences) and analyzed using FlowJo (Tree Star, Ashland, Oreg.).

Preparation of Tissues for Flow Cytometry. Non-terminal peripheral blood samples were collected from the saphenous vein into heparinized tubes (Microvette CB 300; SARSTEAD AG&Co). Blood was stored overnight at 4° C. prior to processing or processed immediately. Red blood cells were removed by treatment with a 0.15 mol/l NH₄Cl lysis buffer (pH 7.4). The isolated peripheral blood mononuclear cells (PBMCs) were resuspended in RPMI-10 medium and used for further downstream experiments.

Intracellular Cytokine Staining (ICS). PBMCs suspended in complete RPMI were added to round-bottom 96-well plates and restimulated with 5 μg/ml of peptide (one of five MC-38 peptides: Adpgk (ASMTNMELM (SEQ ID NO:11)), Reps1 (AQLANDVVL (SEQ ID NO:12)), Irgq (AALLNSAVL (SEQ ID NO:13)), Cpne1 (SSPYSLHYL (SEQ ID NO:14)), Aaltf (MAPIDHTTM (SEQ ID NO:15)); or one of five B16.F10 peptides: Obs11 (LCPGNKYEM (SEQ ID NO:16)), Snx5 (R373Q) (AAFQKNLIEM (SEQ ID NO:17)), Pbk (AAVILRDAL (SEQ ID NO:18)), Atp11a (QSLGFTYL (SEQ ID NO:19)) and Eef2 (VKAYLPVNESFAFTA (SEQ ID NO:20)); 1 μg/ml Maraba N₅₂₋₅₉ peptide (RGYVYQGL (SEQ ID NO:21)); C57BL/6 mice); or FMT N₃₀₁₋₃₀₉ (AVVLMFAQC (SEQ ID NO:22)) for 4 hours at 37° C. Negative (unstimulated) controls received DMSO in RPMI. Positive control wells received PMA (100 ng/ml) plus ionomycin (1 μg/ml). After 1 hour, Brefeldin A (0.2 μl/well; BD Biosciences) was added to each well. After stimulation, cells were washed with normal RPMI medium containing 10% FCS and resuspended back in this medium and stored overnight at 4° C. The next day, cells were washed twice with 0.5% BSA in PBS (FACS buffer) and incubated at 4° C. for 15 minutes with Fc block (Clone 2.4G2; BD Biosciences) diluted in FACS buffer. Cells were stained with live/dead cell marker and surface markers for 30 minutes at 4° C., then permeabilized with Cytofix/Cytoperm (BD Biosciences) according to the manufacturer's instructions. Anti-IFN-γ, anti-TNF-α, and anti-IL-2 were incubated with the samples for 30 minutes at 4° C. and cells were then washed in Perm/Wash buffer (BD Biosciences). Samples were resuspended in FACS buffer for analysis. Results are presented as numbers of cytokine-positive cells per total CD8+ T cells following peptide stimulation minus the same values obtained in control (unstimulated) samples. Results are presented as numbers of cytokine-positive cells per total CD8+ T cells following peptide stimulation minus the same values obtained in control (unstimulated) samples.

Data were acquired on BD LSR Fortessa X20 flow cytometer with HTS unit (BD Biosciences) and data were analyzed using FlowJo (TriStar) software. The debris and doublets were excluded by gating on FSC vs SSC and FSC-A vs FSC-H, respectively. Viable cells were gated based on viability dye stain. Next, CD8-positive (or CD8- and TCR-positive) cells were gated and within this population the expression of IFNγ, TNFα and IL-2 was examined. Cell numbers were calculated with the following formula:

$$N [ cell number / ml ] = \frac{N s - N u}{( \frac{V m}{W} ) * V f} * 1000$$

where N—resulting positive cell number per 1 ml of blood, Ns—number of positive cells in the well containing peptide, Nu—number of positive cells in unstimulated control, Vm—total blood volume collected from animal, W—number of wells the blood sample was distributed into, Vf—fraction of sample volume used for data acquisition by flow cytometry i.e., 80 μl out of 130 μl.

Synthesis and formulation of AVT01 compositions. Peptide-based neoantigens were produced as peptide antigen conjugates having the formula C-B1-A-B2-L-H (FIG. 15), wherein C is a charged molecule (sometimes referred to as a “charged moiety” or “charge modifying group”) consisting of multiple lysine residues that are positively charged at physiologic pH; B1 and B2 are N- and C-terminal extensions consisting of cathepsin degradable peptides, i.e. Val-Arg and Ser-Pro-Val-Cit, respectively; A is an antigenic protein described in this Section 6; L is a linker, Lys(N3-DBCO), consisting of azido-lysine (Lys(N3), CAS #159610-92-1) linked to a dibenzylcyclooctyne (DBCO; CAS #: 1353016-70-2) through a triazole bond; and, H is a hydrophobic block (sometimes referred to as a “hydrophobic molecule”) consisting of an oligopeptide, Glu(2B)-Trp-Glu(2B)-Trp-Glu(2B)-NH2, wherein each Glutamic acid residue (Glu) is linked to an imidazoquinoline-based Toll-like receptor-7 and -8 agonist (TLR-7/8a).

10 peptide-based neoantigens derived from murine tumor cell lines were prepared as peptide antigen conjugates of the formula C-B1-A-B2-L-H using the methods described in International Patent Application No. PCT/US2018/026145 (published as International Patent Application Publication No. WO 2018/187515) and U.S. Patent Application Publication No. 2020/0054741. Briefly, the 10 peptide-based neoantigens were produced as peptide antigen fragments of formula C-B1-A-B2-X1, wherein X1 is a linker precursor consisting of Lys(N3), by GenScript (Piscataway, N.J.) using standard solid-phase peptide synthesis.

Separately, a hydrophobic block (H) consisting of the oligopeptide Glu(2B)-Trp-Glu(2B)-Trp-Glu(2B)-NH2 bearing a DBCO linker precursor X2 at the N-terminus was synthesized as described in PCT/US2018/026145 (published as International Patent Application Publication No. WO 2018/187515) and U.S. Patent Application Publication No. 2020/0054741 to obtain spectroscopically pure (>95% AUC at 254 nm) white powder. MS (ESI) calculated for C₁₁₀H₁₂₆N₂₄O₁₀ m/z 1943.01, found 973.0 (M/2)⁺. Each of the peptide antigen fragments and hydrophobic block (i.e. DBCO-Glu(2B)-Trp-Glu(2B)-Trp-Glu(2B)-NH2, abbreviated 2B₃W₂) were dissolved in DMSO to a final concentration of 40 mg/mL. Each of the peptide antigen fragments were then reacted with hydrophobic block in a 1 to 1.10 mole ratio in DMSO solution at room temperature over 16 hours to yield a product solution. The product solution was evaluated by liquid chromatography in tandem with a mass spectrometer (LC-MS) to monitor reaction progress and confirm that the peptide antigen fragment was full converted to a peptide antigen conjugate by the reaction of the linker precursor X1 with the linker precursor X2 to form a covalent triazole bond. Each of the product solutions comprising a single peptide antigen conjugate were then lyophilized, re-suspended in DMSO to a final concentration of 10 mMolar and then sterile-filtered using a 0.2 μm PTFE filter membrane to yield sterile product solutions, each comprising a single peptide antigen conjugate (Table 2).

TABLE 2
Compositions of peptide antigen conjugates.

| | Peptide Antigen Conjugate | MW |
|---|---|---|
| # | Composition C-B1-A-B2-L-H | (g/mol) |
| 1 | KKKKKKKKK-VR-GIPVHLELASMTNM | 7056.19 |
| | ELMSSIVHQQVFPT-SPVZ-L-H | |
| | (SEQ ID NO: 1) | |
| 2 | KKKKKK-VR-GRVLELFRAAQLANDWL | 6648.67 |
| | QIMELCGATR-SPVZ-L-H | |
| | (SEQ ID NO: 2) | |
| 3 | KKKKKKK-VR-KARDETAALLNSAVLG | 6297.15 |
| | AAPLFVPPAD-SPVZ-L-H | |
| | (SEQ ID NO: 3) | |
| 4 | KKKKKKKKK-VR-DFTGSNGDPSSPYS | 6750.74 |
| | LHYLSPTGVNEY-SPVZ-L-H | |
| | (SEQ ID NO: 4) | |
| 5 | KKKKKKK-VR-SKLLSFMAPIDHTTMS | 6659.56 |
| | DDARTELFRS-SPVZ-L-H | |
| | (SEQ ID NO: 5) | |
| 6 | KKKKKK-VR-REGVELCPGNKYEMRRH | 6696.54 |
| | GTTHSLVIHD-SPVZ-L-H | |
| | (SEQ ID NO: 6) | |
| 7 | KKKK-VR-ELINFKRKRVAAFQKNLIE | 5825.69 |
| | MSELEIKH-SPVZ-L-H | |
| | (SEQ ID NO: 7) | |
| 8 | KKKKK-VR-DSGSPFPAAVILRDALHM | 6261.15 |
| | ARGLKYLHQ-SPVZ-L-H | |
| | (SEQ ID NO: 8) | |
| 9 | KKKKKKKK-VR-SSPDEVALVEGVQSL | 6063.85 |
| | GFTYLRLKDNYM-SPVZ-L-H | |
| | (SEQ ID NO: 9) | |
| 10 | KKKKKK-VR-FWKAYLPVNESFAFTAD | 6464.72 |
| | LRSNTGGQA-SPVZ-L-H | |
| | (SEQ ID NO: 10) | |

Note:
each functional section of the peptide antigen conjugate is separated by hyphens and the amino acid sequence comprising C-B1-A-B2 is provided as the single letter abbreviation (e.g., K = lysine), wherein Z is a non-natural amino acid Citrulline. The linker (L) and hydrophobic block (H), i.e. L-H, consist of Lys(N3-DBCO)-2B₃W₂.

To prepare the formulations referred to as AVT01 MC38 M05 (sometimes referred to as AVT01 M05 MC38 or just M05), AVT01 B16 M05 (sometimes referred to as AVT01 M05 B16 or just M05) and AVT01 M10 (or “AVT10-M10”), the sterile product solutions comprising peptide antigen conjugates 1 through 5, 6 through 10, and 1 through 10, respectively, were mixed together in an equimolar ratio to generate peptide antigen conjugate mixtures at either 2 or 1 mMolar per peptide antigen conjugate for the 5 and 10 peptide antigen conjugate mixtures, respectively. The peptide antigen conjugate mixtures were then diluted with aqueous buffer (i.e., PBS, pH 7.4) to induce spontaneous self-assembly of the peptide antigen conjugates to form mosaic nanoparticles. The aqueous solution of the peptide antigen conjugates, referred to as AVT01 MC38 M05 (peptide antigen conjugates 1-5), AVT01 B16 M05 (peptide antigen conjugates 6-10) and AVT01 M10 (peptide antigen conjugates 1-10) were then administered as prime and/or boost vaccines within 24 hours after preparation.

Statistics. For plaque size determinations, one-way analysis of variance was performed using the Bonferroni multiple comparison's test to derive a P value. For Kaplan-Meier plots, we compared survival plots using Mantel-Cox log-rank analysis. Titers and viability were compared using a two-tailed unpaired Student's T test to derive a P value. All comparisons were performed using either Graphpad Prism (Graphpad Software, La Jolla, Calif.) or Microsoft Excel.

### 6.1.2 Results

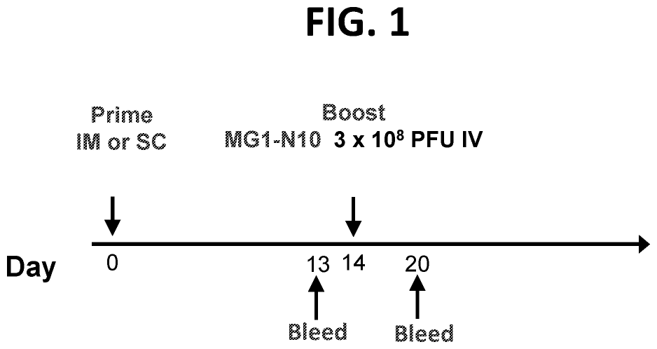

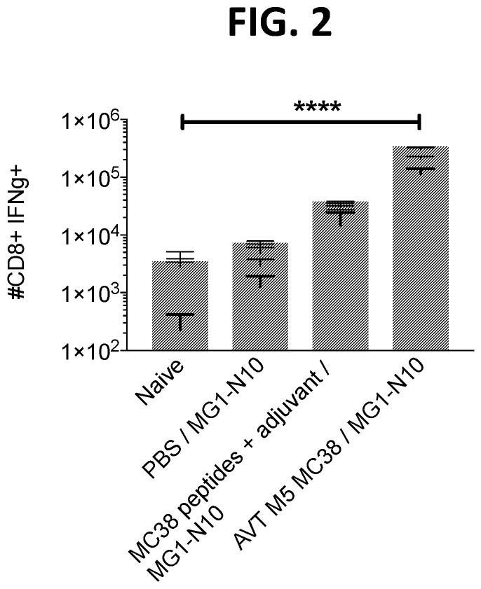

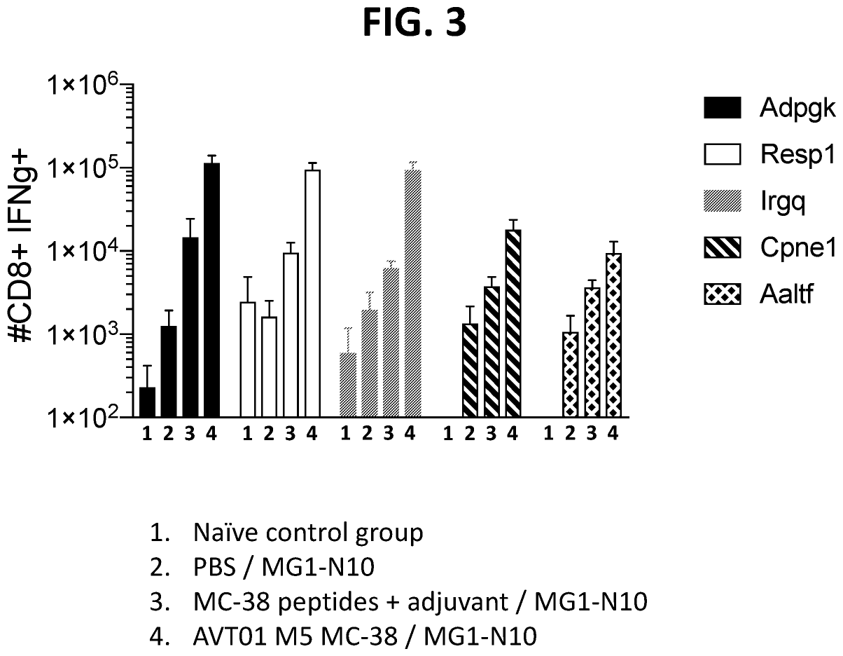

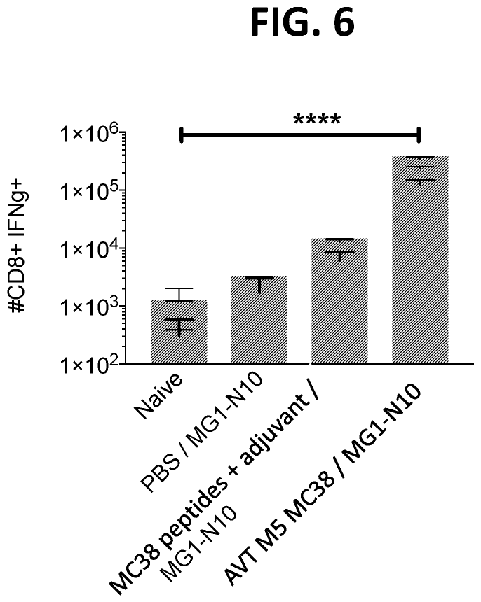

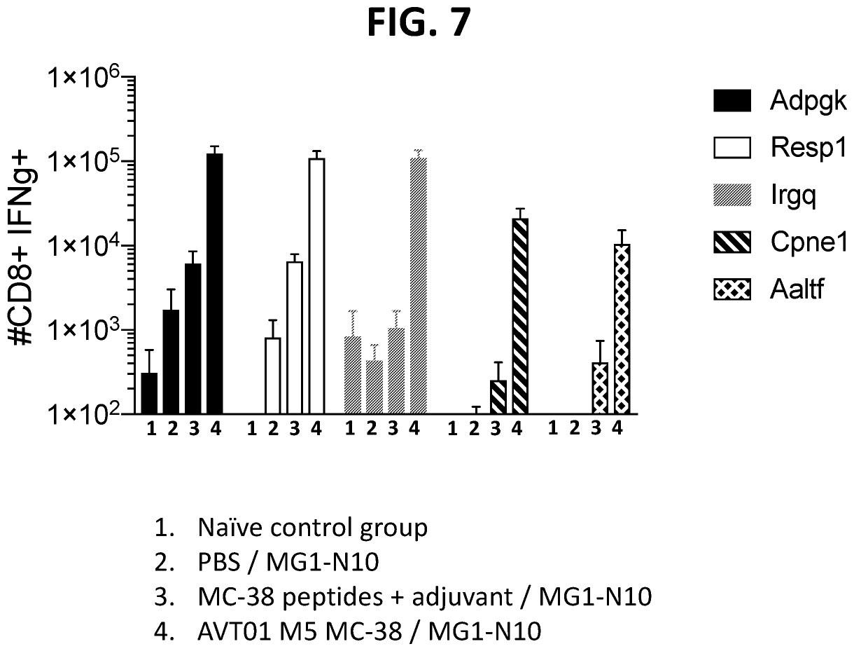

The priming of mice with AVT01 MC38 M05 or AVT01 B16 M05 yielded a greater percentage of antigen-specific CD8+ T cells than adjuvanted loose MC38 or B16 peptides after a boost with MG1-N10. The design of the experiments referred to in FIGS. 2-9 may be found in FIG. 1. FIG. 2 shows that 7 days post boost the sum of numbers of pooled MC38 antigen-specific CD8+ T cells is higher in mice primed with 4 nmol (2 nmol per injection site (IM)) of AVT01 MC38 M05 and boosted with 3×10⁸ PFU of MG1-N10 intravenously (IV) than in mice primed with Adj+MC38 loose peptides (50 μg of each peptide administered subcutaneously (SC)) and boosted with 3×10⁸ PFU of MG1-N10 intravenously (IV). FIG. 3 shows the results of that experiment with respect to individual numbers of MC 38 neoantigens (Adpgk, Reps1, Irgq, Cpne1, Aatf) specific CD8+ T cells 6 days post boost and each specific antigen shows a similar trend. FIG. 6 shows that 30 days post boost the sum of numbers of pooled MC38 antigen-specific CD8+ T cells is higher in mice primed with 4 nmol (2 nmol per injection site (IM)) of AVT01 MC38 M05 and boosted with 3×10⁸ PFU of MG1-N10 intravenously (IV) than in mice primed with Adj+MC38 loose peptides (50 μg of each peptide administered subcutaneously (SC)) and boosted with 3×10⁸ PFU of MG1-N10 intravenously (IV). FIG. 7 shows the individual numbers of MC38 antigen-specific CD8+ T cells 30 days post boost and each specific antigen shows a similar trend.

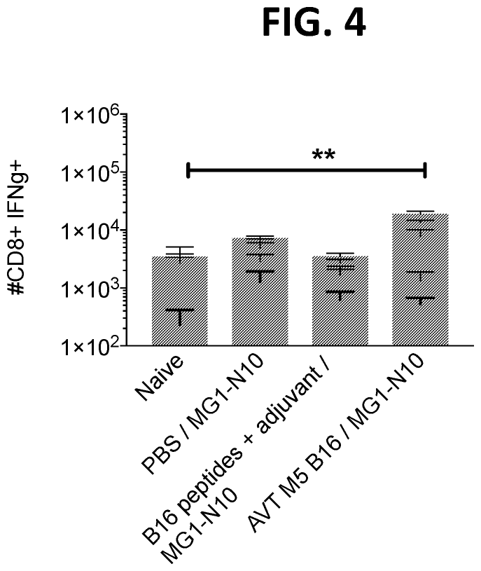

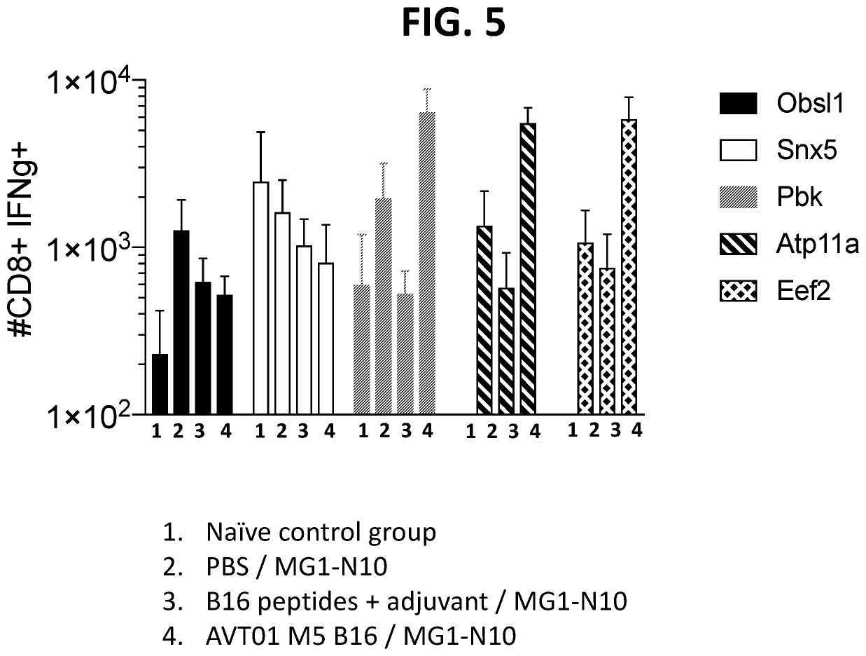

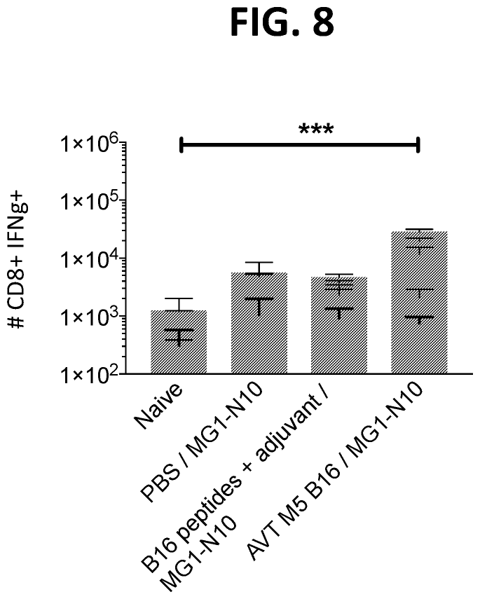

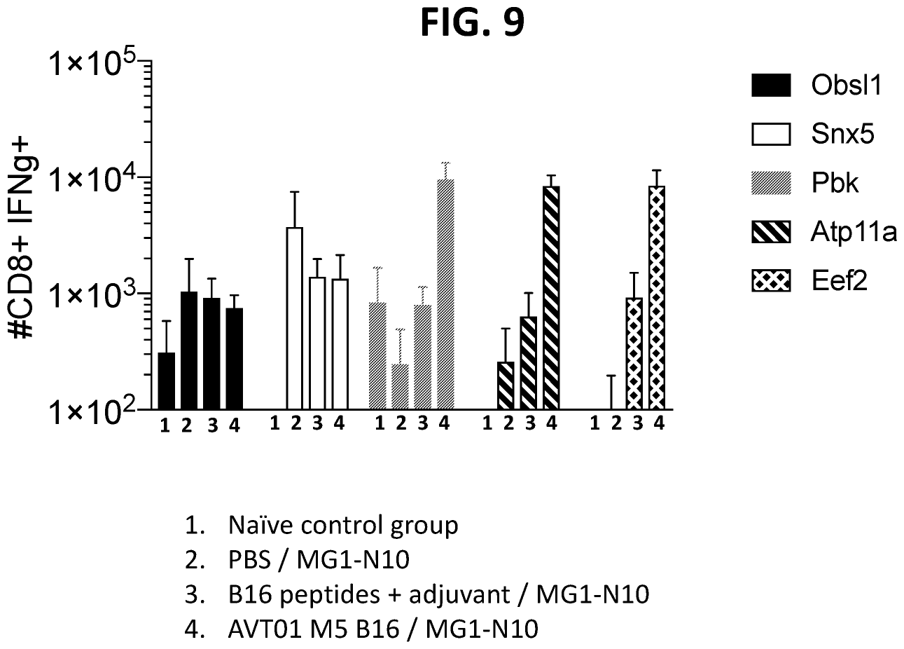

FIG. 4 shows that 6 days post boost the sum of numbers of pooled B16 antigen-specific CD8+ T cells is higher in mice primed with 4 nmol (2 nmol per injection site (IM)) of AVT01 B16 M05 and boosted with 3×10⁸ PFU of MG1-N10 intravenously (IV) than in mice primed with Adj+B16 loose peptides (50 μg of each peptide administered subcutaneously (SC)) and boosted with 3×10⁸ PFU of MG1-N10 intravenously (IV). FIG. 5 shows the results of that experiment with respect to individual numbers of B16 neoantigens (Obs11, Snx5, Pbk, Atp11a, Eef2) 6 days post boost and each specific antigen shows a similar trend. FIG. 8 shows that 30 days post boost the sum of numbers of pooled B16 antigen-specific CD8+ T cells is higher in mice primed with 4 nmol (2 nmol per injection site (IM)) of AVT01 B16 M05 and boosted with 3×10⁸ PFU of MG1-N10 intravenously (IV) than in mice primed with Adj+B16 loose peptides (50 μg of each peptide administered subcutaneously (SC)) and boosted with 3×10⁸ PFU of MG1-N10 intravenously (IV). FIG. 9 shows the individual numbers of B16 antigen-specific CD8+ T cells 30 days post boost and each specific antigen shows a similar trend.

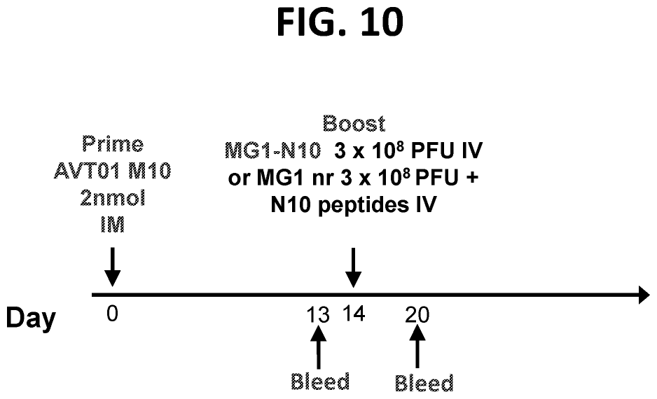

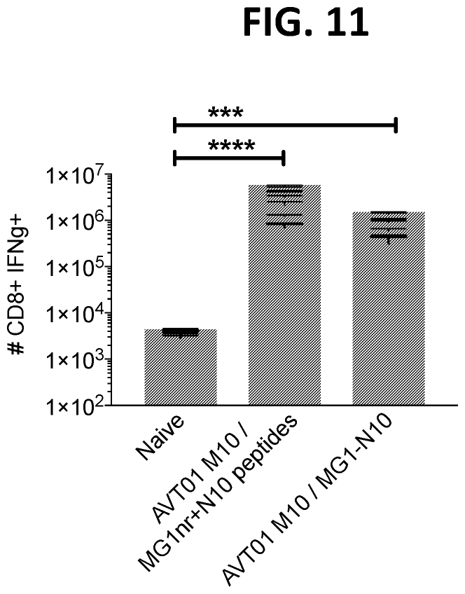

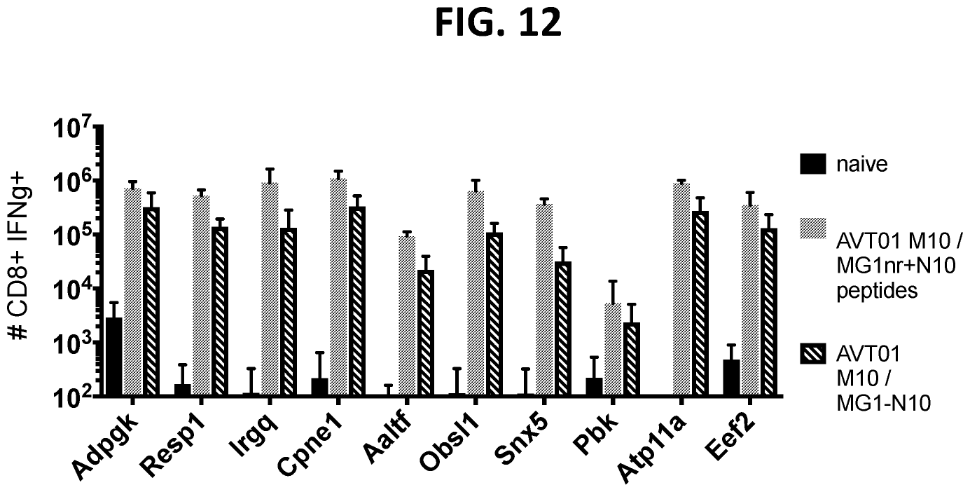

The priming of mice with AVT01 M10 and boosting with MG1 plus N10 peptides or priming of mice with AVT01 M10 and boosting with MG1-N10 resulted in expansion of antigen specific CD8+ T cells compared to the naïve control group. The design of the experiments referred to in FIGS. 11-12 may be found in FIG. 10.

Three out of thirty mice showed toxicity when injected with MG1 virus. This might be due to the lower body weights of those mice during the time of infection.

The data in FIGS. 11-12 indicate that AVT01 M10 works well as a prime. FIG. 11 shows the sum of numbers of pooled CD8+ T cells in peripheral blood expressing IFNγ in response to ex-vivo stimulation with individual antigens from mice primed with AVT01-M10 (2 nmol (1 nmol per injection site (IM)) and boosted with 3×10⁸ PFU of MG1nr intravenously (IV) plus N10 (50 μg of each peptide per mouse), mice primed with AVT01-M10 (2 nmol (1 nmol per injection site (IM)) and boosted with 3×10⁸ PFU of MG1-N10 intravenously (IV); and naïve control group (received PBS as prime and boost). FIG. 12 shows the numbers of CD8+ T cells specific to individual neoantigens 6 days post boost and each specific antigen shows a similar trend.

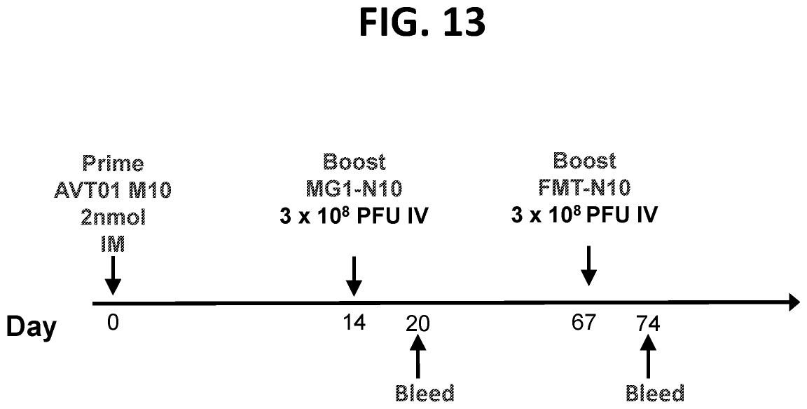

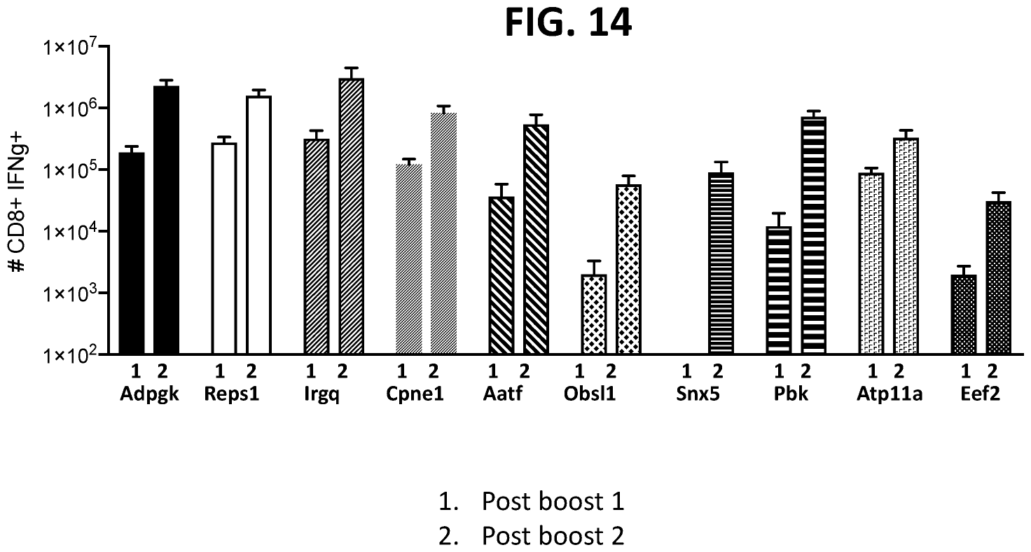

FIG. 14 shows that superboost with FMT-N10 increases the depth of immune response to MG1-N10 vaccinated mice. The design of the experiments referred to in FIG. 14 may be found in FIG. 13 Each treatment group received a prime of 8 nmol of AVT01 individual neo-antigen (one of the N10 antigens, each group received different antigen) administered IM to mice at day 0. All mice received a first boost with PBS or 3×10⁸ PFU of MG1-N10 administered IV at day 14, and a second boost of 3×10⁸ PFU of FMT-N10 administered IV at day 67. The FMT-N10 and MG1-N10 viruses were engineered to express a total of ten neoantigens (a combination of MC38 and B16 peptides). Blood samples were taken at day 20 (six days post boost 1) and day 74 (seven days post boost 2). FIG. 14 shows numbers of CD8+ T cells expressing IFNγ in response to ex-vivo stimulation with minimal epitopes corresponding to antigens used for prime in peripheral blood after boost 1 (bars labeled “1”) and after boost 2 (bars labeled “2”).

### 6.2 Example 2

### 6.2.1 Materials and Methods

Virus Booster Vaccines. Viruses were diluted in order to deliver 1×10⁸ PFU per mouse in 100 μL DPBS. Mice were placed in a restrainer, and the tail was immersed in warm water or under a heat lamp until the vein is visible. 70% ethanol was used to swab the tail, and mice were then injected with 100 μL of virus (corresponding to a dose of 1×10⁸ PFU) IV via the tail vein.

Vaccinia virus Titration. Vaccinia viruses were titred on U2OS cells seeded into 6-well plates (5×10⁵ cells per well). The next day 200 μl of serial viral dilutions were prepared and added for 2 hours to U2OS cells. After viral adsorption, 2 ml of carboxymethyl cellulose overlay was added (1:1, 3% carboxymethyl cellulose:2× Dulbecco's modified Eagle's medium and 20% FCS). Plaques were counted the following day.

Other methods, including Mouse Models, Naïve Mice, Rhabdovirus Titration, Flow Cytometry Antibodies, Preparation of Tissues for Flow Cytometry, Intracellular Cytokine Staining (ICS), Synthesis and formulation of AVT01 compositions, mirror those outlined in EXAMPLE 1.

### 6.2.2 Results

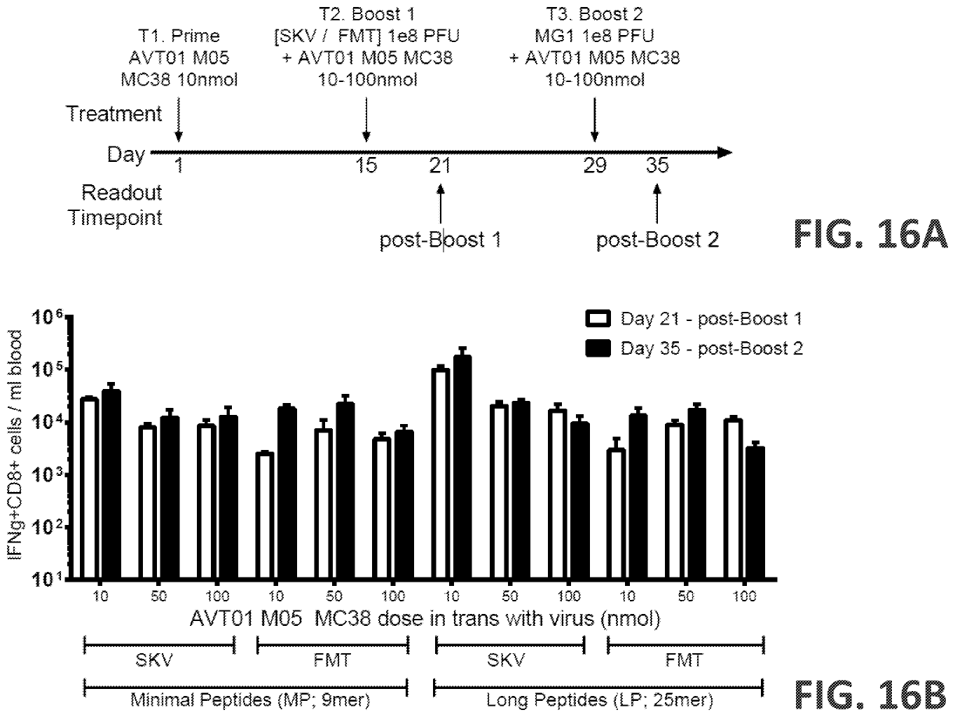

FIG. 16B shows superboost works well at multiple adjuvanted AVT01 M5 (MC38) doses supplied in trans with virus whether using minimum or long peptides. Different dose response where observed for each virus. When using SKV virus during the first boost a lower dose of trans antigen induced the highest number of antigen specific T cells. When using FMT during the first boost, increasing the dose of trans antigen from 10 to 50 nmol was beneficial as it increased antigen specific T cells while increasing the dose further to 100 nmol did not have a benefit. FIG. 16B shows numbers of CD8+ T cells expressing IFNγ in response to ex-vivo stimulation with minimal epitopes (MC3 8 M05) corresponding to antigens used for vaccination in peripheral blood. Mice were primed on day 1 with 10 nmol AVT01 MC38 M05 either short (˜9mer) or long (˜25mer) peptides. Mice were boosted on day 15 intravenously (IV) with 1×10⁸ PFU of either SKV or FMT supplemented with either 10, 50 or 100 nmol AVT01 MC38 M05 either short (˜9mer) or long (˜25mer) peptides. Mice were boosted again on day 29 intravenously (IV) with 1×10⁸ PFU of MG1 supplemented with either 10, 50 or 100 nmol AVT01 MC38 M05 either short (˜9mer) or long (˜25mer) peptides. Blood was sampled on days 21 and 35. See FIG. 16A for the experimental design for the results presented in FIG. 16B.

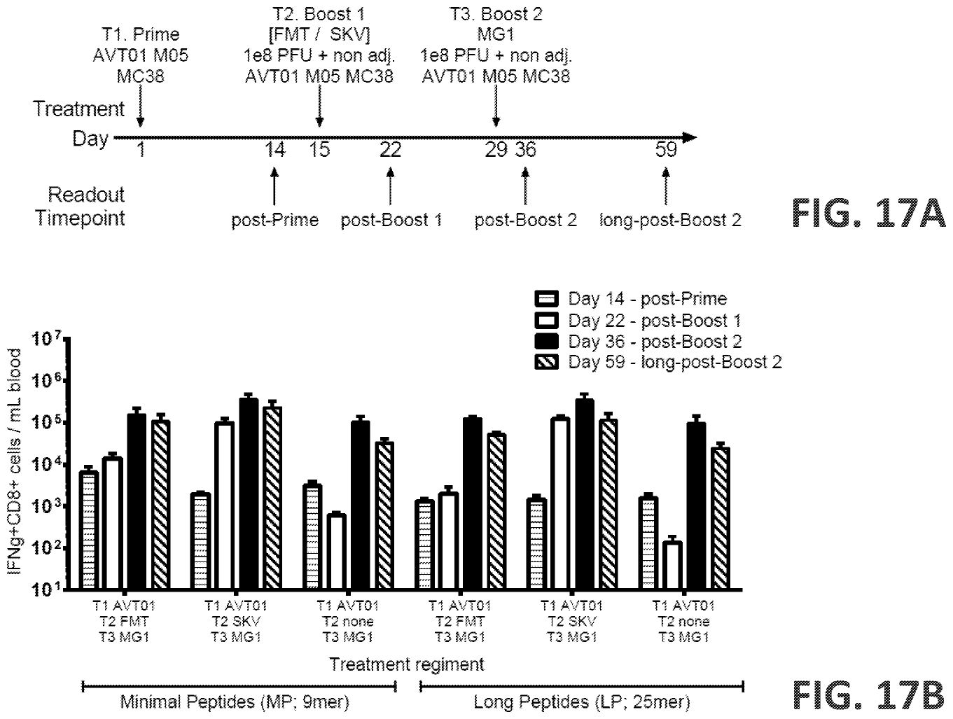

FIG. 17B shows superboost creates a durable response which is maintained 20 days after the last vaccination when non-adjuvanted (no imidazoquinoline-based Toll-like receptor-7 and -8 agonist) AVT01 MC38 M05 either short (˜9mer) or long (˜25mer) peptides ar supplied in trans with the virus. SKV virus increased antigen specific T cells better than FMT during the first boost. FIG. 17B shows numbers of CD8+ T cells expressing IFNγ in response to ex-vivo stimulation with minimal epitopes (MC38 M05) corresponding to antigens used for vaccination in peripheral blood. Mice were primed on day 1 with 10 nmol AVT01 MC38 M05 either short (˜9mer) or long (˜25mer) peptides. Mice were boosted on day 15 intravenously (IV) with 1×10⁸ PFU of either SKV or FMT supplemented with 50 nmol non-adjuvanted AVT01 MC38 M05 either short (˜9mer) or long (˜25mer) peptides. Mice were boosted again on day 29 intravenously (IV) with 1×10⁸ PFU of MG1 supplemented with 50 nmol non-adjuvanted AVT01 MC38 M05 either short (˜9mer) or long (˜25mer) peptides. Blood was sampled on days 14, 22, 36 and 59. See FIG. 17A for the experimental design for the results presented in FIG. 17B.

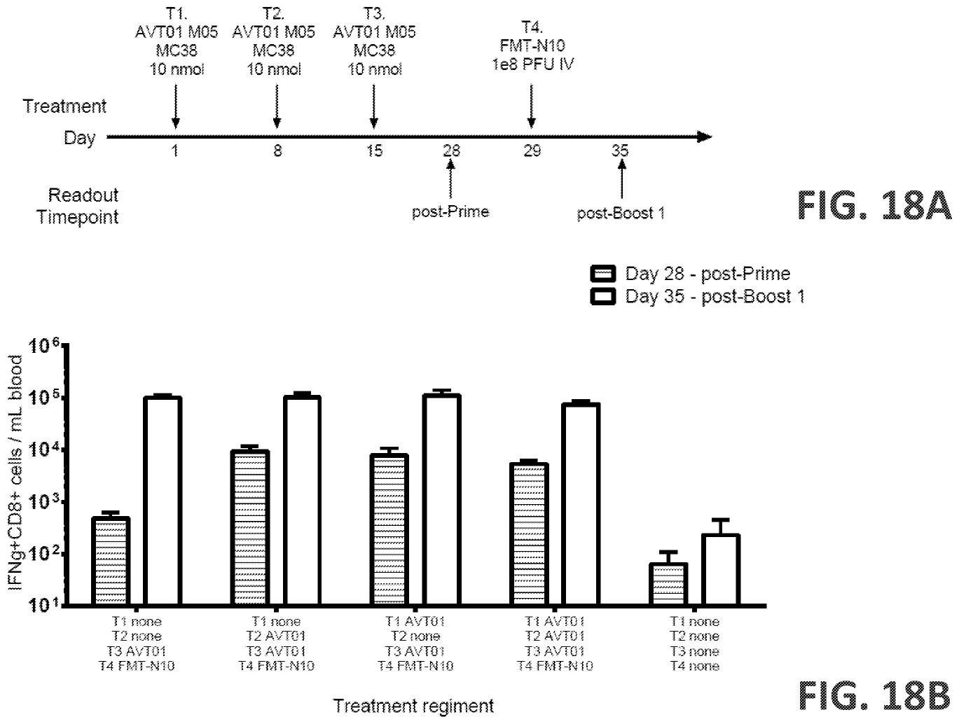

FIG. 18B shows multiple AVT01 MC38 M05 treatments can increase the immune response however, after boost with FMT-N10 all immune responses are similar. Immune response readout of the Adpgk1 neo-antigen as intracellular IFN-γ expression in CD8+ T cells. This figure shows numbers of CD8+ T cells in peripheral blood expressing IFNγ in response to ex-vivo stimulation with Adpgk1 minimal epitope. Mice were either primed or not on days 1, 8 and 15 with AVT01 MC38 M05 long (˜25mer) peptides. Mice were boosted on day 29 intravenously (IV) with 1×10⁸ PFU of FMT-N10. Blood was sampled on days 28 and 35. See FIG. 18A for the experimental design for the results presented in FIG. 18B.

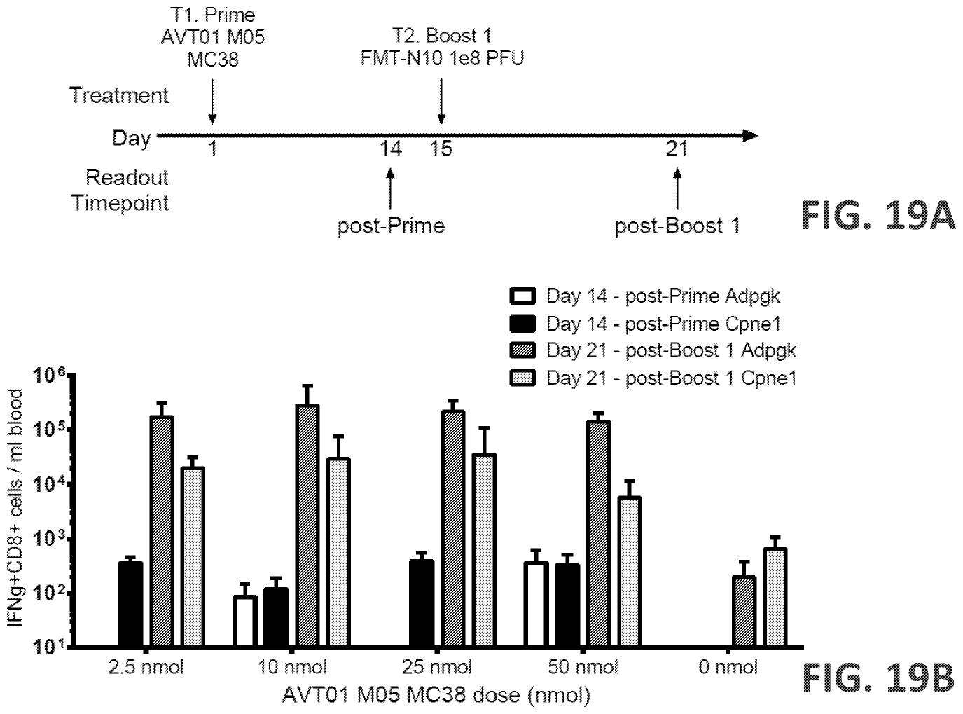

FIG. 19B shows a dose range of AVT01 MC38 M05 prime can be used having a similar effect on immune priming. Immune response readout of the Adpgk1 and Cpne1 neo-antigens as intracellular IFN-γ expression in CD8+ T cells. This figure shows numbers of CD8+ T cells in peripheral blood expressing IFNγ in response to ex-vivo stimulation with either Adpgk1 or Cpne 1 minimal epitope. Mice were primed on day 1 with AVT01 MC38 M05 long (˜25mer) peptides at either 2.5 nmol, 10 nmol, 25 nmol, 50 nmol or 0 nmol (no prime). Mice were boosted on day 15 intravenously (IV) with 1×10⁸ PFU of FMT-N10. Blood was sampled on days 14 and 21. See FIG. 19A for the experimental design for the results presented in FIG. 19B.

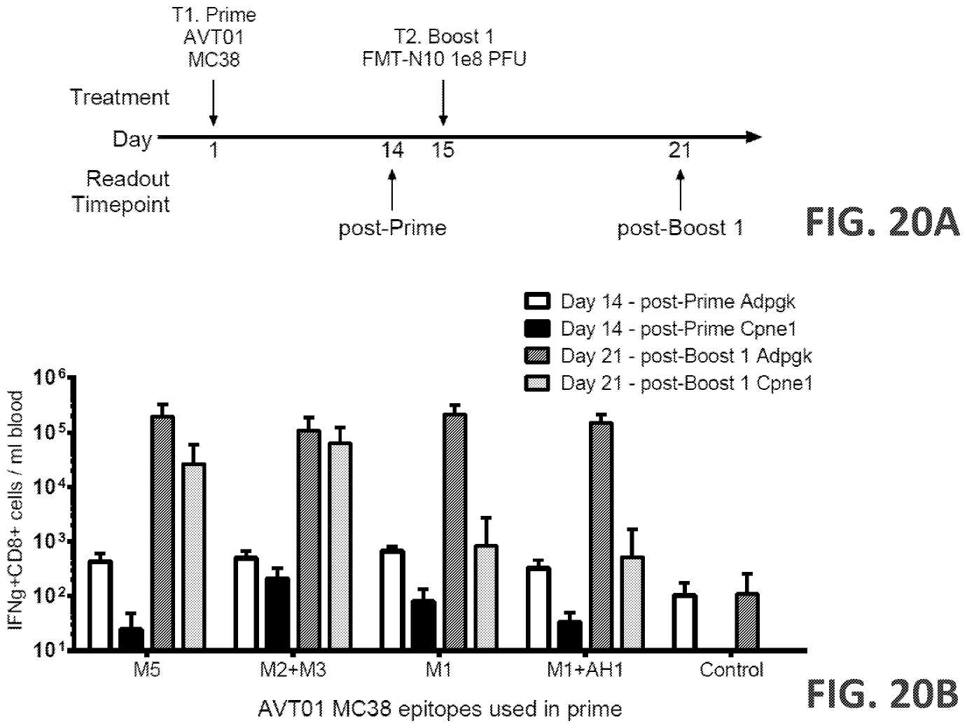

FIG. 20B shows that presence of an irrelevant antigen AH1 during prime does not affect the immune response to the relevant antigen Adpgk. Immune response readout of the Adpgk1 and Cpne1 neo-antigens as intracellular IFN-γ expression in CD8+ Tcells. This figure shows numbers of CD8+ T cells in peripheral blood expressing IFNγ in response to ex-vivo stimulation with either Adpgk1 or Cpne 1 minimal epitope. Mice were primed on day 1 with 10 nmol intramuscularly of either M5 (AVT01 MC38 Adpgk, Irgq, Reps1, Cpne1 and Aatf), M2 (AVT01 MC38 Cpne1 and Aatf) and M3 (AVT01 MC38 Adpgk, Irgq and Reps1), M1 (AVT01 MC38 Adpgk) or M1 (AVT01 MC38 Adpgk) and AH1 (AVT01 AH1) long (˜25mer) peptides. Mice were boosted on day 15 intravenously (IV) with 1×10⁸ PFU of FMT-N10. Blood was sampled on days 14 and 21. See FIG. 20A for the experimental design for the results presented in FIG. 20B.

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. A method of inducing an immune response to at least one neoantigen in a subject, the method comprising:
(a) administering to the subject a dose of a priming composition that is capable of inducing an immune response to the at least one neoantigen, the priming composition comprising a peptide antigen conjugate that comprises (1) an antigenic protein (A) and (2) either a hydrophobic molecule (H) or a particle (P), wherein the antigenic protein (A) is linked to either the hydrophobic molecule (H) or the particle (P) directly or indirectly via an optional N-terminal extension (B1) that is linked to the N-terminus of the antigenic protein (A) or an optional C-terminal extension (B2) that is linked to the C-terminus of the antigenic protein (A), optionally wherein the hydrophobic molecule (H) or Particle (P) is linked to the extension (B1 or B2) indirectly via a Linker (L); and
(b) subsequently administering to the subject a first boost comprising a dose of a first composition, wherein the first composition comprises a first oncolytic virus comprising a genome that comprises a first transgene, wherein the first transgene encodes and expresses a first protein in the subject, and wherein the first protein or a fragment thereof is capable of inducing an immune response to the at least one neoantigen.

2. A method of inducing an immune response to at least one neoantigen in a subject, the method comprising:
(a) administering to the subject a dose of a priming composition that is capable of inducing an immune response to the at least one neoantigen, the priming composition comprising a peptide antigen conjugate that comprises (1) an antigenic protein (A) and (2) either a hydrophobic molecule (H) or a particle (P), wherein the antigenic protein (A) is linked to either the hydrophobic molecule (H) or the particle (P) directly or indirectly via an optional N-terminal extension (B1) that is linked to the N-terminus of the antigenic protein (A) or an optional C-terminal extension (B2) that is linked to the C-terminus of the antigenic protein (A), optionally wherein the hydrophobic molecule (H) or Particle (P) is linked to the extension (B1 or B2) indirectly via a Linker (L); and
(b) administering to the subject a first boost comprising (i) a dose of a first composition comprising a first oncolytic virus and a first peptide composition, or (ii) a dose of a second composition and a dose of a third composition, wherein the second composition comprises the first oncolytic virus, and the third composition comprises the first peptide composition, wherein the first peptide composition is capable of inducing an immune response to the at least one neoantigen, and wherein the second and third compositions are administered concurrently or sequentially to the subject.

3. The method of claim 1, wherein the method further comprises: (c) subsequently administering to the subject a second boost comprising (i) a dose of a second composition, wherein the second composition comprises a second oncolytic virus and a first peptide composition, or (ii) a dose of a third composition and a dose of a fourth composition, wherein the third composition comprises the second oncolytic virus, and the fourth composition comprises the first peptide composition, wherein the first peptide composition is capable of inducing an immune response to the at least one neoantigen, wherein the second oncolytic virus is immunologically distinct from the first oncolytic virus, and wherein the third and fourth compositions are administered concurrently or sequentially to the subject.

4. The method of claim 2, wherein the method further comprises: (c) subsequently administering to the subject a second boost comprising a dose of a fourth composition, wherein the fourth composition comprises a second oncolytic virus that comprises a genome comprising a first transgene, wherein the first transgene encodes and expresses a first protein in the subject, wherein the first protein or a fragment thereof is capable of inducing an immune response to the at least one neoantigen, and wherein the second oncolytic virus is immunologically distinct from the first oncolytic virus.

5. The method of claim 1, wherein the method further comprises: (c) subsequently administering to the subject a second boost comprising a dose of a second composition, wherein the second composition comprises a second oncolytic virus that comprises a genome comprising a second transgene, wherein the second transgene encodes and expresses a second protein in the subject, wherein the second protein or a fragment thereof is capable of inducing an immune response to the at least one neoantigen, and wherein the second oncolytic virus is immunologically distinct from the first oncolytic virus.

6. The method of claim 2, wherein the method further comprises: (c) subsequently administering to the subject a second boost comprising (i) a dose of a fourth composition, wherein the fourth composition comprises a second oncolytic virus and a second peptide composition, or (ii) a dose of a fifth composition and a dose of a sixth composition, wherein the fifth composition comprises the second oncolytic virus, and the sixth composition comprises the second peptide composition, wherein the second peptide composition is capable of inducing an immune response to the at least one neoantigen, wherein the second oncolytic virus is immunologically distinct from the first oncolytic virus, and wherein the fifth and sixth compositions are administered concurrently or sequentially to the subject.

7. A method of inducing an immune response to at least one neoantigen in a subject, the method comprising administering to the subject a first boost comprising a dose of a first composition, wherein the first composition comprises a first oncolytic virus comprising a genome that comprises a first transgene, wherein the first transgene encodes and expresses a first protein in the subject, and wherein the first protein or a fragment thereof is capable of inducing an immune response to the at least one neoantigen,
wherein the subject was previously administered a dose of a priming composition that is capable of inducing an immune response to the least one neoantigen, the priming composition comprising a peptide antigen conjugate that comprises (1) an antigenic protein (A) and (2) either a hydrophobic molecule (H) or a particle (P), wherein the antigenic protein (A) is linked to either the hydrophobic molecule (H) or the particle (P) directly or indirectly via an optional N-terminal extension (B1) that is linked to the N-terminus of the antigenic protein (A) or an optional C-terminal extension (B2) that is linked to the C-terminus of the antigenic protein (A), optionally wherein the hydrophobic molecule (H) or Particle (P) is linked to the extension (B1 or B2) indirectly via a Linker (L).

8. A method of inducing an immune response to at least one neoantigen in a subject, the method comprising administering to the subject a first boost comprising (i) a dose of a first composition comprising a first oncolytic virus and a first peptide composition, or (ii) a dose of a second composition and a dose of a third composition, wherein the second composition comprises the first oncolytic virus, and the third composition comprises the first peptide composition, wherein the first peptide composition is capable of inducing an immune response to the at least one neoantigen, and wherein the second and third compositions are administered concurrently or sequentially to the subject,
wherein the subject was previously administered a dose of a priming composition that is capable of inducing an immune response to the least one neoantigen, the priming composition comprising (1) an antigenic protein (A) and (2) either a hydrophobic molecule (H) or a particle (P), wherein the antigenic protein (A) is linked to either the hydrophobic molecule (H) or the particle (P) directly or indirectly via an optional N-terminal extension (B1) that is linked to the N-terminus of the antigenic protein (A) or an optional C-terminal extension (B2) that is linked to the C-terminus of the antigenic protein (A), optionally wherein the hydrophobic molecule (H) or Particle (P) is linked to the extension (B1 or B2) indirectly via a Linker (L).

9. The method of claim 3, wherein the second boost is administered 7 to 21 days after the first boost.

10. The method of claim 3, wherein the second boost is administered 2 weeks to 3 months after the first boost.

11. The method of claim 5, wherein the second boost is administered 7 to 21 days after the first boost.

12. The method of claim 5, wherein the second boost is administered 2 weeks to 3 months after the first boost.

13. The method of claim 3, 9 or 10, wherein the first oncolytic virus, the second oncolytic virus or both are attenuated.

14. The method of claim 3, 9, 10 or 13, wherein the first oncolytic virus, the second oncolytic viruses, or both are rhabdoviruses.

15. The method of claim 3, 9, 10 or 13, wherein the first oncolytic virus or the second oncolytic virus is a vaccinia virus, an adenovirus, a measles virus, or a vesicular stomatitis virus.

16. The method of claim 15, wherein the vaccinia virus is Copenhagen, Western Reserve, Wyeth, Tian Tan or Lister.

17. The method of claim 3, 9, 10 or 13, wherein the first or second oncolytic virus is a Maraba virus.

18. The method of claim 17, wherein the Maraba virus is MG1.

19. The method of claim 3, 9, 10 or 13, wherein the first or second oncolytic virus is a Farmington virus.

20. The method of claim 3, 9, 10 or 13, wherein the first oncolytic virus is a Maraba virus and the second oncolytic virus is a Farmington virus.

21. The method of claim 3, 9, 10 or 13, wherein the first oncolytic virus is a Farmington virus and the second oncolytic virus is a Maraba virus.

22. The method of claim 3, 9, 10 or 13, wherein the first oncolytic virus is a vaccinia virus and the second oncolytic virus is a Maraba virus.

23. The method of claim 3, 9, 10 or 13, wherein the first oncolytic virus is a Maraba virus and the second oncolytic virus is a vaccinia virus.

24. The method of claim 3, 9, 10 or 13, wherein the first oncolytic virus is a vaccinia virus and the second oncolytic virus is a Farmington virus.

25. The method of claim 3, 9, 10 or 13, wherein the first oncolytic virus is a Farmington virus and the second oncolytic virus is a vaccinia virus.

26. The method of any one of claims 22 to 25, wherein the vaccinia virus is Copenhagen, Western Reserve, Wyeth, Tian Tan or Lister.

27. The method of any one of claims 3, 9, 10 or 13 to 26, wherein the second, third or fourth composition is administered to the subject intravenously, subcutaneously or intramuscularly.

28. The method of claim 5, 11 or 12, wherein the first oncolytic virus, the second oncolytic virus or both are attenuated.

29. The method of claim 5, 11, 12 or 28, wherein the first oncolytic virus, the second oncolytic viruses, or both are rhabdoviruses.

30. The method of claim 5, 11, 12 or 28, wherein the first oncolytic virus or the second oncolytic virus is a vaccinia virus, an adenovirus, a measles virus, or a vesicular stomatitis virus.

31. The method of claim 30, wherein the vaccinia virus is Copenhagen, Western Reserve, Wyeth, Tian Tan or Lister.

32. The method of claim 5, 11, 12 or 28, wherein the first or second oncolytic virus is a Maraba virus.

33. The method of claim 32, wherein the Maraba virus is MG1.

34. The method of claim 5, 11, 12 or 28, wherein the first or second oncolytic virus is a Farmington virus.

35. The method of claim 5, 11, 12 or 28, wherein the first oncolytic virus is a Maraba virus and the second oncolytic virus is a Farmington virus.

36. The method of claim 5, 11, 12 or 28, wherein the first oncolytic virus is a Farmington virus and the second oncolytic virus is a Maraba virus.

37. The method of claim 5, 11, 12 or 28, wherein the first oncolytic virus is a vaccinia virus and the second oncolytic virus is a Maraba virus.

38. The method of claim 5, 11, 12 or 28, wherein the first oncolytic virus is a Maraba virus and the second oncolytic virus is a vaccinia virus.

39. The method of claim 5, 11, 12 or 28, wherein the first oncolytic virus is a vaccinia virus and the second oncolytic virus is a Farmington virus.

40. The method of claim 5, 11, 12 or 28, wherein the first oncolytic virus is a Farmington virus and the second oncolytic virus is a vaccinia virus.

41. The method of any one of claims 37 to 40, wherein the vaccinia virus is Copenhagen, Western Reserve, Wyeth, Tian Tan or Lister.

42. The method of any one of claims 3, 9, 10 or 13 to 27, wherein the second, third or fourth composition is administered to the subject intravenously, subcutaneously or intramuscularly.

43. The method of any one of claims 5, 11, 12, 28 or 28 to 41, wherein the second composition is administered to the subject intravenously, subcutaneously or intramuscularly.

44. The method of any one of claims 1, 3, 5, 7, or 9 to 41, wherein the first composition is administered to the subject intravenously, subcutaneously or intramuscularly.

45. The method of claim 4, wherein the second boost is administered 7 to 21 days after the first boost.

46. The method of claim 4, wherein the second boost is administered 2 weeks to 3 months after the first boost.

47. The method of claim 6, wherein the second boost is administered 7 to 21 days after the first boost.

48. The method of claim 6, wherein the second boost is administered 2 weeks to 3 months after the first boost.

49. The method of claim 4, 45 or 46, wherein the first oncolytic virus, the second oncolytic virus or both are attenuated.

50. The method of claim 4, 45, 46 or 49, wherein the first oncolytic virus, the second oncolytic viruses, or both are rhabdoviruses.

51. The method of claim 4, 45, 46 or 49, wherein the first oncolytic virus or the second oncolytic virus is a vaccinia virus, an adenovirus, a measles virus, or a vesicular stomatitis virus.

52. The method of claim 51, wherein the vaccinia virus is Copenhagen, Western Reserve, Wyeth, Tian Tan or Lister.

53. The method of claim 4, 45, 46 or 49, wherein the first or second oncolytic virus is a Maraba virus.

54. The method of claim 53, wherein the Maraba virus is MG1.

55. The method of claim 4, 45, 46 or 49, wherein the first or second oncolytic virus is a Farmington virus.

56. The method of claim 4, 45, 46 or 49, wherein the first oncolytic virus is a Maraba virus and the second oncolytic virus is a Farmington virus.

57. The method of claim 4, 45, 46 or 49, wherein the first oncolytic virus is a Farmington virus and the second oncolytic virus is a Maraba virus.

58. The method of claim 4, 45, 46 or 49, wherein the first oncolytic virus is a vaccinia virus and the second oncolytic virus is a Maraba virus.

59. The method of claim 4, 45, 46 or 49, wherein the first oncolytic virus is a Maraba virus and the second oncolytic virus is a vaccinia virus.

60. The method of claim 4, 45, 46 or 49, wherein the first oncolytic virus is a vaccinia virus and the second oncolytic virus is a Farmington virus.

61. The method of claim 4, 45, 46 or 49, wherein the first oncolytic virus is a Farmington virus and the second oncolytic virus is a vaccinia virus.

62. The method of any one of claims 58 to 61, wherein the vaccinia virus is Copenhagen, Western Reserve, Wyeth, Tian Tan or Lister.

63. The method of any one of claims 4, 45, 46 or 49 to 62, wherein the fourth composition is administered to the subject intravenously, subcutaneously or intramuscularly.

64. The method of claim 6, 47 or 48, wherein the first oncolytic virus, the second oncolytic virus or both are attenuated.

65. The method of claim 6, 47, 48 or 64, wherein the first oncolytic virus, the second oncolytic viruses, or both are rhabdoviruses.

66. The method of claim 6, 47, 48 or 64, wherein the first oncolytic virus or the second oncolytic virus is a vaccinia virus, an adenovirus, a measles virus, or a vesicular stomatitis virus.

67. The method of claim 66, wherein the vaccinia virus is Copenhagen, Western Reserve, Wyeth, Tian Tan or Lister.

68. The method of claim 6, 47, 48 or 64, wherein the first or second oncolytic virus is a Maraba virus.

69. The method of claim 68, wherein the Maraba virus is MG1.

70. The method of claim 6, 47, 48 or 64, wherein the first or second oncolytic virus is a Farmington virus.

71. The method of claim 6, 47, 48 or 64, wherein the first oncolytic virus is a Maraba virus and the second oncolytic virus is a Farmington virus.

72. The method of claim 6, 47, 48 or 64, wherein the first oncolytic virus is a Farmington virus and the second oncolytic virus is a Maraba virus.

73. The method of claim 6, 47, 48 or 64, wherein the first oncolytic virus is a vaccinia virus and the second oncolytic virus is a Maraba virus.

74. The method of claim 6, 47, 48 or 64, wherein the first oncolytic virus is a Maraba virus and the second oncolytic virus is a vaccinia virus.

75. The method of claim 6, 47, 48 or 64, wherein the first oncolytic virus is a vaccinia virus and the second oncolytic virus is a Farmington virus.

76. The method of claim 6, 47, 48 or 64, wherein the first oncolytic virus is a Farmington virus and the second oncolytic virus is a vaccinia virus.

77. The method of any one of claims 73 to 76, wherein the vaccinia virus is Copenhagen, Western Reserve, Wyeth, Tian Tan or Lister.

78. The method of any one of claims 6, 47, 48 or 64 to 77, wherein the fourth, fifth, or sixth composition is administered to the subject intravenously, subcutaneously or intramuscularly.

79. The method of any one of claims 2, 4, 6, 8 or 45 to 78, wherein the first, second or third composition is administered to the subject intravenously, subcutaneously or intramuscularly.

80. The method of any one of claims 1 to 79, wherein the priming composition is administered to the subject intravenously, subcutaneously or intramuscularly.

81. The method of any one of claims 1 to 80, wherein the dose of the priming composition is administered 7 to 21 days before the first boost.

82. The method of any one of claims 1 to 80, wherein the dose of the priming composition is administered 2 weeks to 3 months before the first boost.

83. The method of any one of claims 1 to 82, wherein the subject has been determined to have pre-existing immunity to the at least one neoantigen.

84. The method of claim 83, wherein the subject is determined to have pre-existing immunity by measuring the number of antigen-specific interferon gamma-positive CD8+ T cells per ml of peripheral blood from the subject.

85. The method of any one of claims 1 to 84, wherein a dose of an oncolytic virus is 10⁷ to 10¹² pfu.

86. The method of any one of claims 1 to 85, wherein the subject is a mammal.

87. The method of any one of claims 1 to 85, wherein the subject is a human.
